(19) United States
(12) Patent Application Publication
(10) Pub. No.: US 2015/0237858 A1
(43) Pub. Date: Aug. 27, 2015

(54) METHOD OF CONTROLLING RYANODINE-MODULATOR INSECTICIDE RESISTANT INSECTS

(71) Applicant: BASF SE, Ludwigshafen (DE)

(72) Inventors: Karsten Koerber, Eppelheim (DE); Jean-Yves Wach, Mannheim (DE); Florian Kaiser, Mannheim (DE); Matthias Pohlman, Freinsheim (DE); Prashant Deshmukh, Mannheim (DE); Deborah L. Culbertson, Fuquay Varina, NC (US); W. David Rogers, Durham, NC (US); Koshi Gunjima, Toyohashi-city (JP); Michael David, Raleigh, NC (US); Franz Josef Braun, Durham, NC (US); Sarah Thompson, Raleigh, NC (US)

(73) Assignee: BASF SE, Ludwigshafen (DE)

(21) Appl. No.: 14/432,039

(22) PCT Filed: Sep. 27, 2013
(86) PCT No.: PCT/EP2013/070161

§ 371 Date: Mar. 27, 2015

Related U.S. Application Data
(60) Provisional application No. 61/708,059, filed on Oct. 1, 2012.
(60) Provisional application No. 61/708,061, filed on Oct. 1, 2012.
(60) Provisional application No. 61/708,066, filed on Oct. 1, 2012.
(60) Provisional application No. 61/708,067, filed on Oct. 1, 2012.
(60) Provisional application No. 61/708,071, filed on Oct. 1, 2012.
(60) Provisional application No. 61/729,350, filed on Nov. 22, 2012.
(60) Provisional application No. 61/764,006, filed on Feb. 13, 2013.
(60) Provisional application No. 61/763,966, filed on Feb. 13, 2013.
(60) Provisional application No. 61/763,970, filed on Feb. 13, 2013.
(60) Provisional application No. 61/763,974, filed on Feb. 13, 2013.
(60) Provisional application No. 61/763,978, filed on Feb. 13, 2013.
(60) Provisional application No. 61/764,083, filed on Feb. 13, 2013.
(60) Provisional application No. 61/767,831, filed on Feb. 22, 2013.

(51) Int. Cl. A01N 43/56; C07D 401/04

(52) U.S. Cl. CPC A01N 43/56; C07D 401/04

(57) ABSTRACT

The present invention relates to a method of controlling insects, especially from the order Lepidoptera, Coleoptera or Diptera, that are resistant to ryanodine-modulator insecticides, especially chlorantraniliprole and cyantraniliprole,
using anthranilamide compounds of formula (I)

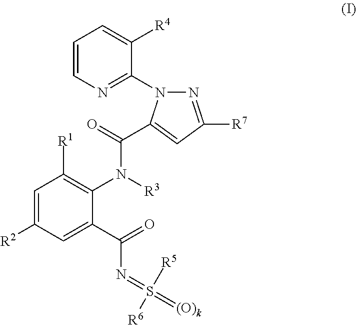

wherein R¹, R², R³, R⁴, R⁵, R⁶, R⁷ and k are as defined in the description;
and their mixtures.

The present invention relates to a method of controlling insects, that are resistant to a ryanodine-modulator insecticide. In particular, the present invention relates to a method of controlling insects from the order Lepidoptera, Coleoptera or Diptera, which are resistant to a ryanodine-modulator insecticide. The present invention also relates to a method of controlling insects from the order Thysanoptera or Homoptera, which are resistant to a ryanodine-modulator insecticide.

The invention relates to a method, in which the compounds of formula (I) itself and their stereoisomers, salts, tautomers or N-oxides, especially their salts, and their mixtures, are used for controlling Lepidoptera or Coleoptera that are resistant to other ryanodine-modulator insecticides and are surprisingly useful in this context.

The present invention is based on the surprising finding that a compound selected from the chemical class of N-thio-anthranilamides can be successfully used to control ryanodine-modulator insecticide resistant populations of arthropods, in particular insects, and more particular insects from the order Lepidoptera, Coleoptera or Diptera, and also insects from the order Thysanoptera or Homoptera.

Thus in the first aspect of the invention there is provided a method of controlling insects which are resistant to a ryanodine-modulator insecticide, which method comprises applying to said ryanodine-modulator insecticide resistant insects at least one pesticidally active anthranilamide compound of formula (I):

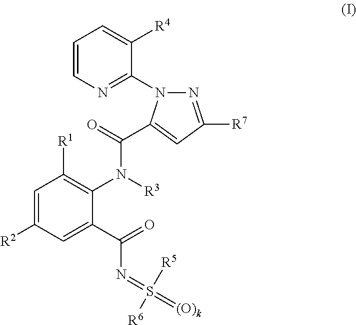

wherein
R¹ is selected from the group consisting of halogen, methyl and halomethyl;
R² is selected from the group consisting of hydrogen, halogen, halomethyl and cyano;
R³ is selected from hydrogen, C₁-C₆ alkyl, C₁-C₆ haloalkyl, C₂-C₆-alkenyl, C₂-C₆-haloalkenyl, C₂-C₆-alkinyl, C₂-C₆-haloalkinyl, C₃-C₈-cycloalkyl, C₃-C₈-halocycloalkyl, C₁-C₄-alkoxy-C₁-C₄-alkyl, C₁-C₄-haloalkoxy-C₁-C₄-alkyl, C(═O)R^{a}, C(═O)OR^{b} and C(═O)NR^{c}R^{d};
R⁴ is hydrogen or halogen;
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
   R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
R⁷ is selected from the group consisting of bromo, chloro, difluoromethyl, trifluoromethyl, nitro, cyano, OCH₃, OCHF₂, OCH₂F, OCH₂CF₃, S(═O)ₙCH₃, and S(═O)ₙCF₃;
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy;
   phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy;
   phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; or
   R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
   two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};
k is 0 or 1;
n is 0, 1 or 2;
or a stereoisomer, salt, tautomer or N-oxide, or a polymorphic crystalline form, a co-crystal or a solvate of a compound or a stereoisomer, salt, tautomer or N-oxide thereof.

Ryanodine-modulator insecticides, and especially the diamide and anthranilamide class, are modern insecticides which are well-known and effective. However, there are some findings that some insects develop resistance even to these modern and effective insecticides (article in press: Troczka, B., et al., Resistance to diamide insecticides in diamondback moth, Plutella xylostella (Lepidoptera: Plutellidae) is associated with a mutation in the membrane-spanning domain of the ryanodine receptor, Insect Biochemistry and Molecular Biology (2012), http://dx.doi.org/10.1016/j.ibmb.2012.09.001). Notably, the authors conclude on a target site resistance of the described pest organism. Surprisingly, it has been found that N-thio anthranilamides of formula I overcome this type of resistance and show excellent efficacy against ryanodine-modulator resistant insects.

Compounds of Formula I

WO 2007/006670, describes N-thio-anthranilamide compounds with a sulfilimine or sulfoximine group and their use as pesticides. PCT/EP2012/065650, PCT/EP2012/065651, and the unpublished applications U.S. 61/578,267, U.S. 61/593,897 and U.S. 61/651,050 describe certain N-Thio-anthranilamide compounds and their use as pesticides.

PCT/EP2012/065648, PCT/EP2012/065649 and EP11189973.8 describe processes for the synthesis of N-Thio-anthranilamide compounds.

However, although the anthranilamide compounds of formula (I) themselves and their combined application with other insecticides are known to have shown activity against certain crop damaging insect pests, the compounds of formula I and some of their selected mixtures with pesticidally active compounds (II) have not yet been described for solving discussed problems as mentioned above.

The compounds of formula I as well as the terms “compounds for methods according to the (present) invention”, “compounds according to the (present) invention” or “compounds of formula (I)” or “compound(s) II”, which all compound(s) are applied in methods and uses according to the present invention comprise the compound(s) as defined herein as well as a known stereoisomer, salt, tautomer or N-oxide thereof (including a polymorphic crystalline form, a co-crystal or a solvate of a compound or a stereoisomer, salt, tautomer or N-oxide thereof).

The term “composition(s) according to the invention” or “composition(s) of the present invention” encompasses composition(s) comprising at least one compound of formula I or mixtures of the compounds of formula I with other pesticidally active compound(s) II for being used and/or applied in methods according to the invention as defined above.

Depending on the substitution pattern, the compounds of the formula (I) may have one or more centers of chirality, in which case they are present as mixtures of enantiomers or diastereomers. The invention provides both the pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond, nitrogen-sulfur double bond or amide group. The term “stereoisomer(s)” encompasses both optical isomers, such as enantiomers or diastereomers, the latter existing due to more than one center of chirality in the molecule, as well as geometrical isomers (cis/trans isomers).

Salts of the compounds of the present invention are preferably agriculturally and veterinarily acceptable salts. They can be formed in a customary method, e.g. by reacting the compound with an acid if the compound of the present invention has a basic functionality or by reacting the compound with a suitable base if the compound of the present invention has an acidic functionality.

In general, suitable “agriculturally useful salts” or “agriculturally acceptable salts” are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The compounds of the formula (I) may be present in the form of their N-oxides. The term “N-oxide” includes any compound of the present invention which has at least one tertiary nitrogen atom that is oxidized to an N-oxide moiety. N-oxides of compounds (I) can in particular be prepared by oxidizing the ring nitrogen atom(s) of the pyridine ring and/or the pyrazole ring with a suitable oxidizing agent, such as peroxo carboxylic acids or other peroxides. The person skilled in the art knows if and in which positions compounds of the formula (I) of the present invention may form N-oxides.

The compounds of the present invention may be amorphous or may exist in one ore more different crystalline states (polymorphs) which may have different macroscopic properties such as stability or show different biological properties such as activities. The present invention includes both amorphous and crystalline compounds of formula (I), their enantiomers or diastereomers, mixtures of different crystalline states of the respective compound of formula (I), its enantiomers or diastereomers, as well as amorphous or crystalline salts thereof.

The term “co-crystal” denotes a complex of the compounds according to the invention or a stereoisomer, salt, tautomer or N-oxide thereof, with one or more other molecules (preferably one molecule type), wherein usually the ratio of the compound according to the invention and the other molecule is a stoichiometric ratio.

The term “solvate” denotes a co-complex of the compounds according to the invention, or a stereoisomer, salt, tautomer or N-oxide thereof, with solvent molecules. The solvent is usually liquid. Examples of solvents are methanol, ethanol, toluol, xylol. A preferred solvent which forms solvates is water, which solvates are referred to as “hydrates”. A solvate or hydrate is usually characterized by the presence of a fixed number of n molecules solvent per m molecules compound according to the invention.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine. A partially or fully halogenated radical is termed below also “halo-radical”. For example, partially or fully halogenated alkyl is also termed haloalkyl.

The term “alkyl” as used herein (and in the alkyl moieties of other groups comprising an alkyl group, e.g. alkoxy, alkylcarbonyl, alkylthio, alkylsulfinyl, alkylsulfonyl and alkoxyalkyl) denotes in each case a straight-chain or branched alkyl group having usually from 1 to 12 or 1 to 10 carbon atoms, frequently from 1 to 6 carbon atoms, preferably 1 to 4 carbon atoms and in particular from 1 to 3 carbon atoms. Examples of C₁-C₄-alkyl are methyl, ethyl, n-propyl, isopropyl, n-butyl, 2-butyl(sec-butyl), isobutyl and tert-butyl. Examples for C₁-C₆-alkyl are, apart those mentioned for C₁-C₄-alkyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Examples for C₁-C₁₀-alkyl are, apart those mentioned for C₁-C₆-alkyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl, 2-propylpentyl, nonyl, decyl, 2-propylheptyl and 3-propylheptyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy, haloalkylthio, haloalkylcarbonyl, haloalkylsulfonyl and haloalkylsulfinyl) denotes in each case a straight-chain or branched alkyl group having usually from 1 to 10 carbon atoms (“C₁-C₁₀-haloalkyl”), frequently from 1 to 6 carbon atoms (“C₁-C₆-haloalkyl”), more frequently 1 to 4 carbon atoms (“C₁-C₁₀-haloalkyl”), wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl. Halomethyl is methyl in which 1, 2 or 3 of the hydrogen atoms are replaced by halogen atoms. Examples are bromomethyl, chloromethyl, dichloromethyl, trichloromethyl, fluoromethyl, difluoromethyl, trifluoromethyl, chlorofluoromethyl, dichlorofluoromethyl, chlorodifluoromethyl and the like. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl, pentafluoroethyl, and the like. Examples for C₁-C₂-haloalkyl are, apart those mentioned for C₁-C₂-fluoroalkyl, chloromethyl, dichloromethyl, trichloromethyl, bromomethyl, chlorofluoromethyl, dichlorofluoromethyl, chlorodifluoromethyl, 1-chloroethyl, 2-chloroethyl, 2,2,-dichloroethyl, 2,2,2-trichloroethyl, 2-chloro-2-fluoroethyl, 2-chloro-2,2-difluoroethyl, 2,2-dichloro-2-fluoroethyl, 1-bromoethyl, and the like. Examples for C₁-C₄-haloalkyl are, apart those mentioned for C₁-C₂-haloalkyl, 1-fluoropropyl, 2-fluoropropyl, 3-fluoropropyl, 3,3-difluoropropyl, 3,3,3-trifluoropropyl, heptafluoropropyl, 1,1,1-trifluoroprop-2-yl, 3-chloropropyl, 4-chlorobutyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms (“C₃-C₁₀-cycloalkyl”), preferably 3 to 8 carbon atoms (“C₃-C₈-cycloalkyl”) or in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.1.1]hexyl, bicyclo[2.2.1]heptyl, bicyclo[3.1.1]heptyl, bicyclo[2.2.1]heptyl, bicyclo[2.2.2]octyl and bicyclo[3.2.1]octyl.

The term “cycloalkylene” (or cycloalkanediyl) as used herein in each case denotes an cycloalkyl radical as defined above, wherein one hydrogen atom at any position of the carbon backbone is replaced by one further binding site, thus forming a bivalent moiety.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, preferably 3 to 8 carbon atoms or in particular 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “cycloalkyl-alkyl” used herein denotes a cycloalkyl group, as defined above, which is bound to the remainder of the molecule via an alkylene group. The term “C₃-C₈-cycloalkyl-C₁-C₄-alkyl” refers to a C₃-C₈-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “alkenyl” as used herein denotes in each case a monounsaturated straight-chain or branched hydrocarbon radical having usually 2 to 10 (“C₂-C₁₀-alkenyl”), preferably 2 to 6 carbon atoms (“C₂-C₆-alkenyl”), in particular 2 to 4 carbon atoms (“C₂-C₄-alkenyl”), and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “alkenylene” (or alkenediyl) as used herein in each case denotes an alkenyl radical as defined above, wherein one hydrogen atom at any position of the carbon backbone is replaced by one further binding site, thus forming a bivalent moiety.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) or 2 to 4 (“C₂-C₄-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “alkynyl” as used herein denotes unsaturated straight-chain or branched hydrocarbon radicals having usually 2 to 10 (“C₂-C₁₀-alkynyl”), frequently 2 to 6 (“C₂-C₆-alkynyl”), preferably 2 to 4 carbon atoms (“C₂-C₄-alkynyl”) and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like.

The term “alkynylene” (or alkynediyl) as used herein in each case denotes an alkynyl radical as defined above, wherein one hydrogen atom at any position of the carbon backbone is replaced by one further binding site, thus forming a bivalent moiety.

The term “haloalkynyl” as used herein, which is also expressed as “alkynyl which may be substituted by halogen”, refers to unsaturated straight-chain or branched hydrocarbon radicals having usually 3 to 10 carbon atoms (“C₂-C₁₀-haloalkynyl”), frequently 2 to 6 (“C₂-C₆-haloalkynyl”), preferably 2 to 4 carbon atoms (“C₂-C₄-haloalkynyl”), and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 10 carbon atoms (“C₁-C₁₀-alkoxy”), frequently from 1 to 6 carbon atoms (“C₁-C₆-alkoxy”), preferably 1 to 4 carbon atoms (“C₁-C₄-alkoxy”), which is bound to the remainder of the molecule via an oxygen atom. C₁-C₂-Alkoxy is methoxy or ethoxy. C₁-C₄-Alkoxy is additionally, for example, n-propoxy, 1-methylethoxy(isopropoxy), butoxy, 1-methylpropoxy(sec-butoxy), 2-methylpropoxy(isobutoxy) or 1,1-dimethylethoxy(tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “haloalkoxy” as used herein denotes in each case a straight-chain or branched alkoxy group, as defined above, having from 1 to 10 carbon atoms (“C₁-C₁₀-haloalkoxy”), frequently from 1 to 6 carbon atoms (“C₁-C₆-haloalkoxy”), preferably 1 to 4 carbon atoms (“C₁-C₄-haloalkoxy”), more preferably 1 to 3 carbon atoms (“C₁-C₃-haloalkoxy”), wherein the hydrogen atoms of this group are partially or totally replaced with halogen atoms, in particular fluorine atoms. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅ C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂-C₂F₅, OCF₂-C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “alkoxyalkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. “C₁-C₆-Alkoxy-C₁-C₆-alkyl” is a C₁-C₆-alkyl group, as defined above, in which one hydrogen atom is replaced by a C₁-C₆-alkoxy group, as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “haloalkoxy-alkyl” as used herein denotes in each case alkyl as defined above, usually comprising 1 to 6 carbon atoms, preferably 1 to 4 carbon atoms, wherein 1 carbon atom carries an haloalkoxy radical as defined above, usually comprising 1 to 10, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl or alkyl-S—)” as used herein denotes in each case a straight-chain or branched saturated alkyl group as defined above, usually comprising 1 to 10 carbon atoms (“C₁-C₁₀-alkylthio”), frequently comprising 1 to 6 carbon atoms (“C₁-C₆-alkylthio”), preferably 1 to 4 carbon atoms (“C₁-C₄-alkylthio”), which is attached via a sulfur atom at any position in the alkyl group. C₁-C₂-Alkylthio is methylthio or ethylthio. C₁-C₄-Alkylthio is additionally, for example, n-propylthio, 1-methylethylthio(isopropylthio), butylthio, 1-methylpropylthio(sec-butylthio), 2-methylpropylthio(isobutylthio) or 1,1-dimethylethylthio(tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “haloalkylthio” as used herein refers to an alkylthio group as defined above wherein the hydrogen atoms are partially or fully substituted by fluorine, chlorine, bromine and/or iodine. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₂-alkylsulfinyl” refers to a C₁-C₂-alkyl group, as defined above, attached via a sulfinyl [S(O)] group. The term “C₁-C₄-alkylsulfinyl” refers to a C₁-C₄-alkyl group, as defined above, attached via a sulfinyl [S(O)] group. The term “C₁-C₆-alkylsulfinyl” refers to a C₁-C₆-alkyl group, as defined above, attached via a sulfinyl [S(O)] group. C₁-C₂-alkylsulfinyl is methylsulfinyl or ethylsulfinyl. C₁-C₄-alkylsulfinyl is additionally, for example, n-propylsulfinyl, 1-methylethylsulfinyl(isopropylsulfinyl), butylsulfinyl, 1-methylpropylsulfinyl(sec-butylsulfinyl), 2-methylpropylsulfinyl(isobutylsulfinyl) or 1,1-dimethylethylsulfinyl(tert-butylsulfinyl). C₁-C₆-alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. The term “C₁-C₂-alkylsulfonyl” refers to a C₁-C₂-alkyl group, as defined above, attached via a sulfonyl [S(O)₂] group. The term “C₁-C₄-alkylsulfonyl” refers to a C₁-C₄-alkyl group, as defined above, attached via a sulfonyl [S(O)₂] group. The term “C₁-C₆-alkylsulfonyl” refers to a C₁-C₆-alkyl group, as defined above, attached via a sulfonyl [S(O)₂] group. C₁-C₂-alkylsulfonyl is methylsulfonyl or ethylsulfonyl. C₁-C₄-alkylsulfonyl is additionally, for example, n-propylsulfonyl, 1-methylethylsulfonyl(isopropylsulfonyl), butylsulfonyl, 1-methylpropylsulfonyl(sec-butylsulfonyl), 2-methylpropylsulfonyl(isobutylsulfonyl) or 1,1-dimethylethylsulfonyl(tert-butylsulfonyl). C₁-C₆-alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “alkylamino” as used herein denotes in each case a group —NHR, wherein R is a straight-chain or branched alkyl group usually having from 1 to 6 carbon atoms (“C₁-C₆-alkylamino”), preferably 1 to 4 carbon atoms(“C₁-C₄-alkylamino”). Examples of C₁-C₆-alkylamino are methylamino, ethylamino, n-propylamino, isopropylamino, n-butylamino, 2-butylamino, iso-butylamino, tert-butylamino, and the like.

The term “dialkylamino” as used herein denotes in each case a group-NRR′, wherein R and R′, independently of each other, are a straight-chain or branched alkyl group each usually having from 1 to 6 carbon atoms (“di-(C₁-C₆-alkyl)-amino”), preferably 1 to 4 carbon atoms (“di-(C₁-C₄-alkyl)-amino”). Examples of a di-(C₁-C₆-alkyl)-amino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The term “cycloalkylamino” as used herein denotes in each case a group —NHR, wherein R is a cycloalkyl group usually having from 3 to 8 carbon atoms (“C₃-C₈-cycloalkylamino”), preferably 3 to 6 carbon atoms (“C₃-C₆-cycloalkylamino”). Examples of C₃-C₈-cycloalkylamino are cyclopropylamino, cyclobutylamino, cyclopentylamino, cyclohexylamino, and the like.

The term “alkylaminosulfonyl” as used herein denotes in each case a straight-chain or branched alkylamino group as defined above, which is bound to the remainder of the molecule via a sulfonyl [S(O)₂] group. Examples of an alkylaminosulfonyl group are methylaminosulfonyl, ethylaminosulfonyl, n-propylaminosulfonyl, isopropylaminosulfonyl, n-butylaminosulfonyl, 2-butylaminosulfonyl, iso-butylaminosulfonyl, tert-butylaminosulfonyl, and the like.

The term “dialkylaminosulfonyl” as used herein denotes in each case a straight-chain or branched alkylamino group as defined above, which is bound to the remainder of the molecule via a sulfonyl [S(O)₂] group. Examples of an dialkylaminosulfonyl group are dimethylaminosulfonyl, diethylaminosulfonyl, dipropylaminosulfonyl, dibutylaminosulfonyl, methyl-ethyl-aminosulfonyl, methyl-propyl-aminosulfonyl, methyl-isopropylaminosulfonyl, methyl-butyl-aminosulfonyl, methyl-isobutyl-aminosulfonyl, ethyl-propyl-aminosulfonyl, ethyl-isopropylaminosulfonyl, ethyl-butyl-aminosulfonyl, ethyl-isobutyl-aminosulfonyl, and the like.

The suffix “-carbonyl” in a group denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “aryl” as used herein refers to a mono-, bi- or tricyclic aromatic hydrocarbon radical such as phenyl or naphthyl, in particular phenyl.

The term “het(ero)aryl” as used herein refers to a mono-, bi- or tricyclic heteroaromatic hydrocarbon radical, preferably to a monocyclic heteroaromatic radical, such as pyridyl, pyrimidyl and the like.

A saturated, partially unsaturated or unsaturated 3- to 8-membered ring system which contains 1 to 4 heteroatoms selected from oxygen, nitrogen, sulfur, is a ring system wherein two oxygen atoms must not be in adjacent positions and wherein at least 1 carbon atom must be in the ring system e.g. thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzthiazole, benzimidazole, benzisoxazole, benzisothiazole, benzopyrazole, benzothiadiazole, benztriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, piperidine, pyrrolidine, oxazoline, tetrahydrofuran, tetrahydropyran, isoxazolidine or thiazolidine, oxirane or oxetane.

A saturated, partially unsaturated or unsaturated 3- to 8-membered ring system which contains 1 to 4 heteroatoms selected from oxygen, nitrogen, sulfur also is e.g.

a saturated, partially unsaturated or unsaturated 5-or 6-membered heterocycle which contains 1 to 4 heteroatoms selected from oxygen, nitrogen and sulfur, such as pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine; or

a saturated, partially unsaturated or unsaturated 5-or 6-membered heterocycle which contains 1 nitrogen atom and 0 to 2 further heteroatoms selected from oxygen, nitrogen and sulfur, preferably from oxygen and nitrogen, such as piperidine, piperazin and morpholine.

Preferably, this ring system is a saturated, partially unsaturated or unsaturated 3- to 6-membered ring system which contains 1 to 4 heteroatoms selected from oxygen, nitrogen, sulfur, wherein two oxygen atoms must not be in adjacent positions and wherein at least 1 carbon atom must be in the ring system.

Most preferably, this ring system is a radical of pyridine, pyrimidine, (1,2,4)-oxadiazole, 1,3,4-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine, oxirane or oxetane.

Preparation of the compounds of formula I can be accomplished according to standard methods of organic chemistry, e.g. by the methods or working examples described in WO 2007/006670, PCT/EP2012/065650 and PCT/EP2012/065651, without being limited to the routes given therein.

The preparation of the compounds of formula I above may lead to them being obtained as isomer mixtures. If desired, these can be resolved by the methods customary for this purpose, such as crystallization or chromatography, also on optically active adsorbate, to give the pure isomers.

Agronomically acceptable salts of the compounds I can be formed in a customary manner, e.g. by reaction with an acid of the anion in question.

Preferences

The remarks made below as to preferred embodiments of the variables (substituents) of the compounds of formulae (I) are valid on their own as well as preferably in combination with each other, as well as in combination with the stereoisomers, tautomers, N-oxides or salts thereof, and, where applicable, as well as concerning the uses and methods according to the invention and the compositions according to the invention.

Preferred compounds according to the invention are compounds of formulae (I) or a stereoisomer, N-oxide or salt thereof, wherein the salt is an agriculturally or veterinarily acceptable salt. The compounds I of formula (I) and their examples include their tautomers, racemic mixtures, individual pure enantiomers and diastereomers and their optically active mixtures.

Preferred are methods and uses of compounds of formula (I), wherein the compound of formula I is a compound of formula IA:

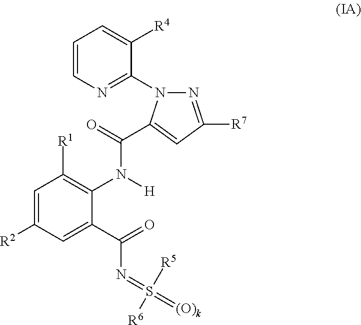

wherein
R⁴ is halogen, and
wherein the variables R¹, R², R⁷, R⁵, R⁶ and k are as defined herein.

Preferred are methods and uses of compounds of formula (I), in which the compound of formula I is a compound of formula IB:

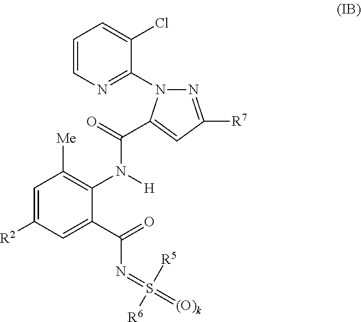

wherein
R² is selected from the group consisting of bromo, chloro, cyano;
R⁷ is selected from the group consisting of bromo, chloro, trifluoromethyl. OCHF₂, and
wherein the variables R², R⁷, R⁵, R⁶ and k are as defined herein.

Preferred are methods and uses of compounds of formula (I), in which the compound of formula I is a compound of formula IC:

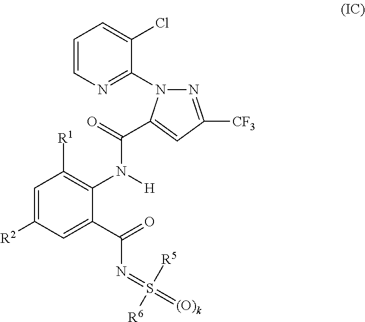

wherein
R¹ is selected from the group consisting of halogen and halomethyl;
R² is selected from the group consisting of bromo, chloro and cyano, and
wherein the variables R⁵, R⁶ and k are as defined herein.

Preferred are methods and uses of compounds of formula (I), in which the compound of formula I is a compound of formula ID:

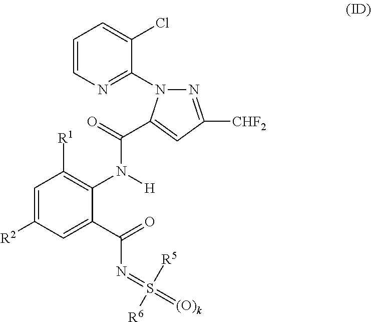

wherein
R¹ is selected from the group consisting of halogen, methyl and halomethyl;
R² is selected from the group consisting of bromo, chloro and cyano, and
wherein the variables R⁵, R⁶ and k are as defined herein.

Preferred are methods and uses of compounds of formula (I), in which R⁵, R⁶ are selected independently of one another from the group consisting of hydrogen, C₁-C₁₀-alkyl, C₃-C₈-cycloalkyl, wherein the aforementioned aliphatic and cycloaliphatic radicals may be substituted with 1 to 10 substituents R^{e}; or

R⁵ and R⁶ together represent a C₂-C₇-alkylene chain forming together with the sulfur atom to which they are attached a 3-, 4-, 5-, 6-, 7- or 8-membered saturated, partially unsaturated or fully unsaturated ring, wherein 1 to 4 of the CH₂ groups in the C₂-C₇-alkylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present.

Preferred are methods and uses of compounds of formula (I), in which R⁵, R⁶ are selected independently of one another from the group consisting of hydrogen, C₁-C₁₀-alkyl, C₃-C₈-cycloalkyl, wherein the aforementioned aliphatic and cycloaliphatic radicals may be substituted with 1 to 10 substituents R^{e}.

Preferred are methods and uses of compounds of formula (I), in which R⁷ is selected from the group consisting of bromo, difluoromethyl, trifluoromethyl, cyano, OCHF₂, OCH₂F and OCH₂CF₃,

Preferred are methods and uses of compounds of formula (I), in which R⁷ is selected from the group consisting of bromo, difluoromethyl, trifluoromethyl and OCHF₂.

Preferred are methods and uses of compounds of formula (I), in which R^{e} is independently selected from the group consisting of halogen, cyano, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy.

Preferred are methods and uses of compounds of formula (I), in which R^{e} is independently selected from the group consisting of halogen, cyano, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated.

Preferred are methods and uses of compounds of formula (I) as described herein, in which in the compound of formula I

R⁵ and R⁶ are selected from methyl, ethyl, isopropyl, n-propyl, n-butyl, isobutyl, tert-butyl, cyclopropyl, cyclopropylmethyl.

Preferred are methods and uses of compounds of formula (I) as described herein, in which in the compound of formula I

R⁵ and R⁶ are identical.

In a particularly preferred embodiment, the methods and uses according to the invention comprise at least one compound of formula (IA)

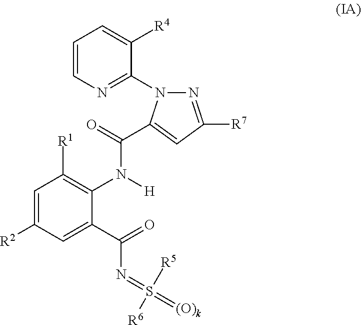

wherein
R⁴ is Cl,
R¹ is selected from the group consisting of Cl, Br, and methyl;
R² is selected from the group consisting of bromo and chloro;
R⁵, R⁶ are selected independently of one another from the group consisting of methyl, ethyl, n-propyl, isopropyl, n-butyl, isobutyl, tert-butyl.
R⁷ is selected from the group consisting of difluoromethyl, trifluoromethyl.

Examples of especially preferred anthranilamide compounds I of the present invention are of formula (IA-1)

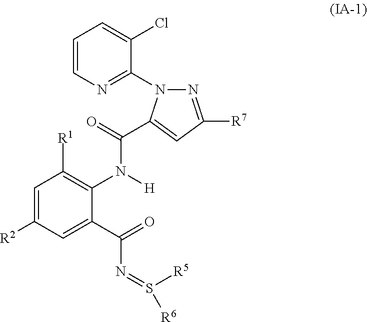

wherein R¹, R², R⁷, R⁵, R⁶ are as defined herein.

Examples of preferred compounds of formula I in the methods and uses according to the invention are compiled in tables 1 to 60 below. Moreover, the meanings mentioned below for the individual variables in the tables are per se, independently of the combination in which they are mentioned, a particularly preferred embodiment of the substituents in question.
Table 1 Compounds of the formula (IA-1) in which R¹ is F, R² is Cl, R⁷ is CF₃ and the combination of R⁵ and R⁶ for a compound corresponds in each case to one row of Table A;
Table 2 Compounds of the formula (IA-1) in which R¹ is Br, R² is Cl, R⁷ is CF₃ and the combination of R⁵ and R⁶ for a compound corresponds in each case to one row of Table A;
Table 3 Compounds of the formula (IA-1) in which R¹ is Cl, R² is Cl, R⁷ is CF₃ and the combination of R⁵ and R⁶ for a compound corresponds in each case to one row of Table A;
Table 4 Compounds of the formula (IA-1) in which R¹ is methyl, R² is Cl, R⁷ is CF₃ and the combination of R⁵ and R⁶ for a compound corresponds in each case to one row of Table A;
Table 5 Compounds of the formula (IA-1) in which R¹ is F, R² is Br, R⁷ is CF₃ and the combination of R⁵ and R⁶ for a compound corresponds in each case to one row of Table A;
Table 6 Compounds of the formula (IA-1) in which R¹ is Br, R² is Br, R⁷ is CF₃ and the combination of R⁵ and R⁶ for a compound corresponds in each case to one row of Table A;
Table 7 Compounds of the formula (IA-1) in which R¹ is Cl, R² is Br, R⁷ is CF₃ and the combination of R⁵ and R⁶ for a compound corresponds in each case to one row of Table A;
Table 8 Compounds of the formula (IA-1) in which R¹ is methyl, R² is Br, R⁷ is CF₃ and the combination of R⁵ and R⁶ for a compound corresponds in each case to one row of Table A;
Table 9 Compounds of the formula (IA-1) in which R¹ is F, R² is cyano, R⁷ is CF₃ and the combination of R⁵ and R⁶ for a compound corresponds in each case to one row of Table A;
Table 10 Compounds of the formula (IA-1) in which R¹ is Br, R² is cyano, R⁷ is CF₃ and the combination of R⁵ and R⁶ for a compound corresponds in each case to one row of Table A;
Table 11 Compounds of the formula (IA-1) in which R¹ is Cl, R² is cyano, R⁷ is CF₃ and the combination of R⁵ and R⁶ for a compound corresponds in each case to one row of Table A;
Table 12 Compounds of the formula (IA-1) in which R¹ is methyl, R² is cyano, R⁷ is CF₃ and the combination of R⁵ and R⁶ for a compound corresponds in each case to one row of Table A;
Table 13 Compounds of the formula (IA-1) in which R¹ is F, R² is Cl, R⁷ is CHF₂ and the combination of R⁵ and R⁶ for a compound corresponds in each case to one row of Table A;
Table 14 Compounds of the formula (IA-1) in which R¹ is Br, R² is Cl, R⁷ is CHF₂ and the combination of R⁵ and R⁶ for a compound corresponds in each case to one row of Table A;
Table 15 Compounds of the formula (IA-1) in which R¹ is Cl, R² is Cl, R⁷ is CHF₂ and the combination of R⁵ and R⁶ for a compound corresponds in each case to one row of Table A;
Table 16 Compounds of the formula (IA-1) in which R¹ is methyl, R² is Cl, R⁷ is CHF₂ and the combination of R⁵ and R⁶ for a compound corresponds in each case to one row of Table A;
Table 17 Compounds of the formula (IA-1) in which R¹ is F, R² is Br, R⁷ is CHF₂ and the combination of R⁵ and R⁶ for a compound corresponds in each case to one row of Table A;
Table 18 Compounds of the formula (IA-1) in which R¹ is Br, R² is Br, R⁷ is CHF₂ and the combination of R⁵ and R⁶ for a compound corresponds in each case to one row of Table A;
Table 19 Compounds of the formula (IA-1) in which R¹ is Cl, R² is Br, R⁷ is CHF₂ and the combination of R⁵ and R⁶ for a compound corresponds in each case to one row of Table A;
Table 20 Compounds of the formula (IA-1) in which R¹ is methyl, R² is Br, R⁷ is CHF₂ and the combination of R⁵ and R⁶ for a compound corresponds in each case to one row of Table A;
Table 21 Compounds of the formula (IA-1) in which R¹ is F, R² is cyano, R⁷ is CHF₂ and the combination of R⁵ and R⁶ for a compound corresponds in each case to one row of Table A;
Table 22 Compounds of the formula (IA-1) in which R¹ is Br, R² is cyano, R⁷ is CHF₂ and the combination of R⁵ and R⁶ for a compound corresponds in each case to one row of Table A;
Table 23 Compounds of the formula (IA-1) in which R¹ is Cl, R² is cyano, R⁷ is CHF₂ and the combination of R⁵ and R⁶ for a compound corresponds in each case to one row of Table A;
Table 24 Compounds of the formula (IA-1) in which R¹ is methyl, R² is cyano, R⁷ is CHF₂ and the combination of R⁵ and R⁶ for a compound corresponds in each case to one row of Table A;
Table 25 Compounds of the formula (IA-1) in which R¹ is F, R² is Cl, R⁷ is Br and the combination of R⁵ and R⁶ for a compound corresponds in each case to one row of Table A;
Table 26 Compounds of the formula (IA-1) in which R¹ is Br, R² is Cl, R⁷ is Br and the combination of R⁵ and R⁶ for a compound corresponds in each case to one row of Table A;
Table 27 Compounds of the formula (IA-1) in which R¹ is Cl, R² is Cl, R⁷ is Br and the combination of R⁵ and R⁶ for a compound corresponds in each case to one row of Table A;
Table 28 Compounds of the formula (IA-1) in which R¹ is methyl, R² is Cl, R⁷ is Br and the combination of R⁵ and R⁶ for a compound corresponds in each case to one row of Table A;
Table 29 Compounds of the formula (IA-1) in which R¹ is F, R² is Br, R⁷ is Br and the combination of R⁵ and R⁶ for a compound corresponds in each case to one row of Table A;
Table 30 Compounds of the formula (IA-1) in which R¹ is Br, R² is Br, R⁷ is Br and the combination of R⁵ and R⁶ for a compound corresponds in each case to one row of Table A;
Table 31 Compounds of the formula (IA-1) in which R¹ is Cl, R² is Br, R⁷ is Br and the combination of R⁵ and R⁶ for a compound corresponds in each case to one row of Table A;
Table 32 Compounds of the formula (IA-1) in which R¹ is methyl, R² is Br, R⁷ is Br and the combination of R⁵ and R⁶ for a compound corresponds in each case to one row of Table A;
Table 33 Compounds of the formula (IA-1) in which R¹ is F, R² is cyano, R⁷ is Br and the combination of R⁵ and R⁶ for a compound corresponds in each case to one row of Table A;
Table 34 Compounds of the formula (IA-1) in which R¹ is Br, R² is cyano, R⁷ is Br and the combination of R⁵ and R⁶ for a compound corresponds in each case to one row of Table A;
Table 35 Compounds of the formula (IA-1) in which R¹ is Cl, R² is cyano, R⁷ is Br and the combination of R⁵ and R⁶ for a compound corresponds in each case to one row of Table A;
Table 36 Compounds of the formula (IA-1) in which R¹ is methyl, R² is cyano, R⁷ is Br and the combination of R⁵ and R⁶ for a compound corresponds in each case to one row of Table A;
Table 37 Compounds of the formula (IA-1) in which R¹ is F, R² is Cl, R⁷ is Cl and the combination of R⁵ and R⁶ for a compound corresponds in each case to one row of Table A;
Table 38 Compounds of the formula (IA-1) in which R¹ is Br, R² is Cl, R⁷ is Cl and the combination of R⁵ and R⁶ for a compound corresponds in each case to one row of Table A;
Table 39 Compounds of the formula (IA-1) in which R¹ is Cl, R² is Cl, R⁷ is Cl and the combination of R⁵ and R⁶ for a compound corresponds in each case to one row of Table A;
Table 40 Compounds of the formula (IA-1) in which R¹ is methyl, R² is Cl, R⁷ is Cl and the combination of R⁵ and R⁶ for a compound corresponds in each case to one row of Table A;
Table 41 Compounds of the formula (IA-1) in which R¹ is F, R² is Br, R⁷ is Cl and the combination of R⁵ and R⁶ for a compound corresponds in each case to one row of Table A;
Table 42 Compounds of the formula (IA-1) in which R¹ is Br, R² is Br, R⁷ is Cl and the combination of R⁵ and R⁶ for a compound corresponds in each case to one row of Table A;
Table 43 Compounds of the formula (IA-1) in which R¹ is Cl, R² is Br, R⁷ is Cl and the combination of R⁵ and R⁶ for a compound corresponds in each case to one row of Table A;
Table 44 Compounds of the formula (IA-1) in which R¹ is methyl, R² is Br, R⁷ is Cl and the combination of R⁵ and R⁶ for a compound corresponds in each case to one row of Table A;
Table 45 Compounds of the formula (IA-1) in which R¹ is F, R² is cyano, R⁷ is Cl and the combination of R⁵ and R⁶ for a compound corresponds in each case to one row of Table A;
Table 46 Compounds of the formula (IA-1) in which R¹ is Br, R² is cyano, R⁷ is Cl and the combination of R⁵ and R⁶ for a compound corresponds in each case to one row of Table A;
Table 47 Compounds of the formula (IA-1) in which R¹ is Cl, R² is cyano, R⁷ is Cl and the combination of R⁵ and R⁶ for a compound corresponds in each case to one row of Table A;
Table 48 Compounds of the formula (IA-1) in which R¹ is methyl, R² is cyano, R⁷ is Cl and the combination of R⁵ and R⁶ for a compound corresponds in each case to one row of Table A;
Table 49 Compounds of the formula (IA-1) in which R¹ is F, R² is Cl, R⁷ is OCHF₂ and the combination of R⁵ and R⁶ for a compound corresponds in each case to one row of Table A;
Table 50 Compounds of the formula (IA-1) in which R¹ is Br, R² is Cl, R⁷ is OCHF₂ and the combination of R⁵ and R⁶ for a compound corresponds in each case to one row of Table A;
Table 51 Compounds of the formula (IA-1) in which R¹ is Cl, R² is Cl, R⁷ is OCHF₂ and the combination of R⁵ and R⁶ for a compound corresponds in each case to one row of Table A;
Table 52 Compounds of the formula (IA-1) in which R¹ is methyl, R² is Cl, R⁷ is OCHF₂ and the combination of R⁵ and R⁶ for a compound corresponds in each case to one row of Table A;
Table 53 Compounds of the formula (IA-1) in which R¹ is F, R² is Br, R⁷ is OCHF₂ and the combination of R⁵ and R⁶ for a compound corresponds in each case to one row of Table A;
Table 54 Compounds of the formula (IA-1) in which R¹ is Br, R² is Br, R⁷ is OCHF₂ and the combination of R⁵ and R⁶ for a compound corresponds in each case to one row of Table A;
Table 55 Compounds of the formula (IA-1) in which R¹ is Cl, R² is Br, R⁷ is OCHF₂ and the combination of R⁵ and R⁶ for a compound corresponds in each case to one row of Table A;
Table 56 Compounds of the formula (IA-1) in which R¹ is methyl, R² is Br, R⁷ is OCHF₂ and the combination of R⁵ and R⁶ for a compound corresponds in each case to one row of Table A;
Table 57 Compounds of the formula (IA-1) in which R¹ is F, R² is cyano, R⁷ is OCHF₂ and the combination of R⁵ and R⁶ for a compound corresponds in each case to one row of Table A;
Table 58 Compounds of the formula (IA-1) in which R¹ is Br, R² is cyano, R⁷ is OCHF₂ and the combination of R⁵ and R⁶ for a compound corresponds in each case to one row of Table A;
Table 59 Compounds of the formula (IA-1) in which R¹ is Cl, R² is cyano, R⁷ is OCHF₂ and the combination of R⁵ and R⁶ for a compound corresponds in each case to one row of Table A;
Table 60 Compounds of the formula (IA-1) in which R¹ is methyl, R² is cyano, R⁷ is OCHF₂ and the combination of R⁵ and R⁶ for a compound corresponds in each case to one row of Table A.

TABLE A

| | R⁵ | R⁶ |
|---|---|---|
| A-1 | CH₃ | CH₃ |
| A-2 | C₂H₅ | CH₃ |
| A-3 | CH═CH₂ | CH₃ |
| A-4 | CH₂CH₂CH₃ | CH₃ |
| A-5 | CH(CH₃)₂ | CH₃ |
| A-6 | CH₂CH₂CH₂CH₃ | CH₃ |
| A-7 | C(CH₃)₃ | CH₃ |
| A-8 | CH₂CH(CH₃)₂ | CH₃ |
| A-9 | CH(CH₃)CH₂CH₃ | CH₃ |
| A-10 | CH₂CH═CH₂ | CH₃ |
| A-11 | CH₂C≡CH | CH₃ |
| A-12 | CH(CH₃)CH═CH₂ | CH₃ |
| A-13 | CHF₂ | CH₃ |
| A-14 | CH₂Cl | CH₃ |
| A-15 | CH₂CH₂CN | CH₃ |
| A-16 | CH₂CH₂Cl | CH₃ |
| A-17 | c-C₃H₅ | CH₃ |
| A-18 | c-C₄H₇ | CH₃ |
| A-19 | c-C₅H₉ | CH₃ |
| A-20 | c-C₆H₁₁ | CH₃ |
| A-21 | CH₂—c-C₃H₅ | CH₃ |
| A-22 | CH(CH₃)—c-C₃H₅ | CH₃ |
| A-23 | CH₂—c-C₅H₉ | CH₃ |
| A-24 | CH₂—c-C₆H₁₁ | CH₃ |
| A-25 | C₆H₅ | CH₃ |
| A-26 | CH₃ | C₂H₅ |
| A-27 | C₂H₅ | C₂H₅ |
| A-28 | CH═CH₂ | C₂H₅ |
| A-29 | CH₂CH₂CH₃ | C₂H₅ |
| A-30 | CH(CH₃)₂ | C₂H₅ |
| A-31 | CH₂CH₂CH₂CH₃ | C₂H₅ |
| A-32 | C(CH₃)₃ | C₂H₅ |
| A-33 | CH₂CH(CH₃)₂ | C₂H₅ |
| A-34 | CH(CH₃)CH₂CH₃ | C₂H₅ |
| A-35 | CH₂CH═CH₂ | C₂H₅ |
| A-36 | CH₂C≡CH | C₂H₅ |
| A-37 | CH(CH₃)CH═CH₂ | C₂H₅ |
| A-38 | CHF₂ | C₂H₅ |
| A-39 | CH₂Cl | C₂H₅ |
| A-40 | CH₂CH₂CN | C₂H₅ |
| A-41 | CH₂CH₂Cl | C₂H₅ |
| A-42 | c-C₃H₅ | C₂H₅ |
| A-43 | c-C₄H₇ | C₂H₅ |
| A-44 | c-C₅H₉ | C₂H₅ |
| A-45 | c-C₆H₁₁ | C₂H₅ |
| A-46 | CH₂—c-C₃H₅ | C₂H₅ |
| A-47 | CH(CH₃)—c-C₃H₅ | C₂H₅ |
| A-48 | CH₂—c-C₅H₉ | C₂H₅ |
| A-49 | CH₂—c-C₆H₁₁ | C₂H₅ |
| A-50 | C₆H₅ | C₂H₅ |
| A-51 | CH₃ | CH═CH₂ |
| A-52 | C₂H₅ | CH═CH₂ |
| A-53 | CH═CH₂ | CH═CH₂ |
| A-54 | CH₂CH₂CH₃ | CH═CH₂ |
| A-55 | CH(CH₃)₂ | CH═CH₂ |
| A-56 | CH₂CH₂CH₂CH₃ | CH═CH₂ |
| A-57 | C(CH₃)₃ | CH═CH₂ |
| A-58 | CH₂CH(CH₃)₂ | CH═CH₂ |
| A-59 | CH(CH₃)CH₂CH₃ | CH═CH₂ |
| A-60 | CH₂CH═CH₂ | CH═CH₂ |
| A-61 | CH₂C≡CH | CH═CH₂ |
| A-62 | CH(CH₃)CH═CH₂ | CH═CH₂ |
| A-63 | CHF₂ | CH═CH₂ |
| A-64 | CH₂Cl | CH═CH₂ |
| A-65 | CH₂CH₂CN | CH═CH₂ |
| A-66 | CH₂CH₂Cl | CH═CH₂ |
| A-67 | c-C₃H₅ | CH═CH₂ |
| A-68 | c-C₄H₇ | CH═CH₂ |
| A-69 | c-C₅H₉ | CH═CH₂ |
| A-70 | c-C₆H₁₁ | CH═CH₂ |
| A-71 | CH₂—c-C₃H₅ | CH═CH₂ |
| A-72 | CH(CH₃)—c-C₃H₅ | CH═CH₂ |
| A-73 | CH₂—c-C₅H₉ | CH═CH₂ |
| A-74 | CH₂—c-C₆H₁₁ | CH═CH₂ |
| A-75 | C₆H₅ | CH═CH₂ |
| A-76 | CH₃ | CH₂CH₂CH₃ |
| A-77 | C₂H₅ | CH₂CH₂CH₃ |
| A-78 | CH═CH₂ | CH₂CH₂CH₃ |
| A-79 | CH₂CH₂CH₃ | CH₂CH₂CH₃ |
| A-80 | CH(CH₃)₂ | CH₂CH₂CH₃ |
| A-81 | CH₂CH₂CH₂CH₃ | CH₂CH₂CH₃ |
| A-82 | C(CH₃)₃ | CH₂CH₂CH₃ |
| A-83 | CH₂CH(CH₃)₂ | CH₂CH₂CH₃ |
| A-84 | CH(CH₃)CH₂CH₃ | CH₂CH₂CH₃ |
| A-85 | CH₂CH═CH₂ | CH₂CH₂CH₃ |
| A-86 | CH₂C≡CH | CH₂CH₂CH₃ |
| A-87 | CH(CH₃)CH═CH₂ | CH₂CH₂CH₃ |
| A-88 | CHF₂ | CH₂CH₂CH₃ |
| A-89 | CH₂Cl | CH₂CH₂CH₃ |
| A-90 | CH₂CH₂CN | CH₂CH₂CH₃ |
| A-91 | CH₂CH₂Cl | CH₂CH₂CH₃ |
| A-92 | c-C₃H₅ | CH₂CH₂CH₃ |
| A-93 | c-C₄H₇ | CH₂CH₂CH₃ |
| A-94 | c-C₅H₉ | CH₂CH₂CH₃ |
| A-95 | c-C₆H₁₁ | CH₂CH₂CH₃ |
| A-96 | CH₂—c-C₃H₅ | CH₂CH₂CH₃ |
| A-97 | CH(CH₃)—c-C₃H₅ | CH₂CH₂CH₃ |
| A-98 | CH₂—c-C₅H₉ | CH₂CH₂CH₃ |
| A-99 | CH₂—c-C₆H₁₁ | CH₂CH₂CH₃ |
| A-100 | C₆H₅ | CH₂CH₂CH₃ |
| A-101 | CH₃ | CH(CH₃)₂ |
| A-102 | C₂H₅ | CH(CH₃)₂ |
| A-103 | CH═CH₂ | CH(CH₃)₂ |
| A-104 | CH₂CH₂CH₃ | CH(CH₃)₂ |
| A-105 | CH(CH₃)₂ | CH(CH₃)₂ |
| A-106 | CH₂CH₂CH₂CH₃ | CH(CH₃)₂ |
| A-107 | C(CH₃)₃ | CH(CH₃)₂ |
| A-108 | CH₂CH(CH₃)₂ | CH(CH₃)₂ |
| A-109 | CH(CH₃)CH₂CH₃ | CH(CH₃)₂ |
| A-110 | CH₂CH═CH₂ | CH(CH₃)₂ |
| A-111 | CH₂C≡CH | CH(CH₃)₂ |
| A-112 | CH(CH₃)CH═CH₂ | CH(CH₃)₂ |
| A-113 | CHF₂ | CH(CH₃)₂ |
| A-114 | CH₂Cl | CH(CH₃)₂ |
| A-115 | CH₂CH₂CN | CH(CH₃)₂ |
| A-116 | CH₂CH₂Cl | CH(CH₃)₂ |
| A-117 | c-C₃H₅ | CH(CH₃)₂ |
| A-118 | c-C₄H₇ | CH(CH₃)₂ |
| A-119 | c-C₅H₉ | CH(CH₃)₂ |
| A-120 | c-C₆H₁₁ | CH(CH₃)₂ |
| A-121 | CH₂—c-C₃H₅ | CH(CH₃)₂ |
| A-122 | CH(CH₃)—c-C₃H₅ | CH(CH₃)₂ |
| A-123 | CH₂—c-C₅H₉ | CH(CH₃)₂ |
| A-124 | CH₂—c-C₆H₁₁ | CH(CH₃)₂ |
| A-125 | C₆H₅ | CH(CH₃)₂ |
| A-126 | CH₃ | CH₂CH₂CH₂CH₃ |
| A-127 | C₂H₅ | CH₂CH₂CH₂CH₃ |
| A-128 | CH═CH₂ | CH₂CH₂CH₂CH₃ |
| A-129 | CH₂CH₂CH₃ | CH₂CH₂CH₂CH₃ |
| A-130 | CH(CH₃)₂ | CH₂CH₂CH₂CH₃ |
| A-131 | CH₂CH₂CH₂CH₃ | CH₂CH₂CH₂CH₃ |
| A-132 | C(CH₃)₃ | CH₂CH₂CH₂CH₃ |
| A-133 | CH₂CH(CH₃)₂ | CH₂CH₂CH₂CH₃ |
| A-134 | CH(CH₃)CH₂CH₃ | CH₂CH₂CH₂CH₃ |
| A-135 | CH₂CH═CH₂ | CH₂CH₂CH₂CH₃ |
| A-136 | CH₂C≡CH | CH₂CH₂CH₂CH₃ |
| A-137 | CH(CH₃)CH═CH₂ | CH₂CH₂CH₂CH₃ |
| A-138 | CHF₂ | CH₂CH₂CH₂CH₃ |
| A-139 | CH₂Cl | CH₂CH₂CH₂CH₃ |
| A-140 | CH₂CH₂CN | CH₂CH₂CH₂CH₃ |
| A-141 | CH₂CH₂Cl | CH₂CH₂CH₂CH₃ |
| A-142 | c-C₃H₅ | CH₂CH₂CH₂CH₃ |
| A-143 | c-C₄H₇ | CH₂CH₂CH₂CH₃ |
| A-144 | c-C₅H₉ | CH₂CH₂CH₂CH₃ |
| A-145 | c-C₆H₁₁ | CH₂CH₂CH₂CH₃ |
| A-146 | CH₂—c-C₃H₅ | CH₂CH₂CH₂CH₃ |
| A-147 | CH(CH₃)—c-C₃H₅ | CH₂CH₂CH₂CH₃ |
| A-148 | CH₂—c-C₅H₉ | CH₂CH₂CH₂CH₃ |
| A-149 | CH₂—c-C₆H₁₁ | CH₂CH₂CH₂CH₃ |
| A-150 | C₆H₅ | CH₂CH₂CH₂CH₃ |
| A-151 | CH₃ | C(CH₃)₃ |
| A-152 | C₂H₅ | C(CH₃)₃ |
| A-153 | CH═CH₂ | C(CH₃)₃ |
| A-154 | CH₂CH₂CH₃ | C(CH₃)₃ |
| A-155 | CH(CH₃)₂ | C(CH₃)₃ |
| A-156 | CH₂CH₂CH₂CH₃ | C(CH₃)₃ |
| A-157 | C(CH₃)₃ | C(CH₃)₃ |
| A-158 | CH₂CH(CH₃)₂ | C(CH₃)₃ |
| A-159 | CH(CH₃)CH₂CH₃ | C(CH₃)₃ |
| A-160 | CH₂CH═CH₂ | C(CH₃)₃ |
| A-161 | CH₂C≡CH | C(CH₃)₃ |
| A-162 | CH(CH₃)CH═CH₂ | C(CH₃)₃ |
| A-163 | CHF₂ | C(CH₃)₃ |
| A-164 | CH₂Cl | C(CH₃)₃ |
| A-165 | CH₂CH₂CN | C(CH₃)₃ |
| A-166 | CH₂CH₂Cl | C(CH₃)₃ |
| A-167 | c-C₃H₅ | C(CH₃)₃ |
| A-168 | c-C₄H₇ | C(CH₃)₃ |
| A-169 | c-C₅H₉ | C(CH₃)₃ |
| A-170 | c-C₆H₁₁ | C(CH₃)₃ |
| A-171 | CH₂—c-C₃H₅ | C(CH₃)₃ |
| A-172 | CH(CH₃)—c-C₃H₅ | C(CH₃)₃ |
| A-173 | CH₂—c-C₅H₉ | C(CH₃)₃ |
| A-174 | CH₂—c-C₆H₁₁ | C(CH₃)₃ |
| A-175 | C₆H₅ | C(CH₃)₃ |
| A-176 | CH₃ | CH₂CH(CH₃)₂ |
| A-177 | C₂H₅ | CH₂CH(CH₃)₂ |
| A-178 | CH═CH₂ | CH₂CH(CH₃)₂ |
| A-179 | CH₂CH₂CH₃ | CH₂CH(CH₃)₂ |
| A-180 | CH(CH₃)₂ | CH₂CH(CH₃)₂ |
| A-181 | CH₂CH₂CH₂CH₃ | CH₂CH(CH₃)₂ |
| A-182 | C(CH₃)₃ | CH₂CH(CH₃)₂ |
| A-183 | CH₂CH(CH₃)₂ | CH₂CH(CH₃)₂ |
| A-184 | CH(CH₃)CH₂CH₃ | CH₂CH(CH₃)₂ |
| A-185 | CH₂CH═CH₂ | CH₂CH(CH₃)₂ |
| A-186 | CH₂C≡CH | CH₂CH(CH₃)₂ |
| A-187 | CH(CH₃)CH═CH₂ | CH₂CH(CH₃)₂ |
| A-188 | CHF₂ | CH₂CH(CH₃)₂ |
| A-189 | CH₂Cl | CH₂CH(CH₃)₂ |
| A-190 | CH₂CH₂CN | CH₂CH(CH₃)₂ |
| A-191 | CH₂CH₂Cl | CH₂CH(CH₃)₂ |
| A-192 | c-C₃H₅ | CH₂CH(CH₃)₂ |
| A-193 | c-C₄H₇ | CH₂CH(CH₃)₂ |
| A-194 | c-C₅H₉ | CH₂CH(CH₃)₂ |
| A-195 | c-C₆H₁₁ | CH₂CH(CH₃)₂ |
| A-196 | CH₂—c-C₃H₅ | CH₂CH(CH₃)₂ |
| A-197 | CH(CH₃)—c-C₃H₅ | CH₂CH(CH₃)₂ |
| A-198 | CH₂—c-C₅H₉ | CH₂CH(CH₃)₂ |
| A-199 | CH₂—c-C₆H₁₁ | CH₂CH(CH₃)₂ |
| A-200 | C₆H₅ | CH₂CH(CH₃)₂ |
| A-201 | CH₃ | CH(CH₃)CH₂CH₃ |
| A-202 | C₂H₅ | CH(CH₃)CH₂CH₃ |
| A-203 | CH═CH₂ | CH(CH₃)CH₂CH₃ |
| A-204 | CH₂CH₂CH₃ | CH(CH₃)CH₂CH₃ |
| A-205 | CH(CH₃)₂ | CH(CH₃)CH₂CH₃ |
| A-206 | CH₂CH₂CH₂CH₃ | CH(CH₃)CH₂CH₃ |
| A-207 | C(CH₃)₃ | CH(CH₃)CH₂CH₃ |
| A-208 | CH₂CH(CH₃)₂ | CH(CH₃)CH₂CH₃ |
| A-209 | CH(CH₃)CH₂CH₃ | CH(CH₃)CH₂CH₃ |
| A-210 | CH₂CH═CH₂ | CH(CH₃)CH₂CH₃ |
| A-211 | CH₂C≡CH | CH(CH₃)CH₂CH₃ |
| A-212 | CH(CH₃)CH═CH₂ | CH(CH₃)CH₂CH₃ |
| A-213 | CHF₂ | CH(CH₃)CH₂CH₃ |
| A-214 | CH₂Cl | CH(CH₃)CH₂CH₃ |
| A-215 | CH₂CH₂CN | CH(CH₃)CH₂CH₃ |
| A-216 | CH₂CH₂Cl | CH(CH₃)CH₂CH₃ |
| A-217 | c-C₃H₅ | CH(CH₃)CH₂CH₃ |
| A-218 | c-C₄H₇ | CH(CH₃)CH₂CH₃ |
| A-219 | c-C₅H₉ | CH(CH₃)CH₂CH₃ |
| A-220 | c-C₆H₁₁ | CH(CH₃)CH₂CH₃ |
| A-221 | CH₂—c-C₃H₅ | CH(CH₃)CH₂CH₃ |
| A-222 | CH(CH₃)—c-C₃H₅ | CH(CH₃)CH₂CH₃ |
| A-223 | CH₂—c-C₅H₉ | CH(CH₃)CH₂CH₃ |
| A-224 | CH₂—c-C₆H₁₁ | CH(CH₃)CH₂CH₃ |
| A-225 | C₆H₅ | CH(CH₃)CH₂CH₃ |
| A-226 | CH₃ | CH₂CH═CH₂ |
| A-227 | C₂H₅ | CH₂CH═CH₂ |
| A-228 | CH═CH₂ | CH₂CH═CH₂ |
| A-229 | CH₂CH₂CH₃ | CH₂CH═CH₂ |
| A-230 | CH(CH₃)₂ | CH₂CH═CH₂ |
| A-231 | CH₂CH₂CH₂CH₃ | CH₂CH═CH₂ |
| A-232 | C(CH₃)₃ | CH₂CH═CH₂ |
| A-233 | CH₂CH(CH₃)₂ | CH₂CH═CH₂ |
| A-234 | CH(CH₃)CH₂CH₃ | CH₂CH═CH₂ |
| A-235 | CH₂CH═CH₂ | CH₂CH═CH₂ |
| A-236 | CH₂C≡CH | CH₂CH═CH₂ |
| A-237 | CH(CH₃)CH═CH₂ | CH₂CH═CH₂ |
| A-238 | CHF₂ | CH₂CH═CH₂ |
| A-239 | CH₂Cl | CH₂CH═CH₂ |
| A-240 | CH₂CH₂CN | CH₂CH═CH₂ |
| A-241 | CH₂CH₂Cl | CH₂CH═CH₂ |
| A-242 | c-C₃H₅ | CH₂CH═CH₂ |
| A-243 | c-C₄H₇ | CH₂CH═CH₂ |
| A-244 | c-C₅H₉ | CH₂CH═CH₂ |
| A-245 | c-C₆H₁₁ | CH₂CH═CH₂ |
| A-246 | CH₂—c-C₃H₅ | CH₂CH═CH₂ |
| A-247 | CH(CH₃)—c-C₃H₅ | CH₂CH═CH₂ |
| A-248 | CH₂—c-C₅H₉ | CH₂CH═CH₂ |
| A-249 | CH₂—c-C₆H₁₁ | CH₂CH═CH₂ |
| A-250 | C₆H₅ | CH₂CH═CH₂ |
| A-251 | CH₃ | CH₂C≡CH |
| A-252 | C₂H₅ | CH₂C≡CH |
| A-253 | CH═CH₂ | CH₂C≡CH |
| A-254 | CH₂CH₂CH₃ | CH₂C≡CH |
| A-255 | CH(CH₃)₂ | CH₂C≡CH |
| A-256 | CH₂CH₂CH₂CH₃ | CH₂C≡CH |
| A-257 | C(CH₃)₃ | CH₂C≡CH |
| A-258 | CH₂CH(CH₃)₂ | CH₂C≡CH |
| A-259 | CH(CH₃)CH₂CH₃ | CH₂C≡CH |
| A-260 | CH₂CH═CH₂ | CH₂C≡CH |
| A-261 | CH2C≡CH | CH₂C≡CH |
| A-262 | CH(CH₃)CH═CH₂ | CH₂C≡CH |
| A-263 | CHF₂ | CH₂C≡CH |
| A-264 | CH₂Cl | CH₂C≡CH |
| A-265 | CH₂CH₂CN | CH₂C≡CH |
| A-266 | CH₂CH₂Cl | CH₂C≡CH |
| A-267 | c-C₃H₅ | CH₂C≡CH |
| A-268 | c-C₄H₇ | CH₂C≡CH |
| A-269 | c-C₅H₉ | CH₂C≡CH |
| A-270 | c-C₆H₁₁ | CH₂C≡CH |
| A-271 | CH₂—c-C₃H₅ | CH₂C≡CH |
| A-272 | CH(CH₃)—c-C₃H₅ | CH₂C≡CH |
| A-273 | CH₂—c-C₅H₉ | CH₂C≡CH |
| A-274 | CH₂—c-C₆H₁₁ | CH₂C≡CH |
| A-275 | C₆H₅ | CH₂C≡CH |
| A-276 | CH₃ | CH(CH₃)CH═CH₂ |
| A-277 | C₂H₅ | CH(CH₃)CH═CH₂ |
| A-278 | CH═CH₂ | CH(CH₃)CH═CH₂ |
| A-279 | CH₂CH₂CH₃ | CH(CH₃)CH═CH₂ |
| A-280 | CH(CH₃)₂ | CH(CH₃)CH═CH₂ |
| A-281 | CH₂CH₂CH₂CH₃ | CH(CH₃)CH═CH₂ |
| A-282 | C(CH₃)₃ | CH(CH₃)CH═CH₂ |
| A-283 | CH₂CH(CH₃)₂ | CH(CH₃)CH═CH₂ |
| A-284 | CH(CH₃)CH₂CH₃ | CH(CH₃)CH═CH₂ |
| A-285 | CH₂CH═CH₂ | CH(CH₃)CH═CH₂ |
| A-286 | CH₂C≡CH | CH(CH₃)CH═CH₂ |
| A-287 | CH(CH₃)CH═CH₂ | CH(CH₃)CH═CH₂ |
| A-288 | CHF₂ | CH(CH₃)CH═CH₂ |
| A-289 | CH₂Cl | CH(CH₃)CH═CH₂ |
| A-290 | CH₂CH₂CN | CH(CH₃)CH═CH₂ |
| A-291 | CH₂CH₂Cl | CH(CH₃)CH═CH₂ |
| A-292 | c-C₃H₅ | CH(CH₃)CH═CH₂ |
| A-293 | c-C₄H₇ | CH(CH₃)CH═CH₂ |
| A-294 | c-C₅H₉ | CH(CH₃)CH═CH₂ |
| A-295 | c-C₆H₁₁ | CH(CH₃)CH═CH₂ |
| A-296 | CH₂—c-C₃H₅ | CH(CH₃)CH═CH₂ |
| A-297 | CH(CH₃)—c-C₃H₅ | CH(CH₃)CH═CH₂ |
| A-298 | CH₂—c-C₅H₉ | CH(CH₃)CH═CH₂ |
| A-299 | CH₂—c-C₅H₉ | CH(CH₃)CH═CH₂ |
| A-300 | C₆H₅ | CH(CH₃)CH═CH₂ |
| A-301 | CH₃ | CHF₂ |
| A-302 | C₂H₅ | CHF₂ |
| A-303 | CH═CH₂ | CHF₂ |
| A-304 | CH₂CH₂CH₃ | CHF₂ |
| A-305 | CH(CH₃)₂ | CHF₂ |
| A-306 | CH₂CH₂CH₂CH₃ | CHF₂ |
| A-307 | C(CH₃)₃ | CHF₂ |
| A-308 | CH₂CH(CH₃)₂ | CHF₂ |
| A-309 | CH(CH₃)CH₂CH₃ | CHF₂ |
| A-310 | CH₂CH═CH₂ | CHF₂ |
| A-311 | CH₂C≡CH | CHF₂ |
| A-312 | CH(CH₃)CH═CH₂ | CHF₂ |
| A-313 | CHF₂ | CHF₂ |
| A-314 | CH₂Cl | CHF₂ |
| A-315 | CH₂CH₂CN | CHF₂ |
| A-316 | CH₂CH₂Cl | CHF₂ |
| A-317 | c-C₃H₅ | CHF₂ |
| A-318 | c-C₄H₇ | CHF₂ |
| A-319 | c-C₅H₉ | CHF₂ |
| A-320 | c-C₆H₁₁ | CHF₂ |
| A-321 | CH₂—c-C₃H₅ | CHF₂ |
| A-322 | CH(CH₃)—c-C₃H₅ | CHF₂ |
| A-323 | CH₂—c-C₅H₉ | CHF₂ |
| A-324 | CH₂—c-C₆H₁₁ | CHF₂ |
| A-325 | C₆H₅ | CHF₂ |
| A-326 | CH₃ | CH₂Cl |
| A-327 | C₂H₅ | CH₂Cl |
| A-328 | CH═CH₂ | CH₂Cl |
| A-329 | CH₂CH₂CH₃ | CH₂Cl |
| A-330 | CH(CH₃)₂ | CH₂Cl |
| A-331 | CH₂CH₂CH₂CH₃ | CH₂Cl |
| A-332 | C(CH₃)₃ | CH₂Cl |
| A-333 | CH₂CH(CH₃)₂ | CH₂Cl |
| A-334 | CH(CH₃)CH₂CH₃ | CH₂Cl |
| A-335 | CH₂CH═CH₂ | CH₂Cl |
| A-336 | CH₂C≡CH | CH₂Cl |
| A-337 | CH(CH₃)CH═CH₂ | CH₂Cl |
| A-338 | CHF₂ | CH₂Cl |
| A-339 | CH₂Cl | CH₂Cl |
| A-340 | CH₂CH₂CN | CH₂Cl |
| A-341 | CH₂CH₂Cl | CH₂Cl |
| A-342 | c-C₃H₅ | CH₂Cl |
| A-343 | c-C₄H₇ | CH₂Cl |
| A-344 | c-C₅H₉ | CH₂Cl |
| A-345 | c-C₆H₁₁ | CH₂Cl |
| A-346 | CH₂—c-C₃H₅ | CH₂Cl |
| A-347 | CH(CH₃)—c-C₃H₅ | CH₂Cl |
| A-348 | CH₂—c-C₅H₉ | CH₂Cl |
| A-349 | CH₂—c-C₆H₁₁ | CH₂Cl |
| A-350 | C₆H₅ | CH₂Cl |
| A-351 | CH₃ | CH₂CH₂CN |
| A-352 | C₂H₅ | CH₂CH₂CN |
| A-353 | CH═CH₂ | CH₂CH₂CN |
| A-354 | CH₂CH₂CH₃ | CH₂CH₂CN |
| A-355 | CH(CH₃)₂ | CH₂CH₂CN |
| A-356 | CH₂CH₂CH₂CH₃ | CH₂CH₂CN |
| A-357 | C(CH₃)₃ | CH₂CH₂CN |
| A-358 | CH₂CH(CH₃)₂ | CH₂CH₂CN |
| A-359 | CH(CH₃)CH₂CH₃ | CH₂CH₂CN |
| A-360 | CH₂CH═CH₂ | CH₂CH₂CN |
| A-361 | CH₂C≡CH | CH₂CH₂CN |
| A-362 | CH(CH₃)CH═CH₂ | CH₂CH₂CN |
| A-363 | CHF₂ | CH₂CH₂CN |
| A-364 | CH₂Cl | CH₂CH₂CN |
| A-365 | CH₂CH₂CN | CH₂CH₂CN |
| A-366 | CH₂CH₂Cl | CH₂CH₂CN |
| A-367 | c-C₃H₅ | CH₂CH₂CN |
| A-368 | c-C₄H₇ | CH₂CH₂CN |
| A-369 | c-C₅H₉ | CH₂CH₂CN |
| A-370 | c-C₆H₁₁ | CH₂CH₂CN |
| A-371 | CH₂—c-C₃H₅ | CH₂CH₂CN |
| A-372 | CH(CH₃)—c-C₃H₅ | CH₂CH₂CN |
| A-373 | CH₂—c-C₅H₉ | CH₂CH₂CN |
| A-374 | CH₂—c-C₅H₉ | CH₂CH₂CN |
| A-375 | C₆H₅ | CH₂CH₂CN |
| A-376 | CH₃ | CH₂CH₂Cl |
| A-377 | C₂H₅ | CH₂CH₂Cl |
| A-378 | CH═CH₂ | CH₂CH₂Cl |
| A-379 | CH₂CH₂CH₃ | CH₂CH₂Cl |
| A-380 | CH(CH₃)₂ | CH₂CH₂Cl |
| A-381 | CH₂CH₂CH₂CH₃ | CH₂CH₂Cl |
| A-382 | C(CH₃)₃ | CH₂CH₂Cl |
| A-383 | CH₂CH(CH₃)₂ | CH₂CH₂Cl |
| A-384 | CH(CH₃)CH₂CH₃ | CH₂CH₂Cl |
| A-385 | CH₂CH═CH₂ | CH₂CH₂Cl |
| A-386 | CH₂C≡CH | CH₂CH₂Cl |
| A-387 | CH(CH₃)CH═CH₂ | CH₂CH₂Cl |
| A-388 | CHF₂ | CH₂CH₂Cl |
| A-389 | CH₂Cl | CH₂CH₂Cl |
| A-390 | CH₂CH₂CN | CH₂CH₂Cl |
| A-391 | CH₂CH₂Cl | CH₂CH₂Cl |
| A-392 | c-C₃H₅ | CH₂CH₂Cl |
| A-393 | c-C₄H₇ | CH₂CH₂Cl |
| A-394 | c-C₅H₉ | CH₂CH₂Cl |
| A-395 | c-C₆H₁₁ | CH₂CH₂Cl |
| A-396 | CH₂—c-C₃H₅ | CH₂CH₂Cl |
| A-397 | CH(CH₃)—c-C₃H₅ | CH₂CH₂Cl |
| A-398 | CH₂—c-C₅H₉ | CH₂CH₂Cl |
| A-399 | CH₂—c-C₆H₁₁ | CH₂CH₂Cl |
| A-400 | C₆H₅ | CH₂CH₂Cl |
| A-401 | CH₃ | c-C₃H₅ |
| A-402 | C₂H₅ | c-C₃H₅ |
| A-403 | CH═CH₂ | c-C₃H₅ |
| A-404 | CH₂CH₂CH₃ | c-C₃H₅ |
| A-405 | CH(CH₃)₂ | c-C₃H₅ |
| A-406 | CH₂CH₂CH₂CH₃ | c-C₃H₅ |
| A-407 | C(CH₃)₃ | c-C₃H₅ |
| A-408 | CH₂CH(CH₃)₂ | c-C₃H₅ |
| A-409 | CH(CH₃)CH₂CH₃ | c-C₃H₅ |
| A-410 | CH₂CH═CH₂ | c-C₃H₅ |
| A-411 | CH₂C≡CH | c-C₃H₅ |
| A-412 | CH(CH₃)CH═CH₂ | c-C₃H₅ |
| A-413 | CHF₂ | c-C₃H₅ |
| A-414 | CH₂Cl | c-C₃H₅ |
| A-415 | CH₂CH₂CN | c-C₃H₅ |
| A-416 | CH₂CH₂Cl | c-C₃H₅ |
| A-417 | c-C₃H₅ | c-C₃H₅ |
| A-418 | c-C₄H₇ | c-C₃H₅ |
| A-419 | c-C₅H₉ | c-C₃H₅ |
| A-420 | c-C₆H₁₁ | c-C₃H₅ |
| A-421 | CH₂—c-C₃H₅ | c-C₃H₅ |
| A-422 | CH(CH₃)—c-C₃H₅ | c-C₃H₅ |
| A-423 | CH₂—c-C₅H₉ | c-C₃H₅ |
| A-424 | CH₂—c-C₆H₁₁ | c-C₃H₅ |
| A-425 | C₆H₅ | c-C₃H₅ |
| A-426 | CH₃ | c-C₄H₇ |
| A-427 | C₂H₅ | c-C₄H₇ |
| A-428 | CH═CH₂ | c-C₄H₇ |
| A-429 | CH₂CH₂CH₃ | c-C₄H₇ |
| A-430 | CH(CH₃)₂ | c-C₄H₇ |
| A-431 | CH₂CH₂CH₂CH₃ | c-C₄H₇ |
| A-432 | C(CH₃)₃ | c-C₄H₇ |
| A-433 | CH₂CH(CH₃)₂ | c-C₄H₇ |
| A-434 | CH(CH₃)CH₂CH₃ | c-C₄H₇ |
| A-435 | CH₂CH═CH₂ | c-C₄H₇ |
| A-436 | CH₂C≡CH | c-C₄H₇ |
| A-437 | CH(CH₃)CH═CH₂ | c-C₄H₇ |
| A-438 | CHF₂ | c-C₄H₇ |
| A-439 | CH₂Cl | c-C₄H₇ |
| A-440 | CH₂CH₂CN | c-C₄H₇ |
| A-441 | CH₂CH₂Cl | c-C₄H₇ |
| A-442 | c-C₃H₅ | c-C₄H₇ |
| A-443 | c-C₄H₇ | c-C₄H₇ |
| A-444 | c-C₅H₉ | c-C₄H₇ |
| A-445 | c-C₆H₁₁ | c-C₄H₇ |
| A-446 | CH₂—c-C₃H₅ | c-C₄H₇ |
| A-447 | CH(CH₃)—c-C₃H₅ | c-C₄H₇ |
| A-448 | CH₂—c-C₅H₉ | c-C₄H₇ |
| A-449 | CH₂—c-C₆H₁₁ | c-C₄H₇ |
| A-450 | C₆H₅ | c-C₄H₇ |
| A-451 | CH₃ | c-C₅H₉ |
| A-452 | C₂H₅ | c-C₅H₉ |
| A-453 | CH═CH₂ | c-C₅H₉ |
| A-454 | CH₂CH₂CH₃ | c-C₅H₉ |
| A-455 | CH(CH₃)₂ | c-C₅H₉ |
| A-456 | CH₂CH₂CH₂CH₃ | c-C₅H₉ |
| A-457 | C(CH₃)₃ | c-C₅H₉ |
| A-458 | CH₂CH(CH₃)₂ | c-C₅H₉ |
| A-459 | CH(CH₃)CH₂CH₃ | c-C₅H₉ |
| A-460 | CH₂CH═CH₂ | c-C₅H₉ |
| A-461 | CH₂C≡CH | c-C₅H₉ |
| A-462 | CH(CH₃)CH═CH₂ | c-C₅H₉ |
| A-463 | CHF₂ | c-C₅H₉ |
| A-464 | CH₂Cl | c-C₅H₉ |
| A-465 | CH₂CH₂CN | c-C₅H₉ |
| A-466 | CH₂CH₂Cl | c-C₅H₉ |
| A-467 | c-C₃H₅ | c-C₅H₉ |
| A-468 | c-C₄H₇ | c-C₅H₉ |
| A-469 | c-C₅H₉ | c-C₅H₉ |
| A-470 | c-C₆H₁₁ | c-C₅H₉ |
| A-471 | CH₂—c-C₃H₅ | c-C₅H₉ |
| A-472 | CH(CH₃)—c-C₃H₅ | c-C₅H₉ |
| A-473 | CH₂—c-C₅H₉ | c-C₅H₉ |
| A-474 | CH₂—c-C₆H₁₁ | c-C₅H₉ |
| A-475 | C₆H₅ | c-C₅H₉ |
| A-476 | CH₃ | c-C₆H₁₁ |
| A-477 | C₂H₅ | c-C₆H₁₁ |
| A-478 | CH═CH₂ | c-C₆H₁₁ |
| A-479 | CH₂CH₂CH₃ | c-C₆H₁₁ |
| A-480 | CH(CH₃)₂ | c-C₆H₁₁ |
| A-481 | CH₂CH₂CH₂CH₃ | c-C₆H₁₁ |
| A-482 | C(CH₃)₃ | c-C₆H₁₁ |
| A-483 | CH₂CH(CH₃)₂ | c-C₆H₁₁ |
| A-484 | CH(CH₃)CH₂CH₃ | c-C₆H₁₁ |
| A-485 | CH₂CH═CH₂ | c-C₆H₁₁ |
| A-486 | CH₂C≡CH | c-C₆H₁₁ |
| A-487 | CH(CH₃)CH═CH₂ | c-C₆H₁₁ |
| A-488 | CHF₂ | c-C₆H₁₁ |
| A-489 | CH₂Cl | c-C₆H₁₁ |
| A-490 | CH₂CH₂CN | c-C₆H₁₁ |
| A-491 | CH₂CH₂Cl | c-C₆H₁₁ |
| A-492 | c-C₃H₅ | c-C₆H₁₁ |
| A-493 | c-C₄H₇ | c-C₆H₁₁ |
| A-494 | c-C₅H₉ | c-C₆H₁₁ |
| A-495 | c-C₆H₁₁ | c-C₆H₁₁ |
| A-496 | CH₂—c-C₃H₅ | c-C₆H₁₁ |
| A-497 | CH(CH₃)—c-C₃H₅ | c-C₆H₁₁ |
| A-498 | CH₂—c-C₅H₉ | c-C₆H₁₁ |
| A-499 | CH₂—c-C₆H₁₁ | c-C₆H₁₁ |
| A-500 | C₆H₅ | c-C₆H₁₁ |
| A-501 | CH₃ | CH₂—c-C₃H₅ |
| A-502 | C₂H₅ | CH₂—c-C₃H₅ |
| A-503 | CH═CH₂ | CH₂—c-C₃H₅ |
| A-504 | CH₂CH₂CH₃ | CH₂—c-C₃H₅ |
| A-505 | CH(CH₃)₂ | CH₂—c-C₃H₅ |
| A-506 | CH₂CH₂CH₂CH₃ | CH₂—c-C₃H₅ |
| A-507 | C(CH₃)₃ | CH₂—c-C₃H₅ |
| A-508 | CH₂CH(CH₃)₂ | CH₂—c-C₃H₅ |
| A-509 | CH(CH₃)CH₂CH₃ | CH₂—c-C₃H₅ |
| A-510 | CH₂CH═CH₂ | CH₂—c-C₃H₅ |
| A-511 | CH₂C≡CH | CH₂—c-C₃H₅ |
| A-512 | CH(CH₃)CH═CH₂ | CH₂—c-C₃H₅ |
| A-513 | CHF₂ | CH₂—c-C₃H₅ |
| A-514 | CH₂Cl | CH₂—c-C₃H₅ |
| A-515 | CH₂CH₂CN | CH₂—c-C₃H₅ |
| A-516 | CH₂CH₂Cl | CH₂—c-C₃H₅ |
| A-517 | c-C₃H₅ | CH₂—c-C₃H₅ |
| A-518 | c-C₄H₇ | CH₂—c-C₃H₅ |
| A-519 | c-C₅H₉ | CH₂—c-C₃H₅ |
| A-520 | c-C₆H₁₁ | CH₂—c-C₃H₅ |
| A-521 | CH₂—c-C₃H₅ | CH₂—c-C₃H₅ |
| A-522 | CH(CH₃)—c-C₃H₅ | CH₂—c-C₃H₅ |
| A-523 | CH₂—c-C₅H₉ | CH₂—c-C₃H₅ |
| A-524 | CH₂—c-C₆H₁₁ | CH₂—c-C₃H₅ |
| A-525 | C₆H₅ | CH₂—c-C₃H₅ |
| A-526 | CH₃ | CH(CH₃)—c-C₃H₅ |
| A-527 | C₂H₅ | CH(CH₃)—c-C₃H₅ |
| A-528 | CH═CH₂ | CH(CH₃)—c-C₃H₅ |
| A-529 | CH₂CH₂CH₃ | CH(CH₃)—c-C₃H₅ |
| A-530 | CH(CH₃)₂ | CH(CH₃)—c-C₃H₅ |
| A-531 | CH₂CH₂CH₂CH₃ | CH(CH₃)—c-C₃H₅ |
| A-532 | C(CH₃)₃ | CH(CH₃)—c-C₃H₅ |
| A-533 | CH₂CH(CH₃)₂ | CH(CH₃)—c-C₃H₅ |
| A-534 | CH(CH₃)CH₂CH₃ | CH(CH₃)—c-C₃H₅ |
| A-535 | CH₂CH═CH₂ | CH(CH₃)—c-C₃H₅ |
| A-536 | CH₂C≡CH | CH(CH₃)—c-C₃H₅ |
| A-537 | CH(CH₃)CH═CH₂ | CH(CH₃)—c-C₃H₅ |
| A-538 | CHF₂ | CH(CH₃)—c-C₃H₅ |
| A-539 | CH₂Cl | CH(CH₃)—c-C₃H₅ |
| A-540 | CH₂CH₂CN | CH(CH₃)—c-C₃H₅ |
| A-541 | CH₂CH₂Cl | CH(CH₃)—c-C₃H₅ |
| A-542 | c-C₃H₅ | CH(CH₃)—c-C₃H₅ |
| A-543 | c-C₄H₇ | CH(CH₃)—c-C₃H₅ |
| A-544 | c-C₅H₉ | CH(CH₃)—c-C₃H₅ |
| A-545 | c-C₆H₁₁ | CH(CH₃)—c-C₃H₅ |
| A-546 | CH₂—c-C₃H₅ | CH(CH₃)—c-C₃H₅ |
| A-547 | CH(CH₃)—c-C₃H₅ | CH(CH₃)—c-C₃H₅ |
| A-548 | CH₂—c-C₅H₉ | CH(CH₃)—c-C₃H₅ |
| A-549 | CH₂—c-C₆H₁₁ | CH(CH₃)—c-C₃H₅ |
| A-550 | C₆H₅ | CH(CH₃)—c-C₃H₅ |
| A-551 | CH₃ | CH₂—c-C₅H₉ |
| A-552 | C₂H₅ | CH₂—c-C₅H₉ |
| A-553 | CH═CH₂ | CH₂—c-C₅H₉ |
| A-554 | CH₂CH₂CH₃ | CH₂—c-C₅H₉ |
| A-555 | CH(CH₃)₂ | CH₂—c-C₅H₉ |
| A-556 | CH₂CH₂CH₂CH₃ | CH₂—c-C₅H₉ |
| A-557 | C(CH₃)₃ | CH₂—c-C₅H₉ |
| A-558 | CH₂CH(CH₃)₂ | CH₂—c-C₅H₉ |
| A-559 | CH(CH₃)CH₂CH₃ | CH₂—c-C₅H₉ |
| A-560 | CH₂CH═CH₂ | CH₂—c-C₅H₉ |
| A-561 | CH₂C≡CH | CH₂—c-C₅H₉ |
| A-562 | CH(CH₃)CH═CH₂ | CH₂—c-C₅H₉ |
| A-563 | CHF₂ | CH₂—c-C₅H₉ |
| A-564 | CH₂Cl | CH₂—c-C₅H₉ |
| A-565 | CH₂CH₂CN | CH₂—c-C₅H₉ |
| A-566 | CH₂CH₂Cl | CH₂—c-C₅H₉ |
| A-567 | c-C₃H₅ | CH₂—c-C₅H₉ |
| A-568 | c-C₄H₇ | CH₂—c-C₅H₉ |
| A-569 | c-C₅H₉ | CH₂—c-C₅H₉ |
| A-570 | c-C₆H₁₁ | CH₂—c-C₅H₉ |
| A-571 | CH₂—c-C₃H₅ | CH₂—c-C₅H₉ |
| A-572 | CH(CH₃)—c-C₃H₅ | CH₂—c-C₅H₉ |
| A-573 | CH₂—c-C₅H₉ | CH₂—c-C₅H₉ |
| A-574 | CH₂—c-C₆H₁₁ | CH₂—c-C₅H₉ |
| A-575 | C₆H₅ | CH₂—c-C₅H₉ |
| A-576 | CH₃ | CH₂—c-C₆H₁₁ |
| A-577 | C₂H₅ | CH₂—c-C₆H₁₁ |
| A-578 | CH═CH₂ | CH₂—c-C₆H₁₁ |
| A-579 | CH₂CH₂CH₃ | CH₂—c-C₆H₁₁ |
| A-580 | CH(CH₃)₂ | CH₂—c-C₆H₁₁ |
| A-581 | CH₂CH₂CH₂CH₃ | CH₂—c-C₆H₁₁ |
| A-582 | C(CH₃)₃ | CH₂—c-C₆H₁₁ |
| A-583 | CH₂CH(CH₃)₂ | CH₂—c-C₆H₁₁ |
| A-584 | CH(CH₃)CH₂CH₃ | CH₂—c-C₆H₁₁ |
| A-585 | CH₂CH═CH₂ | CH₂—c-C₆H₁₁ |
| A-586 | CH₂C≡CH | CH₂—c-C₆H₁₁ |
| A-587 | CH(CH₃)CH═CH₂ | CH₂—c-C₆H₁₁ |
| A-588 | CHF₂ | CH₂—c-C₆H₁₁ |
| A-589 | CH₂Cl | CH₂—c-C₆H₁₁ |
| A-590 | CH₂CH₂CN | CH₂—c-C₆H₁₁ |
| A-591 | CH₂CH₂Cl | CH₂—c-C₆H₁₁ |
| A-592 | c-C₃H₅ | CH₂—c-C₆H₁₁ |
| A-593 | c-C₄H₇ | CH₂—c-C₆H₁₁ |
| A-594 | c-C₅H₉ | CH₂—c-C₆H₁₁ |
| A-595 | c-C₆H₁₁ | CH₂—c-C₆H₁₁ |
| A-596 | CH₂—c-C₃H₅ | CH₂—c-C₆H₁₁ |
| A-597 | CH(CH₃)—c-C₃H₅ | CH₂—c-C₆H₁₁ |
| A-598 | CH₂—c-C₅H₉ | CH₂—c-C₆H₁₁ |
| A-599 | CH₂—c-C₆H₁₁ | CH₂—c-C₆H₁₁ |
| A-600 | C₆H₅ | CH₂—c-C₆H₁₁ |
| A-601 | CH₃ | C₆H₅ |
| A-602 | C₂H₅ | C₆H₅ |
| A-603 | CH═CH₂ | C₆H₅ |
| A-604 | CH₂CH₂CH₃ | C₆H₅ |
| A-605 | CH(CH₃)₂ | C₆H₅ |
| A-606 | CH₂CH₂CH₂CH₃ | C₆H₅ |
| A-607 | C(CH₃)₃ | C₆H₅ |
| A-608 | CH₂CH(CH₃)₂ | C₆H₅ |
| A-609 | CH(CH₃)CH₂CH₃ | C₆H₅ |
| A-610 | CH₂CH═CH₂ | C₆H₅ |
| A-611 | CH₂C≡CH | C₆H₅ |
| A-612 | CH(CH₃)CH═CH₂ | C₆H₅ |
| A-613 | CHF₂ | C₆H₅ |
| A-614 | CH₂Cl | C₆H₅ |
| A-615 | CH₂CH₂CN | C₆H₅ |
| A-616 | CH₂CH₂Cl | C₆H₅ |
| A-617 | c-C₃H₅ | C₆H₅ |
| A-618 | c-C₄H₇ | C₆H₅ |
| A-619 | c-C₅H₉ | C₆H₅ |
| A-620 | c-C₆H₁₁ | C₆H₅ |
| A-621 | CH₂—c-C₃H₅ | C₆H₅ |
| A-622 | CH(CH₃)—c-C₃H₅ | C₆H₅ |
| A-623 | CH₂—c-C₅H₉ | C₆H₅ |
| A-624 | CH₂—c-C₆H₁₁ | C₆H₅ |
| A-625 | C₆H₅ | C₆H₅ |
| A-626 | CH₃ | CH₂—c-C₄H₇ |
| A-627 | C₂H₅ | CH₂—c-C₄H₇ |
| A-628 | CH═CH₂ | CH₂—c-C₄H₇ |
| A-629 | CH₂CH₂CH₃ | CH₂—c-C₄H₇ |
| A-630 | CH(CH₃)₂ | CH₂—c-C₄H₇ |
| A-631 | CH₂CH₂CH₂CH₃ | CH₂—c-C₄H₇ |
| A-632 | C(CH₃)₃ | CH₂—c-C₄H₇ |
| A-633 | CH₂CH(CH₃)₂ | CH₂—c-C₄H₇ |
| A-634 | CH(CH₃)CH₂CH₃ | CH₂—c-C₄H₇ |
| A-635 | CH₂CH═CH₂ | CH₂—c-C₄H₇ |
| A-636 | CH₂C≡CH | CH₂—c-C₄H₇ |
| A-637 | CH(CH₃)CH═CH₂ | CH₂—c-C₄H₇ |
| A-638 | CHF₂ | CH₂—c-C₄H₇ |
| A-639 | CH₂Cl | CH₂—c-C₄H₇ |
| A-640 | CH₂CH₂CN | CH₂—c-C₄H₇ |
| A-641 | CH₂CH₂Cl | CH₂—c-C₄H₇ |
| A-642 | c-C₃H₅ | CH₂—c-C₄H₇ |
| A-643 | c-C₄H₇ | CH₂—c-C₄H₇ |
| A-644 | c-C₅H₉ | CH₂—c-C₄H₇ |
| A-645 | c-C₆H₁₁ | CH₂—c-C₄H₇ |
| A-646 | CH₂—c-C₃H₅ | CH₂—c-C₄H₇ |
| A-647 | CH(CH₃)—c-C₃H₅ | CH₂—c-C₄H₇ |
| A-648 | CH₂—c-C₅H₉ | CH₂—c-C₄H₇ |
| A-649 | CH₂—c-C₆H₁₁ | CH₂—c-C₄H₇ |
| A-650 | C₆H₅ | CH₂—c-C₄H₇ |
| A-651 | CH₃ | CH₂CH₂—c-C₃H₅ |
| A-652 | C₂H₅ | CH₂CH₂—c-C₃H₅ |
| A-653 | CH═CH₂ | CH₂CH₂—c-C₃H₅ |
| A-654 | CH₂CH₂CH₃ | CH₂CH₂—c-C₃H₅ |
| A-655 | CH(CH₃)₂ | CH₂CH₂—c-C₃H₅ |
| A-656 | CH₂CH₂CH₂CH₃ | CH₂CH₂—c-C₃H₅ |
| A-657 | C(CH₃)₃ | CH₂CH₂—c-C₃H₅ |
| A-658 | CH₂CH(CH₃)₂ | CH₂CH₂—c-C₃H₅ |
| A-659 | CH(CH₃)CH₂CH₃ | CH₂CH₂—c-C₃H₅ |
| A-660 | CH₂CH═CH₂ | CH₂CH₂—c-C₃H₅ |
| A-661 | CH₂C≡CH | CH₂CH₂—c-C₃H₅ |
| A-662 | CH(CH₃)CH═CH₂ | CH₂CH₂—c-C₃H₅ |
| A-663 | CHF₂ | CH₂CH₂—c-C₃H₅ |
| A-664 | CH₂Cl | CH₂CH₂—c-C₃H₅ |
| A-665 | CH₂CH₂CN | CH₂CH₂—c-C₃H₅ |
| A-666 | CH₂CH₂Cl | CH₂CH₂—c-C₃H₅ |
| A-667 | c-C₃H₅ | CH₂CH₂—c-C₃H₅ |
| A-668 | c-C₄H₇ | CH₂CH₂—c-C₃H₅ |
| A-669 | c-C₅H₉ | CH₂CH₂—c-C₃H₅ |
| A-670 | c-C₆H₁₁ | CH₂CH₂—c-C₃H₅ |
| A-671 | CH₂—c-C₃H₅ | CH₂CH₂—c-C₃H₅ |
| A-672 | CH(CH₃)—c-C₃H₅ | CH₂CH₂—c-C₃H₅ |
| A-673 | CH₂—c-C₅H₉ | CH₂CH₂—c-C₃H₅ |
| A-674 | CH₂—c-C₆H₁₁ | CH₂CH₂—c-C₃H₅ |
| A-675 | C₆H₅ | CH₂CH₂—c-C₃H₅ |
| A-676 | CH₃ | CH₂(CH₂)₃CH₃ |
| A-677 | C₂H₅ | CH₂(CH₂)₃CH₃ |
| A-678 | CH═CH₂ | CH₂(CH₂)₃CH₃ |
| A-679 | CH₂CH₂CH₃ | CH₂(CH₂)₃CH₃ |
| A-680 | CH(CH₃)₂ | CH₂(CH₂)₃CH₃ |
| A-681 | CH₂CH₂CH₂CH₃ | CH₂(CH₂)₃CH₃ |
| A-682 | C(CH₃)₃ | CH₂(CH₂)₃CH₃ |
| A-683 | CH₂CH(CH₃)₂ | CH₂(CH₂)₃CH₃ |
| A-684 | CH(CH₃)CH₂CH₃ | CH₂(CH₂)₃CH₃ |
| A-685 | CH₂CH═CH₂ | CH₂(CH₂)₃CH₃ |
| A-686 | CH₂C≡CH | CH₂(CH₂)₃CH₃ |
| A-687 | CH(CH₃)CH═CH₂ | CH₂(CH₂)₃CH₃ |
| A-688 | CHF₂ | CH₂(CH₂)₃CH₃ |
| A-689 | CH₂Cl | CH₂(CH₂)₃CH₃ |
| A-690 | CH₂CH₂CN | CH₂(CH₂)₃CH₃ |
| A-691 | CH₂CH₂Cl | CH₂(CH₂)₃CH₃ |
| A-692 | c-C₃H₅ | CH₂(CH₂)₃CH₃ |
| A-693 | c-C₄H₇ | CH₂(CH₂)₃CH₃ |
| A-694 | c-C₅H₉ | CH₂(CH₂)₃CH₃ |
| A-695 | c-C₆H₁₁ | CH₂(CH₂)₃CH₃ |
| A-696 | CH₂—c-C₃H₅ | CH₂(CH₂)₃CH₃ |
| A-697 | CH(CH₃)—c-C₃H₅ | CH₂(CH₂)₃CH₃ |
| A-698 | CH₂—c-C₅H₉ | CH₂(CH₂)₃CH₃ |
| A-699 | CH₂—c-C₆H₁₁ | CH₂(CH₂)₃CH₃ |
| A-700 | C₆H₅ | CH₂(CH₂)₃CH₃ |
| A-701 | CH₃ | CH(CH₃)CH(CH₃)₂ |
| A-702 | C₂H₅ | CH(CH₃)CH(CH₃)₂ |
| A-703 | CH═CH₂ | CH(CH₃)CH(CH₃)₂ |
| A-704 | CH₂CH₂CH₃ | CH(CH₃)CH(CH₃)₂ |
| A-705 | CH(CH₃)₂ | CH(CH₃)CH(CH₃)₂ |
| A-706 | CH₂CH₂CH₂CH₃ | CH(CH₃)CH(CH₃)₂ |
| A-707 | C(CH₃)₃ | CH(CH₃)CH(CH₃)₂ |
| A-708 | CH₂CH(CH₃)₂ | CH(CH₃)CH(CH₃)₂ |
| A-709 | CH(CH₃)CH₂CH₃ | CH(CH₃)CH(CH₃)₂ |
| A-710 | CH₂CH═CH₂ | CH(CH₃)CH(CH₃)₂ |
| A-711 | CH₂C≡CH | CH(CH₃)CH(CH₃)₂ |
| A-712 | CH(CH₃)CH═CH₂ | CH(CH₃)CH(CH₃)₂ |
| A-713 | CHF₂ | CH(CH₃)CH(CH₃)₂ |
| A-714 | CH₂Cl | CH(CH₃)CH(CH₃)₂ |
| A-715 | CH₂CH₂CN | CH(CH₃)CH(CH₃)₂ |
| A-716 | CH₂CH₂Cl | CH(CH₃)CH(CH₃)₂ |
| A-717 | c-C₃H₅ | CH(CH₃)CH(CH₃)₂ |
| A-718 | c-C₄H₇ | CH(CH₃)CH(CH₃)₂ |
| A-719 | c-C₅H₉ | CH(CH₃)CH(CH₃)₂ |
| A-720 | c-C₆H₁₁ | CH(CH₃)CH(CH₃)₂ |
| A-721 | CH₂—c-C₃H₅ | CH(CH₃)CH(CH₃)₂ |
| A-722 | CH(CH₃)—c-C₃H₅ | CH(CH₃)CH(CH₃)₂ |
| A-723 | CH₂—c-C₅H₉ | CH(CH₃)CH(CH₃)₂ |
| A-724 | CH₂—c-C₆H₁₁ | CH(CH₃)CH(CH₃)₂ |
| A-725 | C₆H₅ | CH(CH₃)CH(CH₃)₂ |
| A-726 | CH₃ | CH₂(CH₂)₄CH₃ |
| A-727 | C₂H₅ | CH₂(CH₂)₄CH₃ |
| A-728 | C(CH₃)₃ | CH₂(CH₂)₄CH₃ |
| A-729 | CH₂(CH₂)₄CH₃ | CH₂(CH₂)₄CH₃ |
| A-730 | CH₃ | 2-EtHex |
| A-731 | C₂H₅ | 2-EtHex |
| A-732 | C(CH₃)₃ | 2-EtHex |
| A-733 | 2-EtHex | 2-EtHex |
| A-734 | CH₃ | CH₂CH₂OH |
| A-735 | C₂H₅ | CH₂CH₂OH |
| A-736 | C(CH₃)₃ | CH₂CH₂OH |
| A-737 | CH₂CH₂CH₂CH₃ | CH₂CH₂OH |
| A-738 | CH₂(CH₂)₃CH₃ | CH₂CH₂OH |
| A-739 | CH₂CH₂OH | CH₂CH₂OH |
| A-740 | CH₂—c-C₄H₇ | CH₂—c-C₄H₇ |
| A-741 | CH₂CH₂—c-C₃H₅ | CH₂CH₂—c-C₃H₅ |
| A-742 | CH(CH₃)CH(CH₃)₂ | CH(CH₃)CH(CH₃)₂ |
| A-743 | CH₂(CH₂)₃CH₃ | CH₂(CH₂)₃CH₃ |

| | |
|---|---|
| A-744 | (CH₂)₄ |
| A-745 | CH₂CH₂SCH₂ |

c-C₃H₅: cyclopropyl; c-C₄H₇: cyclobutyl; c-C₅H₉: cyclopentyl; c-C₆H₁₁: cyclohexyl; CH₂-c-C₃H₅: cyclopropylmethyl; CH(CH₃)-c-C₃H₅: 1-cyclopropylethyl; CH₂-c-C₅H₉: cyclopentylmethyl; CH₂-c-C₅H₉: cyclopentylmethyl; C₆H₅: phenyl; CH₂CH₂-c-C₃H₅: 2-cyclopropylethyl; CH₂-c-C₄H₇: 2-cyclobutylmethyl; 2-EtHex: CH₂CH(C₂H₅)(CH₂)₃CH₃

A group of especially preferred compounds of formula I are compounds I-1 to I-40 of formula IA-1 which are listed in the table C in the example section.

In one embodiment, a compound selected from the compounds I-1 to I-40 as defined in Table C in the Example Section at the end of the description, are preferred in the methods and uses according to the invention.

In one embodiment, a compound selected from compounds I-11, I-16, I-21, I-26, I-31 is the compound I in the methods and uses according to the invention, which are defined in accordance with Table C of the example section:

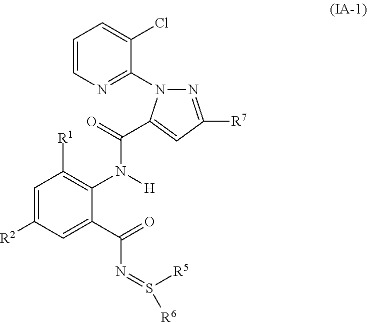

TABLE C′

| | R¹ | R² | R⁷ | R⁵ | R⁶ |
|---|---|---|---|---|---|
| I-11 | Me | Cl | CF₃ | C₂H₅ | C₂H₅ |
| I-16 | Cl | Cl | CF₃ | C₂H₅ | C₂H₅ |
| I-21 | Me | Cl | CF₃ | CH(CH₃)₂ | CH(CH₃)₂ |
| I-26 | Cl | Cl | CF₃ | CH(CH₃)₂ | CH(CH₃)₂ |
| I-31 | Br | Br | CF₃ | C₂H₅ | C₂H₅ |

In one embodiment, I-11 is the compound I in the methods and uses according to the invention.

In one embodiment, I-16 is the compound I in the methods and uses according to the invention.

In one embodiment, I-21 is the compound I in the methods and uses according to the invention.

In one embodiment, I-26 is the compound I in the methods and uses according to the invention.

In one embodiment, I-31 is the compound I in the methods and uses according to the invention.

Ryanodine-modulator insecticide resistant insects and methods for controlling them

Resistance may be defined as ‘a heritable change in the sensitivity of a pest population that is reflected in the repeated failure of a product to achieve the expected level of control when used according to the label recommendation for that pest species’. (IRAC) Cross-resistance occurs when resistance to one insecticide confers resistance to another insecticide via the same biochemical mechanism. This can happen within insecticide chemical groups or between insecticide chemical groups. Cross-resistance may occur even if the resistant insect has never been exposed to one of the chemical classes of insecticide.

Resistance therefore means that the original activitiy of a pesticide against the target organisms (arthropods, insects) decreases or is even lost, due to genetic adaptation of the target organism.

“Resistant” to a ryanodine-modulator insecticide is understood to mean resistant to at least one ryanodine-modulator insecticide, i.e. the insect may be resistant to only one, but also to several ryanodine-modulator insecticides.

The resistance may be also against an insecticidal effect which is due to a genetic modification of a plant (modified or transgenic plant), which caused a resistance of the plant or crop to certain pests, especially insect pests, in susceptible insects.

This is to be understood to include plants that are by the use of recombinant DNA techniques capable to synthesize one or more insecticidal proteins, especially those mentioned herein, especially those known from the bacterial genus Bacillus, particularly from Bacillus thuringiensis, such as endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, for example Photorhabdus spp. or Xenorhabdus spp., and so on.

Methods and uses of the invention as described herein may also involve a step of assessing whether insects are resistant to certain ryanodine-modulator insecticides. This step will in general involve collecting a sample of insects from the area (e.g. crop, field, habitat) to be treated, before actually applying a compound of formula I, and testing (for example using any suitable phenotypic, biochemical or molecular biological technique applicable) for resistance/sensitivity.

Ryanodine-modulator Insecticides to which the arthropods or insects may be resistant, in the sense of the methods and uses according to the invention, are

M.26 Ryanodine receptor-modulators from the class of diamides, as for example flubendiamide, chlorantraniliprole (rynaxypyr®), cyantraniliprole (cyazypyr®), or the phthalamide compounds

M.26.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and

M.26.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid,

or the anthranilamide compounds

M.26.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound

M.26.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a com-pound selected from M.26.5a) to M.26.5d):

M.26.5a: N-[2-(5-amino-1,3,4-thiadiazol-2-yl)-4-chloro-6-methyl-phenyl]-5-bromo-2-(3-chloro-2-pyridyl)pyrazole-3-carboxamide;

M.26.5b: 5-chloro-2-(3-chloro-2-pyridyl)-N-[2,4-dichloro-6-[(1-cyano-1-methyl-ethyl)carbamoyl]phenyl]pyrazole-3-carboxamide;

M.26.5c: 5-bromo-N-[2,4-dichloro-6-(methylcarbamoyl)phenyl]-2-(3,5-dichloro-2-pyridyl)pyrazole-3-carboxamide;

M.26.5d: N-[2-(tert-butylcarbamoyl)-4-chloro-6-methyl-phenyl]-2-(3-chloro-2-pyridyl)-5-(fluoromethoxy)pyrazole-3-carboxamide; or

M.26.6: N2-(1-cyano-1-methyl-ethyl)-N1-(2,4-dimethylphenyl)-3-iodo-phthalamide; or

M.26.7: 3-chloro-N2-(1-cyano-1-methyl-ethyl)-N1-(2,4-dimethylphenyl)phthalamide.

The commercially available compounds listed above may be found in The Pesticide Manual, 15th Edition, C. D. S. Tomlin, British Crop Protection Council (2011) among other publications. Cyantraniliprole (Cyazypyr) is known from e.g. WO 2004/067528. The phthalamides M.26.1 and M.26.2 are both known from WO 2007/101540. The anthranilamide M.26.3 has been described in WO 2005/077934. The hydrazide compound M.26.4 has been described in WO 2007/043677. The anthranilamide M.26.5a) is described in WO2011/085575, the M.26.5b) in WO2008/134969, the M.26.5c) in US2011/046186 and the M.26.5d in WO2012/034403. The diamide compounds M.26.6 and M.26.7 can be found in CN102613183.

In one embodiment, the Ryanodine-modulator Insecticide is Chlorantraniliprole.

In one embodiment, the Ryanodine-modulator Insecticide is Cyantraniliprole.

In one embodiment, the Ryanodine-modulator Insecticide is Cyclaniliprole.

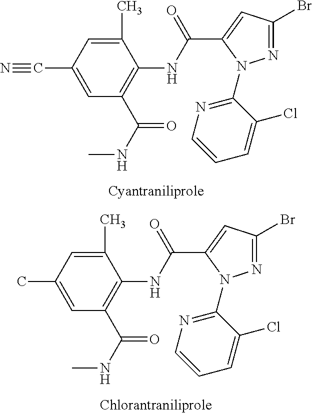 [SMILES: CNC(=O)c1cc(C#N)cc(C)c1NC(=O)c1cc(Br)nn1-c1ncccc1Cl ; CNC(=O)c1cc(C)cc(C)c1NC(=O)c1cc(Br)nn1-c1ncccc1Cl]

In a preferred embodiment, the method according to the invention is a method of controlling insects, which are resistant to a ryanodine-modulator insecticide, which method comprises applying to said ryanodine-modulator insecticide resistant insects at least one pesticidally active anthranilamide compound of formula (I), wherein the ryanodine-modulator insecticide to which the insect is resistant is selected from chlorantraniliprole or cyantraniliprole.

By the terms “control” or “controlling” as applied to insects, it is meant that the targeted insects are repelled from or less attracted to the crops to be protected. Additionally, as applied to insects, the terms “control” or “controlling” may also refer to the inability, or reduced ability, of the insects to feed or lay eggs. These terms may further include that the targeted insects are killed.

Thus the method of the invention may involve the use of an amount of the active ingredient that is sufficient to repel insects (i.e a repellently effective amount of active ingredient), an amount of the active ingredient that is sufficient to stop insects feeding, or it may involve the use of an insecticidally effective amount of active ingredient (i.e. an amount sufficient to kill insects), or any combination of the above effects.

The terms “applying” and “application” are understood to mean direct application to the insect to be controlled, as well as indirect application to said insect, for example through application to the crop or plant on which the insect acts as pest, or to the locus of said crop or insect, or indeed through treatment of the plant propagation material of said crop of plant.

Thus a compound of formula I may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the plant propagation material, such as seed, before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

In a further preferred embodiment of the invention, the invention relates to a method of protecting a crop of useful plants susceptible to and/or under attack by insects, which are resistant to a ryanodine-modulator insecticide, which method comprises applying to said crop, treating a plant propagation material of said crop with, and/or applying to said ryanodine-modulator insecticide resistant insects, a compound of formula I as defined herein.

In a further preferred embodiment of the invention, the invention relates to a method of controlling resistance to one or more ryanodine-modulator insecticides in insects, which comprises alternately applying a compound of formula I as defined herein, and the ryanodine-modulator insecticide, towards which the insects are resistant, to said insects or to a crop of useful plants susceptible to and/or under attack from said insects.

In these methods of the invention, the methods are also preferred wherein the ryanodine-modulator insecticide resistant insect is from the order Lepidoptera, Coleoptera or Diptera or is selected from thrips, hoppers and whitefly.

In these methods of the invention, preferred are also the methods of protecting a crop of useful plants susceptible to and/or under attack by insects, which insects are resistant to a ryanodine-modulator insecticide, and enhancing the crop, which method comprises applying to the propagation material of said crop an insecticide followed by the foliar application of a compound of the formula (I), as defined herein, beginning with the 3-to 5-leaf crop stage.

Pests

The compounds of the formula I, and their stereoisomers, salts, tautomers and N-oxides, are in particular suitable for efficiently controlling arthropodal pests such as arachnids, myriapedes and insects as well as nematodes, in the methods and uses according to the invention. The compounds of the formula I are especially suitable for efficiently combating the following pests in the methods and uses according to the invention:

insects from the order of the lepidopterans (Lepidoptera), for example Acronicta major, Adoxophyes orana, Aedia leucomelas, Agrotis spp. such as Agrotis fucosa, Agrotis segetum, Agrotis ypsilon; Alabama argillacea, Anticarsia gemmatalis, Anticarsia spp., Argyresthia conjugella, Autographa gamma, Barathra brassicae, Bucculatrix thurberiella, Bupalus piniarius, Cacoecia murinana, Cacoecia podana, Capua reticulana, Carpocapsa pomonella, Cheimatobia brumata, Chilo spp. such as Chilo suppressalis; Choristoneura fumiferana, Choristoneura occidentalis, Cirphis unipuncta, Clysia ambiguella, Cnaphalocerus spp., Cydia pomonella, Dendrolimus pini, Diaphania nitidalis, Diatraea grandiosella, Earias insulana, Elasmopalpus lignosellus, Ephestia cautella, Ephestia kuehniella, Eupoecilia ambiguella, Euproctis chrysorrhoea, Euxoa spp., Evetria bouliana, Feltia spp. such as Feltia subterranean; Galleria mellonella, Grapholitha funebrana, Grapholitha molesta, Helicoverpa spp. such as Helicoverpa armigera, Helicoverpa zea; Heliothis spp. such as Heliothis armigera, Heliothis virescens, Heliothis zea; Hellula undalis, Hibernia defoliaria, Hofmannophila pseudospretella, Homona magnanima, Hyphantria cunea, Hyponomeuta padella, Hyponomeuta malinellus, Keiferia lycopersicella, Lambdina fiscellaria, Laphygma spp. such as Laphygma exigua; Leucoptera coffeella, Leucoptera scitella, Lithocolletis blancardella, Lithophane antennata, Lobesia botrana, Loxagrotis albicosta, Loxostege sticticalis, Lymantria spp. such as Lymantria dispar, Lymantria monacha; Lyonetia clerkella, Malacosoma neustria, Mamestra spp. such as Mamestra brassicae; Mocis repanda, Mythimna separata, Orgyia pseudotsugata, Oria spp., Ostrinia spp. such as Ostrinia nubilalis; Oulema oryzae, Panolis flammea, Pectinophora spp. such as Pectinophora gossypiella; Peridroma saucia, Phalera bucephala, Phthorimaea spp. such as Phthorimaea operculella; Phyllocnistis citrella, Pieris spp. such as Pieris brassicae, Pieris rapae; Plathypena scabra, Plutella maculipennis, Plutella xylostella, Prodenia spp., Pseudaletia spp., Pseudoplusia includens, Pyrausta nubilalis, Rhyacionia frustrana, Scrobipalpula absoluta, Sitotroga cerealella, Sparganothis pilleriana, Spodoptera spp. such as Spodoptera frugiperda, Spodoptera littoralis, Spodoptera litura; Thaumatopoea pityocampa, Thermesia gemmatalis, Tinea pellionella, Tineola bisselliella, Tortrix viridana, Trichoplusia spp. such as Trichoplusia ni; Tuta absoluta, and Zeiraphera canadensis, beetles (Coleoptera), for example Acanthoscehdes obtectus, Adoretus spp., Agelastica alni, Agrilus sinuatus, Agriotes spp. such as Agriotes fuscicollis, Agriotes lineatus, Agriotes obscurus; Amphimallus solstitialis, Anisandrus dispar, Anobium punctatum, Anomala rufocuprea, Anoplophora spp. such as Anoplophora glabripennis; Anthonomus spp. such as Anthonomus grandis, Anthonomus pomorum; Anthrenus spp., Aphthona euphoridae, Apogonia spp., Athous haemorrhoidalis, Atomaria spp. such as Atomaria linearis; Attagenus spp., Aulacophora femoralis, Blastophagus piniperda, Blitophaga undata, Bruchidius obtectus, Bruchus spp. such as Bruchus lentis, Bruchus pisorum, Bruchus rufimanus; Byctiscus betulae, Callosobruchus chinensis, Cassida nebulosa, Cerotoma trifurcata, Cetonia aurata, Ceuthorhynchus spp. such as Ceuthorrhynchus assimilis, Ceuthorrhynchus napi; Chaetocnema tibialis, Cleonus mendicus, Conoderus spp. such as Conoderus vespertinus; Cosmopolites spp., Costelytra zealandica, Crioceris asparagi, Cryptorhynchus lapathi, Ctenicera ssp. such as Ctenicera destructor; Curculio spp., Dectes texanus, Dermestes spp., Diabrotica spp. such as Diabrotica 12-punctata Diabrotica speciosa, Diabrotica longicornis, Diabrotica semipunctata, Diabrotica virgifera; Epilachna spp. such as Epilachna varivestis, Epilachna vigintioctomaculata; Epitrix spp. such as Epitrix hirtipennis; Eutinobothrus brasiliensis, Faustinus cubae, Gibbium psylloides, Heteronychus arator, Hylamorpha elegans, Hylobius abietis, Hylotrupes bajulus, Hypera brunneipennis, Hypera postica, Hypothenemus spp., Ips typographus, Lachnosterna consanguinea, Lema bilineata, Lema melanopus, Leptinotarsa spp. such as Leptinotarsa decemlineata; Limonius californicus, Lissorhoptrus oryzophilus, Lissorhoptrus oryzophilus, Lixus spp., Lyctus spp. such as Lyctus bruneus; Melanotus communis, Meligethes spp. such as Meligethes aeneus; Melolontha hippocastani, Melolontha melolontha, Migdolus spp., Monochamus spp. such as Monochamus alternatus; Naupactus xanthographus, Niptus hololeucus, Oryctes rhinoceros, Oryzaephilus surinamensis, Otiorrhynchus sulcatus, Otiorrhynchus ovatus, Otiorrhynchus sulcatus, Oulema oryzae, Oxycetonia jucunda, Phaedon cochleariae, Phyllobius pyri, Phyllopertha horticola, Phyllophaga spp., Phyllotreta spp. such as Phyllotreta chrysocephala, Phyllotreta nemorum, Phyllotreta striolata; Phyllophaga spp., Phyllopertha horticola, Popillia japonica, Premnotrypes spp., Psylliodes chrysocephala, Ptinus spp., Rhizobius ventralis, Rhizopertha dominica, Sitona lineatus, Sitophilus spp. such as Sitophilus granaria, Sitophilus zeamais; Sphenophorus spp. such as Sphenophorus levis; Sternechus spp. such as Sternechus subsignatus; Symphyletes spp., Tenebrio molitor, Tribolium spp. such as Tribolium castaneum; Trogoderma spp., Tychius spp., Xylotrechus spp., and Zabrus spp. such as Zabrus tenebrioides,

flies, mosquitoes (Diptera), e.g. Aedes spp. such as Aedes aegypti, Aedes albopictus, Aedes vexans; Anastrepha ludens, Anopheles spp. such as Anopheles albimanus, Anopheles cruclans, Anopheles freeborni, Anopheles gambiae, Anopheles leucosphyrus, Anopheles maculipennis, Anopheles minimus, Anopheles quadrimaculatus, Anopheles sinensis; Bibio hortulanus, Calliphora erythrocephala, Calliphora vicina, Cerafitis capitata, Ceratitis capitata, Chrysomyia spp. such as Chrysomya bezziana, Chrysomya hominivorax, Chrysomya macellaria; Chrysops atlanticus, Chrysops discalis, Chrysops silacea, Cochliomyia spp. such as Cochliomyia hominivorax; Contarinia spp. such as Contarinia sorghicola; Cordylobia anthropophaga, Culex spp. such as Culex nigripalpus, Culex pipiens, Culex quinquefasciatus, Culex tarsalis, Culex tritaeniorhynchus; Culicoides furens, Culiseta inornata, Culiseta melanura, Cuterebra spp., Dacus cucurbitae, Dacus oleae, Dasineura brassicae, Delia spp. such as Delia antique, Delia coarctata, Delia platura, Delia radicum; Dermatobia hominis, Drosophila spp., Fannia spp. such as Fannia canicularis; Gastraphilus spp. such as Gasterophilus intestinalis; Geomyza Tripunctata, Glossina fuscipes, Glossina morsitans, Glossina palpalis, Glossina tachinoides, Haematobia irritans, Haplodiplosis equestris, Hippelates spp., Hylemyia spp. such as Hylemyia platura; Hypoderma spp. such as Hypoderma lineata; Hyppobosca spp., Leptoconops torrens, Liriomyza spp. such as Liriomyza sativae, Liriomyza trifolii; Lucilia spp. such as Lucilia caprina, Lucilia cuprina, Lucilia sericata; Lycoria pectoralis, Mansonia titillanus, Mayetiola spp. such as Mayetiola destructor; Musca spp. such as Musca autumnalis, Musca domestica; Muscina stabulans, Oestrus spp. such as Oestrus ovis; Opomyza florum, Oscinella spp. such as Oscinella frit; Pegomya hysocyami, Phlebotomus argentipes, Phorbia spp. such as Phorbia antiqua, Phorbia brassicae, Phorbia coarctata; Prosimulium mixtum, Psila rosae, Psorophora columbiae, Psorophora discolor, Rhagoletis cerasi, Rhagoletis pomonella, Sarcophaga spp. such as Sarcophaga haemorrhoidalis; Simulium vittatum, Stomoxys spp. such as Stomoxys calcitrans; Tabanus spp. such as Tabanus atratus, Tabanus bovinus, Tabanus lineola, Tabanus similis; Tannia spp., Tipula oleracea, Tipula paludosa, and Wohlfahrtia spp.,

thrips (Thysanoptera), e.g. Baliothrips biformis, Dichromothrips corbetti, Dichromothrips ssp., Enneothrips flavens, Frankliniella spp. such as Frankliniella fusca, Frankliniella occidentalis, Frankliniella tritici; Heliothrips spp., Hercinothrips femoralis, Kakothrips spp., Rhipiphorothrips cruentatus, Scirtothrips spp. such as Scirtothrips citri; Taeniothrips cardamoni, Thrips spp. such as Thrips oryzae, Thrips palmi, Thrips tabaci;

termites (Isoptera), e.g. Calotermes flavicollis, Coptotermes formosanus, Heterotermes aureus, Heterotermes longiceps, Heterotermes tenuis, Leucotermes flavipes, Odontotermes spp., Reticulitermes spp. such as Reticulitermes speratus, Reticulitermes flavipes, Reticulitermes grassei, Reticulitermes lucifugus, Reticulitermes santonensis, Reticulitermes virginicus; Termes natalensis,

cockroaches (Blattaria-Blattodea), e.g. Acheta domesticus, Blatta orientalis, Blattella asahinae, Blattella germanica, Gryllotalpa spp., Leucophaea maderae, Locusta spp., Melanoplus spp., Periplaneta americana, Periplaneta australasiae, Periplaneta brunnea, Periplaneta fuligginosa, Periplaneta japonica,

bugs, aphids, leafhoppers, whiteflies, scale insects, cicadas (Hemiptera), e.g. Acrosternum spp. such as Acrosternum hilare; Acyrthosipon spp. such as Acyrthosiphon onobrychis, Acyrthosiphon pisum; Adelges laricis, Aeneolamia spp., Agonoscena spp., Aleurodes spp., Aleurolobus barodensis, Aleurothrixus spp., Amrasca spp., Anasa tristis, Antestiopsis spp., Anuraphis cardui, Aonidiella spp., Aphanostigma piri, Aphidula nasturtii, Aphis spp. such as Aphis fabae, Aphis forbesi, Aphis gossypii, Aphis grossulariae, Aphis pomi, Aphis sambuci, Aphis schneideri, Aphis spiraecola; Arboridia apicalis, Arilus critatus, Aspidiella spp., Aspidiotus spp., Atanus spp., Aulacorthum solani, Bemisia spp. such as Bemisia argentifolii, Bemisia tabaci; Blissus spp. such as Blissus leucopterus; Brachycaudus cardui, Brachycaudus helichrysi, Brachycaudus persicae, Brachycaudus prunicola, Brachycolus spp., Brevicoryne brassicae, Calligypona marginata, Calocoris spp., Campylomma livida, Capitophorus horni, Carneocephala fulgida, Cavelerius spp., Ceraplastes spp., Ceratovacuna lanigera, Cercopidae, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as Cimex hemipterus, Cimex lectularius; Coccomytilus halli, Coccus spp., Creontiades dilutus, Cryptomyzus ribis, Cryptomyzus ribis, Cyrtopeltis notatus, Dalbulus spp., Dasynus piperis, Dialeurades spp., Diaphorina spp., Diaspis spp., Dichelops furcatus, Diconocoris hewetti, Doralis spp., Dreyfusia nordmannianae, Dreyfusia piceae, Drosicha spp., Dysaphis spp. such as Dysaphis plantaginea, Dysaphis pyri, Dysaphis radicola; Dysaulacorthum pseudosolani, Dysdercus spp. such as Dysdercus cingulatus, Dysdercus intermedius; Dysmicoccus spp., Empoasca spp. such as Empoasca fabae, Empoasca solana; Eriosoma spp., Erythroneura spp., Eurygaster spp. such as Eurygaster integriceps; Euscelis bilobatus, Euschistus spp. such as Euschistuos heros, Euschistus impictiventris, Euschistus servus; Geococcus coffeae, Halyomorpha spp. such as Halyomorpha halys; Heliopeltis spp., Homalodisca coagulata, Horcias nobilellus, Hyalopterus pruni, Hyperomyzus lactucae, Icerya spp., Idiocerus spp., Idioscopus spp., Laodelphax striatellus, Lecanium spp., Lepidosaphes spp., Leptocorisa spp., Leptoglossus phyllopus, Lipaphis erysimi, Lygus spp. such as Lygus hesperus, Lygus lineolaris, Lygus pratensis; Macropes excavatus, Macrosiphum spp. such as Macrosiphum rosae, Macrosiphum avenae, Macrosiphum euphorbiae; Mahanarva fimbriolata, Megacopta cribraria, Megoura viciae, Melanaphis pyrarius, Melanaphis sacchari, Metcafiella spp., Metopolophium dirhodum, Miridae spp., Monellia costalis, Monelliopsis pecanis, Myzus spp. such as Myzus ascalonicus, Myzus cerasi, Myzus persicae, Myzus varians; Nasonovia ribis-nigri, Nephotettix spp. such as Nephotettix malayanus, Nephotettix nigropictus, Nephotettix parvus, Nephotettix virescens; Nezara spp. such as Nezara viridula; Nilaparvata lugens, Oebalus spp., Oncometopia spp., Orthezia praelonga, Parabemisia myricae, Paratrioza spp., Parlatoria spp., Pemphigus spp. such as Pemphigus bursarius; Pentomidae, Peregrinus maidis, Perkinsiella saccharicida, Phenacoccus spp., Phloeomyzus passerinii, Phorodon humuli, Phylloxera spp., Piesma quadrata, Piezodorus spp. such as Piezodorus guildinii, Pinnaspis aspidistrae, Planococcus spp., Protopulvinaria pyriformis, Psallus seriatus, Pseudacysta persea, Pseudaulacaspis pentagona, Pseudococcus spp. such as Pseudococcus comstocki; Psylla spp. such as Psylla mali, Psylla piri; Pteromalus spp., Pyrilla spp., Quadraspidiotus spp., Quesada gigas, Rastrococcus spp., Reduvius senilis, Rhodnius spp., Rhopalomyzus ascalonicus, Rhopalosiphum spp. such as Rhopalosiphum pseudobrassicas, Rhopalosiphum insertum, Rhopalosiphum maidis, Rhopalosiphum padi; Sagatodes spp., Sahlbergella singularis, Saissetia spp., Sappaphis mala, Sappaphis mali, Scaphoides titanus, Schizaphis graminum, Schizoneura lanuginosa, Scotinophora spp., Selenaspidus articulatus, Sitobion avenae, Sogata spp., Sogatella furcifera, Solubea insularis , Stephanitis nashi, Stictocephala festina, Tenalaphara malayensis, Thyanta spp. such as Thyanta perditor; Tibraca spp., Tinocallis caryaefoliae, Tomaspis spp., Toxoptera spp. such as Toxoptera aurantii; Trialeurodes spp. such as Trialeurodes vaporariorum; Triatoma spp., Trioza spp., Typhlocyba spp., Unaspis spp. such as Unaspis yanonensis; and Viteus vitifolii,

ants, bees, wasps, sawflies (Hymenoptera), e.g. Athalia rosae, Atta capiguara, Atta cephalotes, Atta cephalotes, Atta laevigata, Atta robusta, Atta sexdens, Atta texana, Bombus spp., Camponotus floridanus, Crematogaster spp., Dasymutilla occidentalis, Diprion spp., Dolichovespula maculata, Hoplocampa spp. such as Hoplocampa minuta, Hoplocampa testudinea; Lasius spp. such as Lasius niger, Linepithema humile, Monomorium pharaonis, Paravespula germanica, Paravespula pennsylvanica, Paravespula vulgaris, Pheidole megacephala, Pogonomyrmex barbatus, Pogonomyrmex californicus, Polistes rubiginosa, Solenopsis geminata, Solenopsis invicta, Solenopsis richteri, Solenopsis xyloni, Vespa spp. such as Vespa crabro, and Vespula squamosa,

crickets, grasshoppers, locusts (Orthoptera), e.g. Acheta domestica, Calliptamus italicus, Chortoicetes terminifera, Dociostaurus maroccanus, Gryllotalpa africana, Gryllotalpa gryllotalpa, Hieroglyphus daganensis, Kraussaria angulifera, Locusta migratoria, Locustana pardalina, Melanoplus bivittatus, Melanoplus femurrubrum, Melanoplus mexicanus, Melanoplus sanguinipes, Melanoplus spretus, Nomadacris septemfasciata, Oedaleus senegalensis, Schistocerca americana, Schistocerca gregaria, Tachycines asynamorus, and Zonozerus variegatus,

arachnids (Arachnida), such as acari, e.g. of the families Argasidae, Ixodidae and Sarcoptidae, such as Amblyomma spp. (e.g. Amblyomma americanum, Amblyomma variegatum, Amblyomma maculatum), Argas spp. (e.g. Argas persicus), Boophilus spp. (e.g. Boophilus annulatus, Boophilus decoloratus, Boophilus microplus), Dermacentor silvarum, Dermacentor andersoni, Dermacentor variabilis, Hyalomma spp. (e.g. Hyalomma truncatum), Ixodes spp. (e.g. Ixodes ricinus, Ixodes rubicundus, Ixodes scapularis, Ixodes holocyclus, Ixodes pacificus), Ornithodorus spp. (e.g. Ornithodorus moubata, Ornithodorus hermsi, Ornithodorus turicata), Ornithonyssus bacoti, Otobius megnini, Dermanyssus gallinae, Psoroptes spp. (e.g. Psoroptes ovis), Rhipicephalus spp. (e.g. Rhipicephalus sanguineus, Rhipicephalus appendiculatus, Rhipicephalus evertsi), Rhizoglyphus spp., Sarcoptes spp. (e.g. Sarcoptes scabiei), and Eriophyidae spp. such as Acaria sheldoni, Aculops spp. (e.g. Aculops pelekassi) Aculus spp. (e.g. Aculus schlechtendali), Epitrimerus pyri, Phyllocoptruta oleivora and Eriophyes spp. (e.g. Eriophyes sheldoni); Tarsonemidae spp. such as Hemitarsonemus spp., Phytonemus pallidus and Polyphagotarsonemus latus, Stenotarsonemus spp.; Tenuipalpidae spp. such as Brevipalpus spp. (e.g. Brevipalpus phoenicis); Tetranychidae spp. such as Eotetranychus spp., Eutetranychus spp., Oligonychus spp., Tetranychus cinnabarinus, Tetranychus kanzawai, Tetranychus pacificus, Tetranychus telarius and Tetranychus urticae; Bryobia praetiosa, Panonychus spp. (e.g. Panonychus ulmi, Panonychus citri), Metatetranychus spp. and Oligonychus spp. (e.g. Oligonychus pratensis), Vasates lycopersici; Araneida, e.g. Latrodectus mactans, and Loxosceles reclusa. And Acarus siro, Chorioptes spp., Scorpio maurus

fleas (Siphonaptera), e.g. Ceratophyllus spp., Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Geophilus spp., Scutigera spp. such as Scutigera coleoptrata;

millipedes (Diplopoda), e.g. Blaniulus guttulatus, Narceus spp.,

Earwigs (Dermaptera), e.g. forficula auricularia,

lice (Phthiraptera), e.g. Damalinia spp., Pediculus spp. such as Pediculus humanus capitis, Pediculus humanus corporis; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp.,

springtails (Collembola), e.g. Onychiurus ssp. such as Onychiurus armatus,

They are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species such as Aphelenchoides besseyi; Sting nematodes, Belonolaimus longicaudatus and other Belonolaimus species; Pine nematodes, Bursaphelenchus lignicolus Mamiya et Kiyohara, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus brachyurus, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species such as Tylenchulus semipenetrans; Dagger nematodes, Xiphinema species; and other plant parasitic nematode species.

Examples of further pest species which may be controlled by compounds of formula (I) include: from the class of the Bivalva, for example, Dreissena spp.; from the class of the Gastropoda, for example, Anion spp., Biomphalaria spp., Bulinus spp., Deroceras spp., Galba spp., Lymnaea spp., Oncomelania spp., Succinea spp.; from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti; from the order of the Isopoda, for example, Armadillidium vulgare, Oniscus asellus, Porcellio scaber; from the order of the Symphyla, for example, Scutigerella immaculata.

Further examples of pest species which may be controlled by compounds of formula (I) include: Anisoplia austriaca, Apamea spp., Austroasca viridigrisea, Baliothrips biformis, Caenorhabditis elegans, Cephus spp., Ceutorhynchus napi, Chaetocnema aridula, Chilo auricilius, Chilo indicus, Chilo polychrysus, Chortiocetes terminifera, Cnaphalocroci medinalis, Cnaphalocrosis spp., Colias eurytheme, Collops spp., Cornitermes cumulans, Creontiades spp., Cyclocephala spp., Dalbulus maidis, Deraceras reticulatum, Diatrea saccharalis, Dichelops furcatus, Dicladispa armigera, Diloboderus spp. such as Diloboderus abderus; Edessa spp., Epinotia spp., Formicidae, Geocoris spp., Globitermes sulfureus, Gryllotalpidae, Halotydeus destructor, Hipnodes bicolor, Hydrellia philippina, Julus spp., Laodelphax spp., Leptocorsia acuta, Leptocorsia oratorius, Liogenys fuscus, Lucillia spp., Lyogenys fuscus, Mahanarva spp., Maladera matrida, Marasmia spp., Mastotermes spp., Mealybugs, Megascelis ssp, Metamasius hemipterus, Microtheca spp., Mocis latipes, Murgantia spp., Mythemina separata, Neocapritermes opacus, Neocapritermes parvus, Neomegalotomus spp., Neotermes spp., Nymphula depunctalis, Oebalus pugnax, Orseolia spp. such as Orseolia oryzae; Oxycaraenus hyalinipennis, Plusia spp., Pomacea canaliculata, Procornitermes ssp, Procornitermes triacifer, Psylloides spp., Rachiplusia spp., Rhodopholus spp., Scaptocoris castanea, Scaptocoris spp., Scirpophaga spp. such as Scirpophaga incertulas, Scirpophaga innotata; Scotinophara spp. such as Scotinophara coarctata; Sesamia spp. such as Sesamia inferens, Sogaella frucifera, Solenapsis geminata, Spissistilus spp., Stalk borer, Stenchaetothrips biformis, Steneotarsonemus spinki, Sylepta derogata, Telehin licus, Trichostrongylus spp.

In a preferred embodiment of the invention, in the methods and uses according to the invention, the compounds of the present invention, including their stereoisomers, salts, tautomers and N-oxides, are particularly useful for controlling chewing-biting pests, in particular insects from the order of Lepidoptera and Coleoptera.

In a further preferred embodiment of the invention, in the methods and uses according to the invention, the compounds of the present invention, including their stereoisomers, salts, tautomers and N-oxides, are particularly useful for controlling sucking or piercing insects, in particular insects from the order Diptera.

Therefore, in an especially preferred embodiment of the invention, in the methods and uses according to the invention, the ryanodine-modulator insecticide resistant insect is from the order Lepidoptera, Coleoptera or Diptera.

In one embodiment of the invention, the ryanodine-modulator insecticide resistant insect is from the order Lepidoptera and is selected from Agrotis ypsllon, Agrotis segetum, Alabama argillacea, Antibarsia gemmatalis, Argyresthia conjugella, Autographa gamma, Bupalus piniarius, Cacoecia munnana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecllia ambiguella, Evetna bouliana, Feltia subterranea, Galleria mellonella, Grapholitha funebrana, Grapholitha molesta, Heliothis armigera, Heliothi:s virescens, Heliothis zea, Hellula undalis, Hibernia defoliaria, Hyphantna cunea, Hyponomeuta malinellus, Keifena lycopersicella, Lambdina fiscellaria, Laphygma exigua, Leucoptera coffeella, Leucoptera scitella, Lithocolletis blancardella, Lobesia botrana, Loxostege sticticalis, Lymantna dispar, Lymantna monacha, Lyonetia clerkella, Malacosoma neustna, Mamestra brassicae, Orgyia pseudotsugata, Ostrinia nubilalis, Panolis flammea, Pectinophora gossypiella, Pendroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix vindana, Trichoplusia ni and Zeiraphera canadensis.

In another embodiment of the invention, the ryanodine-modulator insecticide resistant insect is from the order Coleoptera (beetles) and is selected from Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophllus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popilla japonica, Sitona lineatus and Sitophilus granaria. In a preferred embodiment of the invention, the ryanodine-modulator insecticide resistant insect is a potato beetle, more specifically the Colorado potato beetle.

In another embodiment of the invention, the ryanodine-modulator insecticide resistant insect is from the order Diptera and is selected from Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellana, Chrysops discalis, Chrysops silacea, Chrysops atlanticus, Cochliompa hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophllus intestinalis, Glossina morsitans, Glossina palpalis, Glossina fuscipes, Glossina tachinoides, Haematobia irritans, Haplodiplosi:s equestris, Hippelates spp., Hylemyia platura, Hypoderma lineata, Leptoconops torrens, Liriomyza sativae, Liriomyza trifolii, Lucilia caprin, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis ceras, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa.

In a further preferred embodiment of the invention, in the methods and uses according to the invention, the ryanodine-modulator insecticide resistant insect is selected from thrips, hoppers and whitefly.

In particular, in the methods and uses according to the invention, the ryanodine-modulator insecticide resistant insect is one or more of Tuta absoluta, Pieris rapae, Trichoplusia ni, Plutella xylostella, Spodoptera littoralis, Spodoptera frugiperda, Crocidolomia pavonana, Cnaphalocerus medinalis, Sesamia inferens, Chilo suppressalis, Pyrausta furnacalis, Thermesia gemmatalis, Liriomyza sp., Leptinotarsus decemlineata, Epitrix sp., Phyllotreta cruciferae, Meligethes aeneus, Hypera brunneipennis;

Nilaparvata lugens, Nephotettix virens;

Acyrthosiphum pisum, Bemisia tabaci, Bemisia argentifolii, Euschisuts heros, Nezara viridula, Piezodurus guildingi, Lygus hesperus, thrips spp., Frankliniella occidentalis;

Agriothes sp., Agrotis ypsilon, Phyllotreta spp.

Cydia pomonella, Elasmopalpus lignosellus, Heliothis armigera, Heliothis virescens, Heliothis zea;

Agriotes lineatus, Agriotes obscurus, Anthonomus grandis, Diabrotica longicornis, Diabrotica semipunctata, Diabrotica 12-punctata, Diabrotica speciosa, Diabrotica virgifera, Epitrix hirtipennis, Phaedon cochlearieae, Phyllotreta striolata.

In particular, in the methods and uses according to the invention, the ryanodine-modulator insecticide resistant insect is one or more of Tuta absoluta, Pieris rapae, Trichoplusia ni, Plutella xylostella, Chilo suppressalis, Liriomyza sp., Leptinotarsus decemlineata, Epitrix sp., Phyllotreta cruciferae, Franklinella occidentalis, Bemisia tabaci, Bemisia argentifolii, Agrotis ypsilon.

In particular, in the methods and uses according to the invention, the ryanodine-modulator insecticide resistant insect is one or more of Agrotis ypsilon, Heliothis virescens, Plutella xylostella, Agriotes lineatus, Diabrotica virgifera, Hypera brunneipennis, Leptinotarsus decemlineata, Phyllotreta striolata.

In another embodiment, in the methods and uses according to the invention, the following application types are used:

TABLE AP-T

| Appl. | | |
|---|---|---|
| type | Crop | Pest |
| AP-T-1 | Soybeans | Spodoptera littoralis |
| AP-T-2 | Soybeans | Anticarsia gemmatalis |
| AP-T-3 | Soybeans | Spodoptera exigua |
| AP-T-4 | Soybeans | Stinkbug |
| AP-T-5 | Soybeans | Helicoverpa sp. |
| AP-T-6 | Soybeans | Spodoptera eridania |
| AP-T-7 | Corn | Spodoptera Frugiperta |
| AP-T-8 | Corn | Spodoptera exigua |
| AP-T-9 | Rice | Sesamia inferens |
| AP-T-10 | Rice | Cnaphalocerus medinalis |
| AP-T-11 | Rice | Chilo suppressalis |
| AP-T-12 | Rice | Leptocorisa oratorius |
| AP-T-13 | Rice | Brown plant hopper |
| AP-T-14 | Cotton | Spodoptera littoralis |
| AP-T-15 | Cotton | Thrips spp. |
| AP-T-16 | Cotton | Spodoptera eridania |
| AP-T-17 | Cotton | Helicoverpa sp. |
| AP-T-18 | Canola | Pollen beetle |
| AP-T-19 | SPC | Tuta Absoluta |
| AP-T-20 | SPC | Fruit Borer |
| AP-T-21 | SPC | Spodoptera littoralis |
| AP-T-22 | SPC | Plusia gamma |
| AP-T-23 | SPC | Plutella xylostella |
| AP-T-24 | SPC | Frankliniella occidentalis |
| AP-T-25 | SPC | Trichoplusia ni |
| AP-T-26 | SPC | Pieris rapae |
| AP-T-27 | SPC | Spodoptera sp. |
| AP-T-28 | SPC | Crocidolomia pavonana |
| AP-T-29 | SPC | Pyrausta furnacalis |
| AP-T-30 | SPC | Liromyza trifolii |
| AP-T-31 | SPC | Cydia pomonella |
| AP-T-32 | SPC | Epitrix sp. |
| AP-T-33 | SPC | Leptinotarsa decemlineata |
| AP-T-34 | SPC | Bemisia tabaci |
| AP-T-35 | SPC | Thrips tabaci |
| AP-T-36 | SPC | Spodoptera eridania |
| AP-T-37 | SPC | Lobesia botrana |
| AP-T-38 | SPC | Altica chapybea |
| AP-T-39 | SPC | Phyllocnistis citrella |
| AP-T-40 | Onions | Pyrausta furnacalis |
| AP-T-41 | Onions | Liromyza trifolii |
| AP-T-42 | Onions | Cydia pomonella |
| AP-T-43 | Onions | Epitrix sp. |
| AP-T-44 | Onions | Leptinotarsa decemlineata |
| AP-T-45 | Onions | Bemisia tabaci |
| AP-T-46 | Onions | Thrips tabaci |
| AP-T-47 | Onions | Spodoptera eridania |
| AP-T-48 | Onions | Lobesia botrana |
| AP-T-49 | Onions | Altica chapybea |
| AP-T-50 | Onions | Phyllocnistis citrella |
| AP-T-51 | ST | Agrotis ipsilon |
| AP-T-52 | ST | Spodoptera frugiperta |
| AP-T-53 | ST | Phyllotreta sp. |
| AP-T-54 | ST | Stem Girdler |
| AP-T-55 | ST | Agriotes sp. |
| AP-T-56 | ST | Delia platura |

(Abbreviations: SPC = specialty crops; ST = seed treatment)

Formulations

In the methods and uses according to the invention, the compounds of formula (I) are used in the form of agrochemical compositions comprising an auxiliary and at least one compound of formula (I) according to the invention, or a stereoisomer, salt, tautomer or N-oxide thereof.

An agrochemical composition comprises a pesticidally effective amount of a compound of formula (I). The term “effective amount” denotes an amount of the composition or of the compounds I, which is sufficient for controlling invertebrate pests on cultivated plants or in the protection of materials and which does not result in a substantial damage to the treated plants or material. Such an amount can vary in a broad range and is dependent on various factors, such as the invertebrate (e.g. insect) species to be controlled, the treated cultivated plant or material, the climatic conditions and the specific compound I used.

The compounds I, and their stereoisomers, salts, tautomers and N-oxides, can be converted into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005. Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide.

Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

ii) Dispersible Concentrates (DC)

5-25 wt % of a compound I according to the invention and 1-10 wt % dispersant (e. g. polyvinylpyrrolidone) are dissolved in up to 100 wt % organic solvent (e.g. cyclohexanone). Dilution with water gives a dispersion.

iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

vi) Water-Dispersible Granules and Water-Soluble Granules (WG, SG)

50-80 wt % of a compound I according to the invention are ground finely with addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance.

ix) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

x) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsules. The monomers amount to 1-10 wt %. The wt % relate to the total CS composition.

xi) Dustable Powders (DP, DS)

1-10 wt % of a compound I according to the invention are ground finely and mixed intimately with up to 100 wt % solid carrier, e.g. finely divided kaolin.

xii) Granules (GR, FG)

0.5-30 wt % of a compound I according to the invention is ground finely and associated with up to 100 wt % solid carrier (e.g. silicate). Granulation is achieved by extrusion, spray-drying or the fluidized bed.

xiii) Ultra-Low Volume Liquids (UL)

1-50 wt % of a compound I according to the invention are dissolved in up to 100 wt % organic solvent, e.g. aromatic hydrocarbon.

The compositions types i) to xiii) may optionally comprise further auxiliaries, such as 0.1-1 wt % bactericides, 5-15 wt % anti-freezing agents, 0.1-1 wt % anti-foaming agents, and 0,1-1 wt % colorants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100% (according to NMR spectrum).

In one embodiment, a suspoconcentration (SC) is preferred for the agrochemical application. In one sub-embodiment thereof, the SC agrochemical composition comprises between 50 to 500 g/L (grams per Litre), or between 100 and 250 g/L, or 100 g/L or 150 g/L or 200 g/L or 250 g/L. In a further embodiment, thegranules according to formulation type xii are especially preferred for the application in rice.

Water-soluble concentrates (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.001 to 1 kg per ha, more preferably from 0.005 to 0.9 kg per ha, in particular from 0.005 to 0.5 kg per ha.

In treatment of plant propagation materials such as seeds, e. g. by dusting, coating or drenching seed, amounts of active substance of from 0.1 to 1000 g, preferably from 0.1 to 300 g, more preferably from 0.1 to 100 g and most preferably from 0.25 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and other pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds I and/or active substances, e.g. from the groups M or F, may be mixed by the user in a spray tank and further auxiliaries and additives may be added, if appropriate.

In a further embodiment, either individual components of the composition according to the invention or partially premixed components, e. g. components comprising compounds I and/or active substances from the group M or F, can be applied jointly (e.g. after tank mix) or consecutively.

Mixtures

In the methods and uses according to the invention, the compounds of the present invention, including their stereoisomers, salts, tautomers and N-oxides, may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

In a particular embodiment of the invention, in the methods and uses according to the invention, the compound of formula I is combined with one or more other pesticidally active compound(s) II selected from insecticides or fungicides.

Therefore, the present invention also relates to methods and uses, wherein a mixture or composition comprising at least one compound of formula (I), or a stereoisomer, tautomer, N-oxide or agriculturally or veterinarily acceptable salt thereof, and at least one further pesticide.

The compounds of formula (I), and their stereoisomers, salts, tautomers and N-oxides, may be applied with other insecticides as compound II, which are either listed as Ryanodine-modulator Insecticides above (which may be useful apart from the mentioned resistant insects), or are listed in the following categorized list M of pesticides, which are, whenever possible, classified according to the Insecticide Resistance Action Committee (IRAC):

M.1 Acetylcholine esterase (AChE) inhibitors from the class of

M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of

M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O— (methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;

M.2. GABA-gated chloride channel antagonists such as:

M.2A cyclodiene organochlorine compounds, as for example endosulfan or chlordane; or

M.2B fiproles (phenylpyrazoles), as for example ethiprole, fipronil, flufiprole, pyrafluprole and pyriprole;

M.3 Sodium channel modulators from the class of

M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin,metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or

M.3B sodium channel modulators such as DDT or methoxychlor;

M.4 Nicotinic acetylcholine receptor agonists (nAChR) from the class of

M.4A neonicotinoids, for example acteamiprid, chlothianidin, dinotefuran, imidacloprid, nitenpyram, thiacloprid and thiamethoxam; or or the compounds

M.4A.1: 1-[(6-chloro-3-pyridinyl)methyl]-2,3,5,6,7,8-hexahydro-9-nitro-(5S,8R)-5,8-Epoxy-1H-imidazo[1,2-a]azepine; or

M.4A.2: 1-[(6-chloro-3-pyridyl)methyl]-2-nitro-1-[(E)-pentylideneamino]guanidine; or

M.4A.3: 1-[(6-chloro-3-pyridyl)methyl]-7-methyl-8-nitro-5-propoxy-3,5,6,7-tetrahydro-2H-imidazo[1,2-a]pyridine; or

M.4B nicotine.

M.5 Nicotinic acetylcholine receptor allosteric activators from the class of spinosyns, for example spinosad or spinetoram;

M.6 Chloride channel activators from the class of avermectins and milbemycins, for example abamectin, emamectin benzoate, ivermectin, lepimectin or milbemectin;

M.7 Juvenile hormone mimics, such as

M.7A juvenile hormone analogues as hydroprene, kinoprene and methoprene; or others as

M.7B fenoxycarb, or

M.7C pyriproxyfen;

M.8 miscellaneous non-specific (multi-site) inhibitors, for example

M.8A alkyl halides as methyl bromide and other alkyl halides, or

M.8B chloropicrin, or

M.8C sulfuryl fluoride, or

M.8D borax, or

M.8E tartar emetic;

M.9 Selective homopteran feeding blockers, for example

M.9B pymetrozine, or

M.9C flonicamid;

M.10 Mite growth inhibitors, for example

M.10A clofentezine, hexythiazox and diflovidazin, or

M.10B etoxazole;

M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;

M.12 Inhibitors of mitochondrial ATP synthase, for example

M.12A diafenthiuron, or

M.12B organotin miticides such as azocyclotin, cyhexatin or fenbutatin oxide, or M.12C propargite, or

M.12D tetradifon;

M.13 Uncouplers of oxidative phosphorylation via disruption of the proton gradient, for example chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disruptors, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example

M.20A hydramethylnon, or

M.20B acequinocyl, or

M.20C fluacrypyrim;

M.21 Mitochondrial complex I electron transport inhibitors, for example

M.21A METI acaricides and insecticides such as fenazaquin, fenpyroximate, pyrimidifen, pyridaben, tebufenpyrad or tolfenpyrad, or

M.21B rotenone;

M.22 Voltage-dependent sodium channel blockers, for example

M.22A indoxacarb, or

M.22B metaflumizone; or

M.22C 1-[(E)-[2-(4-cyanophenyl)-1-[3-(trifluoromethyl)phenyl]ethylidene]amino]-3-[4-(difluoromethoxy)phenyl]urea;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example

M.24A phosphine such as aluminium phosphide, calcium phosphide, phosphine or zinc phosphide, or

M.24B cyanide.

M.25 Mitochondrial complex II electron transport inhibitors, such as beta-ketonitrile derivatives, for example cyenopyrafen or cyflumetofen;

M.X insecticidal active compounds of unknown or uncertain mode of action, as for example afidopyropen, azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, pyflubumide or the compounds

M.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound

M.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound

M.X.3: 11-(4-chloro-2,6-dimethylphenyl)-12-hydroxy-1,4-dioxa-9-azadispiro[4.2.4.2]-tetradec-11-en-10-one, or the compound

M.X.4: 3-(4′-fluoro-2,4-dimethylbiphenyl-3-yl)-4-hydroxy-8-oxa-1-azaspiro[4.5]dec-3-en-2-one, or the compound

M.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, 1-1582), or

M.X.6: a compound selected from the group of

M.X.6a: (E/Z)-N-[1-[(6-chloro-3-pyridyl)methyl]-2-pyridylidene]-2,2,2-trifluoro-acetamide;

M.X.6b: (E/Z)-N-[1-[(6-chloro-5-fluoro-3-pyridyl)methyl]-2-pyridylidene]-2,2,2-trifluoro-acetamide;

M.X.6c: (E/Z)-2,2,2-trifluoro-N-[1-[(6-fluoro-3-pyridyl)methyl]-2-pyridylidene]acetamide;

M.X.6d: (E/Z)-N-[1-[(6-bromo-3-pyridyl)methyl]-2-pyridylidene]-2,2,2-trifluoro-acetamide;

M.X.6e: (E/Z)-N-[1-[1-(6-chloro-3-pyridyl)ethyl]-2-pyridylidene]-2,2,2-trifluoro-acetamide;

M.X.6f: (E/Z)-N-[1-[(6-chloro-3-pyridyl)methyl]-2-pyridylidene]-2,2-difluoro-acetamide;

M.X.6g: (E/Z)-2-chloro-N-[1-[(6-chloro-3-pyridyl)methyl]-2-pyridylidene]-2,2-difluoro-acetamide;

M.X.6h: (E/Z)-N-[1-[(2-chloropyrimidin-5-yl)methyl]-2-pyridylidene]-2,2,2-trifluoro-acetamide and

M.X.6i: (E/Z)-N-[1-[(6-chloro-3-pyridyl)methyl]-2-pyridylidene]-2,2,3,3,3-pentafluoro-propanamide); or

M.X.7: triflumezopyrim; or

M.X.8: 4-[5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide, or

M.X.9: 3-[3-chloro-5-(trifluoromethyl)phenyl]-4-oxo-1-(pyrimidin-5-ylmethyl)pyrido[1,2-a]pyrimidin-1-ium-2-olate; or

M.X.10: 8-chloro-N-[2-chloro-5-methoxyphenyl)sulfonyl]-6-trifluoromethyl)-imidazo[1,2-a]pyridine-2-carboxamide; or

M.X.11: 4-[5-(3,5-dichlorophenyl)-5-(trifluoromethyl)-4H-isoxazol-3-yl]-2-methyl-N-(1-oxothietan-3-yl)benzamide; or

M.X.12: 5-[3-[2,6-dichloro-4-(3,3-dichloroallyloxy)phenoxy]propoxy]-1H-pyrazole; or

M.Y Biopesticides, e.g.

M.Y-1: Microbial pesticides with insecticidal, acaricidal, molluscidal and/or nematicidal activity: Bacillus firmus, B. thuringiensis ssp. israelensis, B. t. ssp. galleriae, B. t. ssp. kurstaki, Beauveria bassiana, Burkholderia sp., Chromobacterium subtsugae, Cydia pomonella granulosis virus, Isaria fumosorosea, Lecanicillium longisporum, L. muscarium (formerly Verticillium lecanii), Metarhizium anisopliae, M. anisopliae var. acridum, Paecilomyces fumosoroseus, P. lilacinus, Paenibacillus poppiliae, Pasteuria spp., P. nishizawae, P. reneformis, P. usagae, Pseudomonas fluorescens, Steinernema feltiae, Streptomces galbus;

M.Y-2) Biochemical pesticides with insecticidal, acaricidal, molluscidal, pheromone and/or nematicidal activity: L-carvone, citral, (E,Z)-7,9-dodecadien-1-ylacetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-ylacetate, Z-7-tetradecen-2-one, Z-9-tetradecen-1-ylacetate, Z-11-tetradecenal, Z-11-tetradecen-1-ol, Acacia negra extract, extract of grapefruit seeds and pulp, extract of Chenopodium ambrosiodae, Catnip oil, neem oil, Quillay extract, Tagetes oil.

The commercially available compounds of the group M listed above may be found in The Pesticide Manual, 15th Edition, C. D. S. Tomlin, British Crop Protection Council (2011) among other publications.

The quinoline derivative flometoquin is shown in WO2006/013896. The aminofuranone compounds flupyradifurone is known from WO 2007/115644. The sulfoximine compound sulfoxaflor is known from WO2007/149134. The pyrethroid momfluorothrin is known from U.S. Pat. No. 6,908,945. The pyrazole acaricide pyflubumide is known from WO2007/020986. The isoxazoline compound M.X.1 has been described in WO2005/085216, M.X.8 in WO2009/002809 and in WO2011/149749 and the isoxazoline M.X.9 in WO2013/050317. The pyripyropene derivative M.X.2 has been described in WO 2006/129714. The spiroketal-substituted cyclic ketoenol derivative M.X.3 is known from WO2006/089633 and the biphenyl-substituted spirocyclic ketoenol derivative M.X.4 from WO2008/067911. Triazoylphenylsulfide like M.X.5 have been described in WO2006/043635 and biological control agents on basis of bacillus firmus in WO2009/124707. The neonicotionids M4A.1 is known from WO20120/069266 and WO2011/06946, the M.4A.2 from WO2013/003977, the M4A.3.from WO2010/069266. The metaflumizone analogue M.22C is described in CN 10171577.

The compounds M.X.6a) to M.X.6i) listed in M.X.6 have been described in WO2012/029672. The mesoionic antagonist compound M.X.8 was described in WO2012/092115, the nematicide M.X.9 in WO2013/055584 and the Pyridalyl-type analogue M.X.12 in WO2010/060379.

Biopesticides

The biopesticides from group M.Y, and from group F.XIII) as described below, their preparation and their biological activity e.g. against harmful fungi, pests is known (e-Pesticide Manual V 5.2 (ISBN 978 1 901396 85 0) (2008-2011); http://www.epa.gov/opp00001/biopesticides/, see product lists therein; http://www.omri.org/omri-lists, see lists therein; Bio-Pesticides Database BPDB http://sitem.herts.ac.uk/aeru/bpdb/, see A to Z link therein). Many of these biopesticides are registered and/or are commercially available: aluminium silicate (SCREEN™ DUO from Certis LLC, USA), Ampelomyces quisqualis M-10 (e.g. AQ 10C) from Intrachem Bio GmbH & Co. KG, Germany), Ascophyllum nodosum (Norwegian kelp, Brown kelp) extract (e.g. ORKA GOLD from Becker Underwood, South Africa), Aspergillus flavus NRRL 21882 (e.g. AFLA-GUARD® from Syngenta, CH), Aureobasidium pullulans (e.g. BOTECTOR® from bio-ferm GmbH, Germany), Azospirillum brasilense XOH (e.g. AZOS from Xtreme Gardening, USA USA or RTI Reforestation Technologies International; USA), Bacillus amyloliquefaciens IT-45 (CNCM 13800, NCBI 1091041) (e.g. RHIZOCELL C from ITHEC, France), B. amyloliquefaciens subsp. plantarum MBI600 (NRRL B-50595, deposited at United States Department of Agriculture) (e.g. INTEGRAL®, CLARITY, SUBTILEX NG from Becker Underwood, USA), B. pumilus QST 2808 (NRRL Accession No. B 30087) (e.g. SONATA® and BALLAD® Plus from AgraQuest Inc., USA), B. subtilis GB03 (e.g. KODIAK from Gustafson, Inc., USA), B. subtilis GB07 (EPIC from Gustafson, Inc., USA), B. subtilis QST-713 (NRRL-Nr. B 21661 in RHAPSODY®, SERENADE® MAX and SERENADE® ASO from Agra-Quest Inc., USA), B. subtilis var. amylolique [symbol] faciens FZB24 (e.g. TAEGRO® from Novozyme Biologicals, Inc., USA), B. subtilis var. amyloliquefaciens D747 (e.g. Double Nickel 55 from Certis LLC, USA), Bacillus thuringiensis ssp. kurstaki SB4 (e.g. BETA PRO® from Becker Underwood, South Africa), Beauveria bassiana GHA (BOTANIGARD® 22WGP from Laverlam Int. Corp., USA), B. bassiana 12256 (e.g. BIOEXPERT® SC from Live Sytems Technology S.A., Colombia), B. bassiana PRPI 5339 (ARSEF number 5339 in the USDA ARS collection of entomopathogenic fungal cultures) (e.g. BROAD [symbol] BAND® from Becker Underwood, South Africa), Bradyrhizobium sp. (e.g. VAULT® from Becker Underwood, USA), B. japonicum (e.g. VAULT® from Becker Underwood, USA), Candida oleophila I-82 (e.g. ASPIRE® from Ecogen Inc., USA), Candida saitoana (e.g. B10-CURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans CON/M/91-08 (e.g. Contans® WG from Prophyta, Germany), Cryphonectria parasitica (e.g. Endothia parasitica from CNICM, France), Cryptococcus albidus (e.g. YIELD PLUS® from Anchor Bio-Technologies, South Africa), Ecklonia maxima (kelp) extract (e.g. KELPAK SL from Kelp Products Ltd, South Africa), Fusarium oxysporum (e.g. B10-FOX® from S.I.A.P.A., Italy, FUSACLEAN® from Natural Plant Protection, France), Glomus intraradices (e.g. MYC 4000 from ITHEC, France), Glomus intraradices RTI-801 (e.g. MYKOS from Xtreme Gardening, USA or RTI Reforestation Technologies International; USA), grapefruit seeds and pulp extract (e.g. BC-1000 from Chemie S.A., Chile), Isaria fumosorosea Apopka-97 (ATCC 20874) (PFR-97™ from Certis LLC, USA), Lecanicillium muscarium (formerly Verticillium lecanii) (e.g. MYCOTAL from Koppert B V, Netherlands), Lecanicillium longisporum KV42 and KV71 (e.g. VERTALEC® from Koppert BV, Netherlands), Metarhizium anisopliae var. acridum IMI 330189 (deposited in European Culture Collections CABI) (e.g. GREEN MUSCLE® from Becker Underwood, South Africa), M. anisopliae FI-1045 (e.g. BIOCANE® from Becker Underwood Pty Ltd, Australia), M. anisopliae var. acridum FI-985 (e.g. GREEN GUARD® SC from Becker Underwood Pty Ltd, Australia), M. anisopliae F52 (e.g. MET52® Novozymes Biologicals BioAg Group, Canada), M. anisopliae ICIPE 69 (e.g. METATHRI [symbol] POL from ICIPE, Kenya), Metschnikowia fructicola (e.g. SHEMER® from Agrogreen, Israel), Microdochium dimerum (e.g. ANTIBOT® from Agrauxine, France), Neem oil (e.g. TRILOGY®, TRIACT® 70 EC from Certis LLC, USA), Paecilomyces fumosoroseus strain FE 9901 (e.g. NO FLY™ from Natural Industries, Inc., USA), P. lilacinus DSM 15169 (e.g. NEMATA® SC from Live Systems Technology S.A., Colombia), P. lilacinus BCP2 (e.g. PL GOLD from Becker Underwood BioAg SA Ltd, South Africa), mixture of Paenibacillus alvei NAS6G6 and Bacillus pumilis (e.g. BAC-UP from Becker Underwood South Africa), Penicillium bilaiae (e.g. JUMP START® from Novozymes Biologicals BioAg Group, Canada), Phlebiopsis gigantea (e.g. ROTSTOP® from Verdera, Finland), potassium silicate (e.g. Sil-MATRIX™ from Certis LLC, USA), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis extract (e.g. REGALIA® from Marrone BioInnovations, USA), Rhizobium leguminosarum bv. phaseolii (e.g. RHIZO-STICK from Becker Underwood, USA), R. I. trifolii (e.g. DORMAL from Becker Underwood, USA), R. I. bv. viciae (e.g. NODULATOR from Becker Underwood, USA), Sinorhizobium meliloti (e.g. DORMAL ALFALFA from Becker Underwood, USA; NITRAGIN® Gold from Novozymes Biologicals BioAg Group, Canada), Steinernema feltiae (NEMA [symbol] SHIELD® from BioWorks, Inc., USA), Streptomyces lydicus WYEC 108 (e.g. Actinovate® from Natural Industries, Inc., USA, U.S. Pat. No. 5,403,584), S. violaceusniger YCED-9 (e.g. DT-9® from Natural Industries, Inc., USA, U.S. Pat. No. 5,968,503), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. atroviride LC52 (e.g. SENTINEL® from Agrimm Technologies Ltd, NZ), T. fertile JM41 R (e.g. RICHPLUS™ from Becker Underwood Bio Ag SA Ltd, South Africa), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB Bio-Innovation AB, Sweden), T. stromaticum (e.g. TRICO-VAB® from C.E.P.L.A.C., Brazil), T. virens GL-21 (also named Gliocladium virens) (e.g. SOIL-GARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy), Ulocladium oudemansii HRU3 (e.g. BOTRY-ZEN® from Botry-Zen Ltd, NZ), Bacillus amyloliquefaciens AP-136 (NRRL B-50614), B. amyloliquefaciens AP-188 (NRRL B-50615), B. amyloliquefaciens AP-218 (NRRL B-50618), B. amyloliquefaciens AP-219 (NRRL B-50619), B. amyloliquefaciens AP-295 (NRRL B-50620), B. mojavensis AP-209 (No. NRRL B-50616), B. solisalsi AP-217 (NRRL B-50617), B. pumilus strain INR-7 (otherwise referred to as BU-F22 (NRRL B-50153) and BU-F33 (NRRL B-50185)), B. simplex ABU 288 (NRRL B-50340) and B. amyloliquefaciens subsp. plantarum MBI600 (NRRL B-50595) have been mentioned i.a. in US patent appl. 20120149571, WO 2012/079073. Beauveria bassiana DSM 12256 is known from US200020031495. Bradyrhizobium japonicum USDA is known from U.S. Pat. NO. 7,262,151. Sphaerodes mycoparasitica IDAC 301008-01 (IDAC=International Depositary Authority of Canada Collection) is known from WO 2011/022809.

Bacillus amyloliquefaciens subsp. plantarum MBI600 having the accession number NRRL B-50595 is deposited with the United States Department of Agriculture on Nov. 10, 2011 under the strain designation Bacillus subtilis 1430. It has also been deposited at The National Collections of Industrial and Marine Bacteria Ltd. (NCIB), Torry Research Station, P.O. Box 31, 135 Abbey Road, Aberdeen, AB9 8DG, Scotland.under accession number 1237 on Dec. 22, 1986. Bacillus amyloliquefaciens MBI600 is known as plant growth-promoting rice seed treatment from Int. J. Microbiol. Res. ISSN 0975-5276, 3(2) (2011), 120-130 and further described e.g. in US 2012/0149571 A1. This strain MBI600 is commercially available as liquid formulation product Integral® (Becker-Underwood Inc., USA). Recently, the strain MBI 600 has been reclassified as Bacillus amyloliquefaciens subsp. plantarum based on polyphasic testing which combines classical microbiological methods relying on a mixture of traditional tools (such as culture-based methods) and molecular tools (such as genotyping and fatty acids analysis). Thus, Bacillus subtilis MBI600 (or MBI 600 or MBI-600) is identical to Bacillus amyloliquefaciens subsp. plantarum MBI600, formerly Bacillus subtilis MBI600.

Metarhizium anisopliae IM133 is commercially available from Becker Underwood as product Green Guard. M. anisopliae var acridium strain IMI 330189 (NRRL-50758) is commercially available from Becker Underwood as product Green Muscle.

Bacillus subtilis strain FB17 was originally isolated from red beet roots in North America (System Appl. Microbiol 27 (2004) 372-379). This Bacillus subtilis strain promotes plant health (US 2010/0260735 A1; WO 2011/109395 A2). B. subtilis FB17 has also been deposited at American Type Culture Collection (ATCC), Manassas, Va., USA, under accession number PTA-11857 on Apr. 26, 2011. Bacillus subtilis strain FB17 may also be referred to as UD1022 or UD10-22.

According to one embodiment of the inventive mixtures, the at least one biopesticide II is selected from the groups M.Y-1 to M.Y-2:

M.Y-1: Microbial pesticides with insecticidal, acaricidal, molluscidal and/or nematicidal activity: Bacillus firmus St 1582, B. thuringiensis ssp. israelensis SUM-6218, B. t. ssp. galleriae SDS-502, B. t. ssp. kurstaki, Beauveria bassiana GHA, B. bassiana H123, B. bassiana DSM 12256, B. bassiana PRPI 5339, Burkholderia sp. A396, Chromobacterium subtsugae PRAA4-1T, Cydia pomonella granulosis virus isolate V22, Isaria fumosorosea Apopka-97, Lecanicillium longisporum KV42, L. longisporum KV71, L. muscarium (formerly Verticillium lecanii), Metarhizium anisopliae FI-985, M. anisopliae FI-1045, M. anisopliae F52, M. anisopliae ICIPE 69, M. anisopliae var. acridum IMI 330189, Paecilomyces fumosoroseus FE 9901, P. lilacinus DSM 15169, P. lilacinus BCP2, Paenibacillus poppiliae Dutky-1940 (NRRL B-2309=ATCC 14706), P. poppiliae KLN 3, P. poppiliae Dutky 1, Pasteuria spp. Ph3, P. nishizawae PN-1, P. reneformis Pr-3, P. usagae, Pseudomonas fluorescens CL 145A, Steinernema feltiae, Streptomces galbus;

M.Y-2: Biochemical pesticides with insecticidal, acaricidal, molluscidal, pheromone and/or nematicidal activity: L-carvone, citral, (E,Z)-7,9-dodecadien-1-ylacetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, Acacia negra extract, extract of grapefruit seeds and pulp, extract of Chenopodium ambrosiodae, Catnip oil, Neem oil, Quillay extract, Tagetes oil;

According to one embodiment of the inventive mixtures, the at least one biopesticide II is selected from group M.Y-1.

According to one embodiment of the inventive mixtures, the at least one biopesticide II is selected from M.Y-2.

According to one embodiment of the inventive mixtures, the at least one biopesticide II is Bacillus amyloliquefaciens subsp. plantarum MBI600. These mixtures are particularly suitable in soybean.

According to another embodiment of the inventive mixtures, the at least one biopesticide II is B. pumilus strain INR-7 (otherwise referred to as BU-F22 (NRRL B-50153) and BU-F33 (NRRL B-50185; see WO 2012/079073). These mixtures are particularly suitable in soybean and corn.

According to another embodiment of the inventive mixtures, the at least one biopesticide II is Bacillus pumilus, preferably B. pumilis strain INR-7 (otherwise referred to as BU-F22 (NRRL B-50153) and BU-F33 (NRRL B-50185). These mixtures are particularly suitable in soybean and corn.

According to another embodiment of the inventive mixtures, the at least one biopesticide II is Bacillus simplex, preferably B. simplex strain ABU 288 (NRRL B-50340). These mixtures are particularly suitable in soybean and corn.

According to another embodiment of the inventive mixtures, the at least one biopesticide II is selected from Trichoderma asperellum, T. atroviride, T. fertile, T. gamsii, T. harmatum; mixture of T. harzia [symbol] num and T. viride; mixture of T. polysporum and T. harzianum; T. stromaticum, T. virens (also named Gliocladium virens) and T. viride; preferably Trichoderma fertile, in particular T. fertile strain JM41 R. These mixtures are particularly suitable in soybean and corn.

According to another embodiment of the inventive mixtures, the at least one biopesticide II is Sphaerodes mycoparasitica, preferably Sphaerodes mycoparasitica strain IDAC 301008-01 (also referred to as strain SMCD2220-01). These mixtures are particularly suitable in soybean and corn.

According to another embodiment of the inventive mixtures, the at least one biopesticide II is Beauveria bassiana, preferably Beauveria bassiana strain PPRI5339. These mixtures are particularly suitable in soybean and corn.

According to another embodiment of the inventive mixtures, the at least one biopesticide II is Metarhizium anisopliae or M. anisopliae var. acridium, preferably selectged from M anisolpiae strain IM133 and M. anisopliae var. acridium strain IMI 330189. These mixtures are particularly suitable in soybean and corn.

According to another embodiment of the inventive mixtures, Bradyrhizobium sp. (meaning any Bradyrhizobium species and/or strain) as biopesticide II is Bradyrhizobium japonicum (B. japonicum). These mixtures are particularly suitable in soybean. Preferably B. japonicum is not one of the strains TA-11 or 532c. B. japonicum strains were cultivated using media and fermentation techniques known in the art, e.g. in yeast extract-mannitol broth (YEM) at 27° C. for about 5 days.

References for various B. japonicum strains are given e.g. in U.S. Pat. No. 7,262,151 (B. japonicum strains USDA 110 (=IITA 2121, SEMIA 5032, RCR 3427, ARS 1-110, Nitragin 61A89; isolated from Glycine max in Florida in 1959, Serogroup 110; Appl Environ Microbiol 60, 940-94, 1994), USDA 31 (=Nitragin 61A164; isolated from Glycine max in Wisconsin in 1941, USA, Serogroup 31), USDA 76 (plant passage of strain USDA 74 which has been isolated from Glycine max in California, USA, in 1956, Serogroup 76), USDA 121 (isolated from Glycine max in Ohio, USA, in 1965), USDA 3 (isolated from Glycine max in Virginia, USA, in 1914, Serogroup 6) and USDA 136 (=CB 1809, SEMIA 586, Nitragin 61A136, RCR 3407; isolated from Glycine max in Beltsville, Md. in 1961; Appl Environ Microbiol 60, 940-94, 1994). USDA refers to United States Department of Agriculture Culture Collection, Beltsville, Md., USA (see e.g. Beltsville Rhizobium Culture Collection Catalog March 1987 ARS-30). Further suitable B. japonicum strain G49 (INRA, Angers, France) is described in Fernandez-Flouret, D. & Cleyet-Marel, J. C. (1987) C R Acad Agric Fr 73, 163-171), especially for soybean grown in Europe, in particular in France. Further suitable B. japonicum strain TA-11 (TA11 NOD+) (NRRL B-18466) is i.a. described in U.S. Pat. No. 5,021,076; Appl Environ Microbiol (1990) 56, 2399-2403 and commercially available as liquid inoculant for soybean (VAULT® NP, Becker Underwood, USA). Further B. japonicum strains as example for biopesticide II are described in US2012/0252672A. Further suitable and especially in Canada commercially available strain 532c (The Nitragin Company, Milwaukee, Wis., USA, field isolate from Wisconsin; Nitragin strain collection No. 61A152; Can J Plant Sci 70 (1990), 661-666).

Other suitable and commercially available B. japonicum strains (see e.g. Appl Environ Microbiol 2007, 73(8), 2635) are SEMIA 566 (isolated from North American inoculant in 1966 and used in Brazilian commercial inoculants from 1966 to 1978), SEMIA 586 (=CB 1809; originally isolated in Maryland, USA but received from Austrailia in 1966 and used in Brazilian inoculants in 1977), CPAC 15 (=SEMIA 5079; a natural varaiant of SEMIA 566 used in commercial inoculants since 1992) and CPAC 7 (=SEMIA 5080; a natural variant of SEMIA 586 used in commercial inoculants since 1992). These strains are especially suitable for soybean grown in Australia or South America, in particular in Brazil. Some of the abovementioned strains have been re-classified as a novel species Bradyrhizobium elkanii, e.g. strain USDA 76 (Can. J. Microbiol., 1992, 38, 501-505).

Another suitable and commercially available B. japonicum strain is E-109 (variant of strain USDA 138, see e.g. Eur. J. Soil Biol. 45 (2009) 28-35; Biol Fertil Soils (2011) 47:81-89, deposited at Agriculture Collection Laboratory of the Instituto de Microbiologia y Zoologia Agricola (IMYZA), Instituto Nacional de Tecnologi'a Agropecuaria (INTA), Castelar, Argentina). This strain is especially suitable for soybean grown in South America, in particular in Argentina. The present invention also relates to mixtures, wherein the at least one biopesticide II is selected from Bradyrhizobium elkanii and Bradyrhizobium liaoningense (B. elkanii and B. liaoningense), more preferably from B. elkanii. These mixtures are particularly suitable in soybean. B. elkanii and liaoningense were cultivated using media and fermentation techniques known in the art, e.g. in yeast extract-mannitol broth (YEM) at 27° C. for about 5 days.

Suitable and commercially available B. elkanii strains are SEMIA 587 and SEMIA 5019 (=29W) (see e.g. Appl Environ Microbiol 2007, 73(8), 2635) and USDA 3254 and USDA 76 and USDA 94. Further commercially available B. elkanii strains are U-1301 and U-1302 (e.g. product Nitroagin® Optimize from Novozymes Bio As S.A., Brazil or NITRASEC for soybean from LAGE y Cia, Brazil). These strains are especially suitable for soybean grown in Australia or South America, in particular in Brazil.

The present invention also relates to mixtures, wherein the at least one biopesticide II is selected from Bradyrhizobium japonicum (B. japonicum) and further comprisies a compound III, wherein compound III is selected from jasmonic acid or salts or derivatives thereof including cis-jasmone, preferably methyl-jasmonate or cis-jasmone.

The present invention also relates to mixtures, wherein biopesticide II is selected from Bradyrhizobium sp. (Arachis) (B. sp. Arachis) which shall describe the cowpea miscellany cross-inoculation group which includes inter alia indigenous cowpea bradyrhizobia on cowpea (Vigna unguiculata), siratro (Macroptilium atropurpureum), lima bean (Phaseolus lunatus), and peanut (Arachis hypogaea). This mixture comprising as biopesticide II B. sp. Arachis is especially suitable for use in peanut, Cowpea, Mung bean, Moth bean, Dune bean, Rice bean, Snake bean and Creeping vigna, in particular peanut.

Suitable and commercially available B. sp. (Arachis) strain is CB1015 (=IITA 1006, USDA 3446 presumably originally collected in India; from Australian Inoculants Research Group; see e.g. http://www.qaseeds.com.au/inoculant_applic.php; Beltsville Rhizobium Culture Collection Catalog March 1987 USDA-ARS ARS-30). These strains are especially suitable for peanut grown in Australia, North America or South America, in particular in Brazil. Further suitable strain is bradyrhizobium sp. PNLO1 (Becker Underwood; ISO Rep Marita McCreary, QC Manager Padma Somasageran; IDENTIFICATION OF RHIZOBIA SPECIES THAT CAN ESTABLISH NITROGEN-FIXING NODULES IN CROTALARIA LONGIROSTRATA. Apr. 29, 2010, University of Massachusetts Amherst: http://www.wpi.edu/Pubs/E-project/Available/E-project-042810-163614/unrestricted/Bisson.Mason._Identification_of_{—}Rhizobia_Species_That_can_Establish_Nitrogen-Fixing_Nodules_in_{—}Crotalia_{—}Longirostrata.pdf).

Suitable and commercially available Bradyrhizobium sp. (Arachis) strains especially for cowpea and peanut but also for soybean are Bradyrhizobium SEMIA 6144, SEMIA 6462 (=BR 3267) and SEMIA 6464 (=BR 3262) (deposited at FEPAGRO-MIRCEN, R. Gonsalves Dias, 570 Porto Alegre-RS, 90130-060, Brazil; see e.g. FEMS Microbiology Letters (2010) 303(2), 123-131; Revista Brasileira de Ciencia do Solo (2011) 35(3);739-742, ISSN 0100-0683).

The present invention also relates to mixtures wherein the at least one biopesticide II is selected from Bradyrhizobium sp. (Arachis) and further comprises a compound III, wherein compound III is selected from jasmonic acid or salts or derivatives thereof including cis-jasmone, preferably methyl-jasmonate or cis-jasmone.

The present invention also relates to mixtures, wherein the at least one biopesticide II is selected from Bradyrhizobium sp. (Lupine) (also called B. lupini, B. lupines or Rhizobium lupini). This mixture is especially suitable for use in dry beans and lupins.

Suitable and commercially available B. lupini strain is LL13 (isolated from Lupinus iuteus nodules from French soils; deposited at INRA, Dijon and Angers, France; http://agriculture.gouv.fr/IMG/pdf/ch20060216.pdf). This strain is especially suitable for lupins grown in Australia, North America or Europe, in particular in Europe.

Further suitable and commercially available B. lupini strains WU425 (isolated in Esperance, Western Australia from a non-Australian legume Ornthopus compressus), WSM4024 (isolated from lupins in Australia by CRS during a 2005 survey) and WSM471 (isolated from Ornithopus pinnatus in Oyster Harbour, Western Australia) are described e.g. in Palta J. A. and Berger J. B. (eds), 2008, Proceedings 12th International Lupin Conference, 14-18 Sep. 2008, Fremantle, Western Australia. International Lupin Association, Canterbury, New Zealand, 47-50, ISBN 0-86476-153-8:

http://www.lupins.org/pdf/conference/2008/Agronomy%20and%20Production/John%20Howieson%20and%20G%200Hara.pdf; Appl Environ Microbiol (2005) 71, 7041-7052 and Australian J. Exp. Agricult. (1996) 36(1), 63-70.

The present invention also relates to mixtures wherein the at least one biopesticide II is selected from Bradyrhizobium sp. (Lupine) (B. lupini) and further comprises a compound III, wherein compound III is selected from jasmonic acid or salts or derivatives thereof including cis-jasmone, preferably methyl-jasmonate or cis-jasmone.

The present invention also relates to mixtures, wherein the at least one biopesticide II is selected from Mesorhizobium sp. (meaning any Mesorhizobium species and/or strain), more preferably Mesorhizobium ciceri. These mixtures are particularly suitable in cowpea.

Suitable and commercially available M. sp. strains are e.g. M. ciceri CC1192 (=UPM 848, CECT 5549; from Horticultural Research Station, Gosford, Australia; collected in Israel from Cicer arietinum nodules; Can J Microbial (2002) 48, 279-284) and Mesorhizobium sp. strains WSM1271 (collected in Sardinia, Italy, from plant host Biserrula pelecinus), WSM 1497 (collected in Mykonos, Greece, from plant host Biserrula pelecinus), M. loti strains CC829 (commerical inoculant for Lotus pedunculatus and L. ulginosus in Australia, isolated from L. ulginosus nodules in USA) and SU343 (commercial inoculant for Lotus corniculatus in Australia; isolated from host nodules in USA) all of which are deposited at Western Australian Soil Microbiology (WSM) culture collection, Australia and/or CSIRO collection (CC), Canberra, Australian Capirtal Territory (see e.g. Soil Biol Biochem (2004) 36(8), 1309-1317; Plant and Soil (2011) 348(1-2), 231-243).

Suitable and commercially available M. loti strains are e.g. M. loti CC829 for Lotus pedunculatus.

The present invention also relates to mixtures wherein the at least one biopesticide II is selected from Bradyrhizobium sp. (Lupine) (B. lupini) and further comprises a compound III, wherein compound III is selected from jasmonic acid or salts or derivatives thereof including cis-jasmone, preferably methyl-jasmonate or cis-jasmone.

The present invention also relates to mixtures wherein the at least one biopesticide II is selected from Mesorhizobium huakuii, also referred to as Rhizobium huakuii (see e.g. Appl. Environ. Microbiol. 2011, 77(15), 5513-5516). These mixtures are particularly suitable in Astralagus, e.g. Astalagus sinicus (Chinese milkwetch), Thermopsis, e.g. Thermopsis luinoides (Goldenbanner) and alike.

Suitable and commercially available M. huakuii strain is HN3015 which was isolated from Astralagus sinicus in a rice-growing field of Southern China (see e.g. World J. Microbiol. Biotechn. (2007) 23(6), 845-851, ISSN 0959-3993).

The present invention also relates to mixtures wherein the at least one biopesticide II is selected from Mesorhizobium huakuii and further comprises a compound III, wherein compound III is selected from jasmonic acid or salts or derivatives thereof including cis-jasmone, preferably methyl-jasmonate or cis-jasmone.

The present invention also relates to mixtures, wherein the at least one biopesticide II is selected from Azospirillum amazonense, A. brasilense, A. lipoferum, A. irakense, A. halopraeferens, more preferably from A. brasilense, in particular selected from A. brasilense strains BR 11005 (SP 245) and AZ39 which are both commercially used in Brazil and are obtainable from EMBRAPA, Brazil. These mixtures are particularly suitable in soybean.

Humates are humic and fulvic acids extracted from a form of lignite coal and clay, known as leonardite. Humic acids are organic acids that occur in humus and other organically derived materials such as peat and certain soft coal. They have been shown to increase fertilizer efficiency in phosphate and micro-nutrient uptake by plants as well as aiding in the development of plant root systems.

Salts of jasmonic acid (jasmonate) or derivatives include without limitation the jasmonate salts potassium jasmonate, sodium jasmonate, lithium jasmonate, ammonium jasmonate, dimethylammonium jasmonate, isopropylammonium jasmonate, diolammonium jasmonate, diethtriethanolammonium jasmonate, jasmonic acid methyl ester, jasmonic acid amide, jasmonic acid methylamide, jasmonic acid-L-amino acid (amide-linked) conjugates (e.g., conjugates with L-isoleucine, L-valine, L-leucine, or L-phenylalanine), 12-oxo-phytodienoic acid, coronatine, coronafacoyl-L-serine, coronafacoyl-L-threonine, methyl esters of 1-oxo-indanoyl-isoleucine, methyl esters of 1-oxo-indanoyl-leucine, coronalon (2-[(6-ethyl-l-oxo-indane-4-carbonyl)-amino]-3-methyl-pentanoic acid methyl ester), linoleic acid or derivatives thereof and cis-jasmone, or combinations of any of the above.

According to one embodiment, the microbial pesticides embrace not only the isolated, pure cultures of the respective micro-organism as defined herein, but also its cell-free extract, its suspensions in a whole broth culture or as a metabolite-containing supernatant or a purified metabolite obtained from a whole broth culture of the microorganism or microorganism strain.

According to a further embodiment, the microbial pesticides embrace not only the isolated, pure cultures of the respective micro-organism as defined herein, but also a cell-free extract thereof or at least one metabolite thereof, and/or a mutant of the respective micro-organism having all the identifying characteristics thereof and also a cell-free extract or at least one metabolite of the mutant.

“Whole broth culture” refers to a liquid culture containing both cells and media.

“Supernatant” refers to the liquid broth remaining when cells grown in broth are removed by centrifugation, filtration, sedimentation, or other means well known in the art.

The term “metabolite” refers to any compound, substance or byproduct produced by a microorganism (such as fungi and bacteria) that has improves plant growth, water use efficiency of the plant, plant health, plant appearance, or the population of beneficial microorganisms in the soil around the plant activity.

The term “mutant” refers a microorganism obtained by direct mutant selection but also includes microorganisms that have been further mutagenized or otherwise manipulated (e.g., via the introduction of a plasmid). Accordingly, embodiments include mutants, variants, and or derivatives of the respective microorganism, both naturally occurring and artificially induced mutants. For example, mutants may be induced by subjecting the microorganism to known mutagens, such as N-methyl-nitrosoguanidine, using conventional methods.

According to the invention, the solid material (dry matter) of the biopesticides (with the exception of oils such as Neem oil, Tagetes oil, etc.) are considered as active components (e.g. to be obtained after drying or evaporation of the extraction medium or the suspension medium in case of liquid formulations of the microbial pesticides).

In accordance with the present invention, the weight ratios and percentages used herein for biological extract such as Quillay extract are based on the total weight of the dry content (solid material) of the respective extract(s).

For microbial pesticides, weight ratios and/or percentages refer to the total weight of a preparation of the respective biopesticide with at least 1×106 CFU/g (“colony forming units per gram total weight”), preferably with at least 1×108 CFU/g, even more preferably from 1×108 to 1×1012 CFU/g dry matter. Colony forming unit is measure of viable microbial cells, in particular fungal and bacterial cells. In addition, here CFU may also be understood as number of (juvenile) individual nematodes in case of (entomo-ipathogenic) nematode biopesticides, such as Steinernema feltiae.

Herein, microbial pesticides may be supplied in any physiological state such as active or dormant. Such dormant active component may be supplied for example frozen, dried, or lyophilized or partly desiccated (procedures to produce these partly desiccated organisms are given in WO2008/002371) or in form of spores.

Microbial pesticides used as organism in an active state can be delivered in a growth medium without any additional additives or materials or in combination with suitable nutrient mixtures.

According to a further embodiment, microbial pesticides are delivered and formulated in a dormant stage, more preferably in form of spores.

The total weight ratios of compositions, which comprise a microbial pesticide as component 2, can be determined based on the total weight of the solid material (dry matter) of component 1) and using the amount of CFU of component 2) to calclulate the total weight of component 2) with the following equation that 1×10⁹ CFU equals one gram of total weight of component 2).

According to one embodiment, the compositions, which comprise a microbial pesticide, comprise between 0.01 and 90% (w/w) of dry matter (solid material) of component 1) and from 1×10⁵ CFU to 1×10¹² CFU of component 2) per gram total weight of the composition.

According to another embodiment, the compositions, which comprise a microbial pesticide, comprise between 5 and 70% (w/w) of dry matter (solid material) of component 1) and from 1×10⁶ CFU to 1×10¹⁰ CFU of component 2) per gram total weight of the composition.

According to another embodiment, the compositions, wherein one component is a microbial pesticide, comprise between 25 and 70% (w/w) of dry matter (solid material) of component 1) and from 1×10⁷ CFU to 1×10⁹ CFU of component 2) per gram total weight of the composition.

In the case of mixtures comprising a microbial pesticide, the application rates preferably range from about 1×10⁶ to 5×10¹⁵ (or more) CFU/ha. Preferably, the spore concentration is about 1×107 to about 1×1011 CFU/ha. In the case of (entomopathogenic) nematodes as microbial pesticides (e.g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e.g. in the form of eggs, juvenile or any other live stages, preferably in an infective juvenile stage) per ha.

In the case of mixtures comprising microbial pesticides, the application rates with respect to plant propagation material preferably range from about 1×10⁶ to 1×10¹² (or more) CFU/seed. Preferably, the concentration is about 1×10⁶ to about 1×10¹¹ CFU/seed. In the case of microbial pesticides, the application rates with respect to plant propagation material also preferably range from about 1×10⁷ to 1×10¹⁴ (or more) CFU per 100 kg of seed, preferably from 1×10⁹ to about 1×10¹¹ CFU per 100 kg of seed.

The combined use of the compounds of formula (I) with other pesticides may result in potential synergistic effects. The examples of insecticidal mixing partners are provided with the intention to illustrate the possible combinations, but not to impose any limitation to the obtainable mixtures.

In the case that the mixing partner selected from list M is the insecticide to which the insect to be controlled is resistant, the mixture is suitable for controlling groups of insects which comprise resistant and non-resistant insects. In cases where it is not clear, whether the insect to be controlled is resistant or not, such a mixture is particularly useful.

In another embodiment of the invention, the compounds of formula (I), or their stereoisomers, salts, tautomers and N-oxides, may also be applied with fungicides as compound II.

The following list F of active substances, in conjunction with which the compounds according to the invention can be used, is intended to illustrate the possible combinations but does not limit them:

F.I) Respiration Inhibitors

F.I-1) Inhibitors of complex III at Qo site:

strobilurins: azoxystrobin, coumethoxystrobin, coumoxystrobin, dimoxystrobin, enestroburin, fluoxastrobin, kresoxim-methyl, mandestrobin, metominostrobin, orysastrobin, picoxystrobin, pyraclostrobin, pyrametostrobin, pyraoxystrobin, pyribencarb, triclopyricarb/chlorodincarb, tri-floxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methyl-acetamide;

oxazolidinediones and imidazolinones: famoxadone, fenamidone;

F.I-2) Inhibitors of complex II (e.g. carboxamides):

carboxanilides: benodanil, benzovindiflupyr, bixafen, boscalid, carboxin, fenfuram, fenhexamid, fluopyram, flutolanil, furametpyr, isofetamid, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide (fluxapyroxad), N-(4′-trifluoromethylthiobiphenyl-2-yl)-3 difluoromethyl-1-methyl-1H pyrazole-4-carboxamide, N-(2-(1,3,3-trimethyl-butyl)-phenyl)-1,3-dimethyl-5 fluoro-1H-pyrazole-4 carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 3-(trifluorometh [symbol] yl)-1,5-dimethyl-N-(1,1,3-trimethylindan-4-yl) [symbol] pyrazole-4-carboxamide, and 1,3,5-trimethyl-N-(1,1,3-trimethylindan-4-yl)pyrazole-4-carboxamide, N-(7-fluoro-1,1,3-trimethyl-indan-4-yl)-1,3-dimethyl-pyrazole-4-carbox [symbol] amide, N-[2-(2,4-dichlorophenyl)-2-methoxy-1-methyl-ethyl]-3-(difluoromethyl)-1-methylpyrazole-4-carboxamide;

F.I-3) Inhibitors of complex III at Qi site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate, 3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl2-methylpropanoate;

F.I-4) Other respiration inhibitors (complex I, uncouplers) diflumetorim; (5,8-difluoroquinazolin-4-yl)-{2-[2-fluoro-4-(4-trifluoromethylpyridin-2-yloxy)-phenyl]-ethyl}-amine; tecnazen; ametoctradin; silthiofam; nitrophenyl derivates: binapacryl, dinobuton, dinocap, fluazinam, ferimzone; nitrthalisopropyl, organometal compounds: fentin salts, such as fentin-acetate, fentin chloride or fentin hydroxide;

F.II) Sterol biosynthesis inhibitors (SBI fungicides)

F.II-1) C14 demethylase inhibitors (DMI fungicides, e.g. triazoles, imidazoles) triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, 1-[rel-(2S,3R)-3-(2-chlorophenyl)-2-(2,4-difluorophenyl)-oxiranylmethyl]-5-thiocyanato-1H-[1,2,4]triazole, 2-[rel-(2S,3R)-3-(2-chlorophenyl)-2-(2,4-difluorophenyl)-oxiranylmethyl]-2H-[1,2,4]triazole-3-thiol, 2-[2-chloro-4-(4-chlorophenoxy)phenyl]-1-(1,2,4-triazol-1-yl)pentan-2-ol, 1-[4-(4-chlorophenoxy)-2-(trifluoromethyl)phenyl]-1-cyclopropyl-2-(1,2,4-triazol-1-yl)ethanol, 2-[4-(4-chlorophenoxy)-2-(trifluoromethyl)phenyl]-1-(1,2,4-triazol-1-yl)butan-2-ol, 2-[2-chloro-4-(4-chlorophenoxy)phenyl]-1-(1,2,4-triazol-1-yl)butan-2-ol, 2-[4-(4-chlorophenoxy)-2-(trifluoromethyl)phenyl]-3-methyl-1-(1,2,4-triazol-1-yl)butan-2-ol, 2-[4-(4-chlorophenoxy)-2-(trifluoromethyl)phenyl]-1-(1,2,4-triazol-1-yl)propan-2-ol, 2-[2-chloro-4-(4-chlorophenoxy)phenyl]-3-methyl-1-(1,2,4-triazol-1-yl)butan-2-ol, 2-[4-(4-chlorophenoxy)-2-(trifluoromethyl)phenyl]-1-(1,2,4-triazol-1-yl)pentan-2-ol, 2-[4-(4-fluorophenoxy)-2-(trifluoromethyl)phenyl]-1-(1,2,4-triazol-1-yl)propan-2-ol;

imidazoles: imazalil, pefurazoate, oxpoconazole, prochloraz, triflumizole; pyrimidines, pyridines and piperazines: fenarimol, nuarimol, pyrifenox, triforine, [3-(4-chloro-2-fluoro-phenyl)-5-(2,4-difluorophenyl)isoxazol-4-yl]-(3-pyridyl)methanol;

F.II-2) Delta14-reductase inhitors (Amines, e.g. morpholines, piperidines)

morpholines: aldimorph, dodemorph, dodemorph-acetate, fenpropimorph, tridemorph; piperidines: fenpropidin, piperalin; spiroketalamines: spiroxamine;

F.II-3) Inhibitors of 3-keto reductase: hydroxyanilides: fenhexamid;

F.III) Nucleic acid synthesis inhibitors

F.III-1) RNA, DNA synthesis

phenylamides or acyl amino acid fungicides: benalaxyl, benalaxyl-M, kiralaxyl, metalaxyl, metalaxyl-M (mefenoxam), ofurace, oxadixyl;

isoxazoles and iosothiazolones: hymexazole, octhilinone;

F.III-2) DNA topisomerase inhibitors: oxolinic acid;

F.III-3) Nucleotide metabolism (e.g. adenosin-deaminase), hydroxy (2-amino)-pyrimidines: bupirimate;

F.IV) Inhibitors of cell division and or cytoskeleton

F.IV-1) Tubulin inhibitors: benzimidazoles and thiophanates: benomyl, carbendazim, fuberidazole, thiabendazole, thiophanate-methyl;

triazolopyrimidines: 5-chloro-7 (4-methylpiperidin-1-yl)-6-(2,4,6-trifluorophenyl)-[1,2,4]triazolo[1,5a]pyrimidine;

F.IV-2) Other cell division inhibitors

benzamides and phenyl acetamides: diethofencarb, ethaboxam, pencycuron, fluopicolide, zoxamide;

F.IV-3) Actin inhibitors: benzophenones: metrafenone; pyriofenone;

F.V) Inhibitors of amino acid and protein synthesis

F.V-1) Methionine synthesis inhibitors (anilino-pyrimidines)

anilino-pyrimidines: cyprodinil, mepanipyrim, nitrapyrin, pyrimethanil;

F.V-2) Protein synthesis inhibitors (anilino-pyrimidines)

antibiotics: blasticidin-S, kasugamycin, kasugamycin hydrochloride-hydrate, mildiomycin, streptomycin, oxytetracyclin, polyoxine, validamycin A;

F.VI) Signal transduction inhibitors

F.VI-1) MAP/Histidine kinase inhibitors (e.g. anilino-pyrimidines)

dicarboximides: fluoroimid, iprodione, procymidone, vinclozolin;

phenylpyrroles: fenpiclonil, fludioxonil;

F.VI-2) G protein inhibitors: quinolines: quinoxyfen;

F.VII) Lipid and membrane synthesis inhibitors

F.VII-1) Phospholipid biosynthesis inhibitors

organophosphorus compounds: edifenphos, iprobenfos, pyrazophos;

dithiolanes: isoprothiolane;

F.VII-2) Lipid peroxidation: aromatic hydrocarbons: dicloran, quintozene, tecnazene, tolclofosmethyl, biphenyl, chloroneb, etridiazole;

F.VII-3) Carboxyl acid amides (CAA fungicides)

cinnamic or mandelic acid amides: dimethomorph, flumorph, mandiproamid, pyrimorph;

valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;

F.VII-4) Compounds affecting cell membrane permeability and fatty acids:

1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, carbamates: propamocarb, propamocarb-hydrochlorid,

F.VII-5) Fatty acid amide hydrolase inhibitors: 1-[4-[4-[5-(2,6-difluorophenyl)-4,5-dihydro-3 isoxazolyl]-2-thiazolyl]-1-piperidinyl]-2-[5-methyl-3-(trifluoromethyl)-1H-pyrazol-1 yl]ethanone;

F.VIII) Inhibitors with Multi Site Action

F.VIII-1) Inorganic active substances: Bordeaux mixture, copper acetate, copper hydroxide, copper oxychloride, basic copper sulfate, sulfur;

F.VIII-2) Thio- and dithiocarbamates: ferbam, mancozeb, maneb, metam, methasulphocarb, metiram, propineb, thiram, zineb, ziram;

F.VIII-3) Organochlorine compounds (e.g. phthalimides, sulfamides, chloronitriles):

anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitrophenyl)-N-ethyl-4-methyl-benzenesulfonamide;

F.VIII-4) Guanidines and other: guanidine, dodine, dodine free base, guazatine, guazatine-acetate, iminoctadine, iminoctadine-triacetate, iminoctadine-tris(albesilate), dithianon, 2,6-dimethyl-1H,5H-[1,4]dithiino[2,3-c:5,6-c′]dipyrrole-1,3,5,7(2H,6H)-tetraone;

F.VIII-5) Ahtraquinones: dithianon;

F.IX) Cell wall synthesis inhibitors

F.IX-1) Inhibitors of glucan synthesis: validamycin, polyoxin B;

F.IX-2) Melanin synthesis inhibitors: pyroquilon, tricyclazole, carpropamide, dicyclomet, fenoxanil;

F.X) Plant defence inducers

F.X-1) Salicylic acid pathway: acibenzolar-S-methyl;

F.X-2) Others: probenazole, isotianil, tiadinil, prohexadione-calcium; phosphonates: fosetyl, fosetyl-aluminum, phosphorous acid and its salts;

F.XI) Unknown mode of action:bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxathiapiprolin, 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-fluoro-6-(prop-2-yn-1-yl-oxy)phenyl]-4,5-dihydro-1,2-oxazol-3-yl}-1,3-thi [symbol] azol-2-yl)piperidin-1-yl]ethanone, 2[3,5-bis(difluoromethyl)-1H-pyrazol-1-yl]-1-[4-(4-{5-[2-chloro-6-(prop-2-yn-1-yl-oxy)phenyl]-4,5-dihydro-1,2-oxazol-3-yl}-1,3-thiazol-2 yl)piperidin-1-yl]ethanone, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl) cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide, ethyl (Z) 3 amino-2-cyano-3-phenyl-prop-2-enoate, tert-butyl N-[6-[[(Z)-[(1-methyltetrazol-5-yl)-phenyl-methylene]amino]oxymethyl]-2-pyridyl]carbamate, pentyl N-[6-[[(Z)-[(1-methyltetrazol-5-yl)-phenyl-methylene]amino]oxymethyl]-2-pyridyl]carbamate, 2-[2-[(7,8-difluoro-2-methyl-3-quinolyl)oxy]-6-fluoro-phenyl]propan-2-ol, 2-[2-fluoro-6-[(8-fluoro-2-methyl-3-quinolyl)oxy]phenyl]propan-2-ol, 3-(5-fluoro-3,3,4,4-tetramethyl-3,4-dihydroiso [symbol] quinolin-1-yl)quinoline, 3-(4,4-difluoro-3,3-dimethyl-3,4-dihydroisoquinolin-1-yl) [symbol] quinoline, 3-(4,4,5-trifluoro-3,3-dimethyl-3,4-dihydroisoquinolin-1-yl)quinoline;

F.XI) Growth regulators: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6-benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;

F.XIII) Biopesticides

F.XIII-1) Microbial pesticides with fungicidal, bactericidal, viricidal and/or plant defense activator activity: Ampelomyces quisqualis, Aspergillus flavus, Aureobasidium pullulans, Bacillus amyloliquefaciens, B. mojavensis, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Fusarium oxysporum, Clonostachys rosea f. catenulate (also named Gliocladium catenulatum), Gliocladium roseum, Metschnikowia fructicola, Microdochium dimerum, Paenibacillus polymyxa, Pantoea agglomerans, Phlebiopsis gigantea, Pseudozyma flocculosa, Pythium oligandrum, Sphaerodes mycoparasitica, Streptomyces lydicus, S. violaceusniger, Talaromyces flavus, Trichoderma asperellum, T. atroviride, T. fertile, T. gamsii, T. harmatum; mixture of T. harzianum and T. viride; mixture of T. polysporum and T. harzianum; T. stromaticum, T. virens (also named Gliocladium virens), T. viride, Typhula phacorrhiza, Ulocladium oudema, U. oudemansii, Verticillium dahlia, zucchini yellow mosaic virus (avirulent strain);

F.XIII-2) Biochemical pesticides with fungicidal, bactericidal, viricidal and/or plant defense activator activity: chitosan (hydrolysate), jasmonic acid or salts or derivatives thereof, laminarin, Menhaden fish oil, natamycin, Plum pox virus coat protein, Reynoutria sachlinensis extract, salicylic acid, tea tree oil;

F.XIII-3) Microbial pesticides with plant stress reducing, plant growth regulator, plant growth promoting and/or yield enhancing activity: Azospirillum amazonense A. brasilense, A. lipoferum, A. irakense, A. halopraeferens, Bradyrhizobium sp., B. japonicum, Glomus intraradices, Mesorhizobium sp., Paenibacillus alvei, Penicillium bilaiae, Rhizobium leguminosarum bv. phaseolii, R. I. trifolii, R. I. bv. viciae, Sinorhizobium meliloti;

F.XIII-4) Biochemical pesticides with plant stress reducing, plant growth regulator and/or plant yield enhancing activity: abscisic acid, aluminium silicate (kaolin), 3-decen-2-one, homobrassinlide, humates, lysophosphatidyl ethanolamine, polymeric polyhydroxy acid, Ascophyllum nodosum (Norwegian kelp, Brown kelp) extract and Ecklonia maxima (kelp) extract.

The commercially available compounds II of the group F listed above may be found in The Pesticide Manual, 15th Edition, C. D. S. Tomlin, British Crop Protection Council (2011) among other publications. Their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EPA 141 317; EP-A 152 031; EP-A 226 917; EPA 243 970; EPA 256 503; EP-A 428 941; EP-A 532 022; EP-A 1 028 125; EP-A 1 035 122; EPA 1 201 648; EPA 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657). The biopesticides of group F.XIII are disclosed above in the paragraphs about biopesticides from group M.Y

The compounds I and the one or more compound(s) II are usually applied in a weight ratio of from 5000:1 to 1:5000, preferably from 1000:1 to 1:1000, preferably from 625:1 to 1:625, preferably 500:1 to 1:100, preferably from 100:1 to 1:100 preferably from 20:1 to 1:50, preferably from 20:1 to 1:20, preferably from 10:1 to 1:10, in particular from 5:1 to 1:20, in particular from 5:1 to 1:10, in particular from 5:1 to 1:5.

Depending on the desired effect, the application rates of the mixtures according to the invention are from 5 g/ha to 2000 g/ha, preferably from 0.5 g/ha to 1000 g/ha, preferably from 1 to 750 g/ha, in particular from 5 to 500 g/ha.

The mixtures, as described above for the methods and uses of the invention, may optionally comprise more than one compound II as combination partner. The mixture may comprise, apart from the compound of formula (I), one active compound II (binary mixture), two active compounds II (ternary mixture), three active compounds II (4-way mixture), four active compounds II (5-way mixture).

All explanations and preferences as described for the methods and uses for the compound of formula (I) or a stereoisomer, salt, tautomer or N-oxide thereof, also apply for the methods and uses wherein a mixture is used, e.g. the explanations and preferences regarding pests to be controlled, insecticides to which the target organism is resistant, formulations, applications, and also the compounds of formula (I) itself.

Applications

In the methods and uses according to the invention, the application of the the compounds of the present invention, including their stereoisomers, salts, tautomers and N-oxides, is preferably as explained in the following.

The animal pest (also referred to as “invertebrate pest”), i.e. the insects, arachnids and nematodes, the plant, soil or water in which the plant is growing can be contacted with the present compounds of formula I or composition(s) comprising them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with an insecticidally effective amount of the active compounds. The application can be carried out both before and after the infection of the plants, plant propagation materials, such as seeds, soil, surfaces, materials or rooms by the insects.

The present invention also includes a method of combating animal pests which comprises contacting the animal pests, their habitat, breeding ground, food supply, cultivated plants, seed, soil, area, material or environment in which the animal pests are growing or may grow, or the materials, plants, seeds, soils, surfaces or spaces to be protected from animal attack or infestation with a pesticidally effective amount of a mixture of at least one active compound I.

Moreover, animal pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, the genetic material of which has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s) (oligo- or polypeptides) for example by glycosylation or polymer additions such as prenylated, acetylated or farnesylated moieties or PEG moieties(e.g. as disclosed in Biotechnol Prog. 2001 July-August; 17(4):720-8., Protein Eng Des Sel. 2004 January; 17(1):57-66, Nat Protoc. 2007; 2(5): 1225-35, Curr Opin Chem Biol. 2006 October; 10(5):487-91. Epub 2006 Aug 28, Biomaterials. 2001 March; 22(5):405-17, Bioconjug Chem. 2005 January-February; 16(1):113-21).

The term “cultivated plants” is to be understood also including plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e. g. U.S. Pat. No. 6222100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e. g. WO 92/00377); glutamine synthetase (GS) inhibitors, such as glufosinate (see e. g. EP-A-0242236, EP-A-242246) or oxynil herbicides (see e. g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e. g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e. g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e. g. bio mass production, grain yield, starch content, oil content or protein content), tolerance to drought, salinity or other growth-limiting environ-mental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e. g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e. g. Amflora® potato).

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 25 g to 600 g per hectare, more desirably from 50 g to 500 g per hectare.

The compounds of formula I are effective through both contact (via soil, glass, wall, bed net, carpet, plant parts or animal parts), and ingestion (bait, or plant part).

The compounds of the invention may also be applied against non-crop insect pests, such as ants, termites, wasps, flies, mosquitos, crickets, or cockroaches. For use against said non-crop pests, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methyl-neodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

Seed Treatment

In the methods and uses according to the invention, the compounds of formula I are also suitable for the treatment of seeds in order to protect the seed from insect pest, in particular from soil-living insect pests and the resulting plant's roots and shoots against soil pests and foliar insects.

In the methods and uses according to the invention, the compounds of formula I are also suitable for the treatment of soil. Soil application techniques and soil application methods according to the present invention, are methods wherein the active compound(s) are applied by drenching the soil, applied by drip irrigation, applied by soil injection. Another soil application technique in the sense of the present invention is a method, wherein the active compound(s) are applied by dipping roots, tubers or bulbs.

An alternative method of soil application technique is that the active compound(s) are applied with drip application systems. In the case of soil treatment or of application to the pests dwelling place or nest, the quantity of active ingredient ranges from 0.0001 to 500 g per 100 m², preferably from 0.001 to 20 g per 100 m².

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected from piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A 242 236, EP-A 242 246) (WO 92/00377) (EP-A 257 993, U.S. Pat. No. 5,013,659) or in transgenic crop plants, for example cotton, with the capability of producing Bacillus thuringiensis toxins (Bt toxins) which make the plants resistant to certain pests (EP-A 142 924, EP-A 193 259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

D Emulsions (EW, EO, ES)

E Suspensions (SC, OD, FS)

F Water-dispersible granules and water-soluble granules (WG, SG)

G Water-dispersible powders and water-soluble powders (WP, SP, WS)

H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typcially, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight. Seed Treatment formulations may additionally also comprise binders and optionally colorants. Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108. Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

### EXAMPLES

The present invention is now illustrated in further detail by the following examples.

A. Chemistry

The compounds I of formula I can be accomplished according to standard methods of organic chemistry, e.g. by the methods or working examples described in WO 2007/006670, PCT/EP2012/065650, PCT/EP2012/065651.

The characterization can be done by coupled High Performance Liquid Chromatography/mass spectrometry (HPLC/MS), by NMR or by their melting points.

Method A: Analytical HPLC column: RP-18 column Chromolith Speed ROD from Merck KgaA (Germany). Elution: acetonitrile+0.1% trifluoroacetic acid (TFA)/water+0.1% trifluoroacetic acid (TFA) in a ratio of from 5:95 to 95:5 in 5 minutes at 40° C.

Method B: Analytical UPLC column: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.20 min; flow: 0.8-1.0 mL/min in 1.50 minutes at 60° C.

MS-method: ESI positive.

¹H-NMR. The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singulett.

### PREPARATION EXAMPLES

logP determinations were performed via capillary electrophorese on a cePro9600™ from CombiSep.

Starting Materials

6,8-dichloro-1H-benzo[d][1,3]oxazine-2,4-dione and 6-chloro-8-methyl-1H-3,1-benzoxazine-2,4-dione were prepared according to WO 2007/43677.

S,S-Diisopropyl-S-aminosulfonium 2,4,6-trimethylphenylsulfonat was prepared according to Y. Tamura et al, Tetrahedron 1975, 31, 3035-3040.

2-(3-Chloropyridin-2-yl)-5-bromo-2H-pyrazole-3-carbonyl chloride was prepared according to WO 2007/24833.

### Preparation Examples P.1 to P.4

### Example P.1

### S,S-Dimethyl Sulfinium Sulfate

To a solution of sodium methylate (15.76 g of a 30% solution in methanol, 87.54 mmol, 1.100 equiv.) in methanol (60 mL) was added dimethyl sulphide (5.44 g, 6.40 mL, 87.6 mmol, 1.10 equiv.) at −5-0° C. To this mixture was added a pre-cooled solution (−20° C.) of hydroxylamine-O-sulfonic acid (9.00 g, 79.6 mmol) in methanol (60 mL) and the internal temperature was maintained at −5-0° C. After stirring at room temperature overnight, all solids were removed by filtration. The filtrate was concentrated in vacuo and the residue was triturated with acetonitrile (50 mL) to yield the title compound (7.88 g, 39%).

The following compounds were prepared by analogy to example P.1:

S,S-diethyl sulfinium sulfate

S-ethyl-S-isopropyl sulfinium sulfate

S,S-diisopropyl sulfinium sulfate

S,S-bis(2-cyclopropylmethyl)sulfinium sulfate

S,S-bis(2-cyclopropylethyl)sulfinium sulfate

S,S-bis(cyclobutylmethyl)sulfinium sulfate

S,S-bis(cyclopentylmethyl)sulfinium sulfate

S-cyclopropylmethyl-S-ethyl sulfinium sulfate

S-(2-cyclopropylethyl)-S-ethyl sulfinium sulfate

S-(2-cyclopropylethyl)-S-isopropyl sulfinium sulfate

S-(1-cyclopropylethyl)-S-isopropyl sulfinium sulfate

S-cyclobutylmethyl-S-ethyl sulfinium sulfate

S-cyclopentylmethyl-S-ethyl sulfinium sulfate

S-cyclopropylmethyl-S-isopropyl sulfinium sulfate

S-cyclobutylmethyl-S-isopropyl sulfinium sulfate

S-cyclopentylmethyl-S-isopropyl sulfinium sulfate

S,S-di-n-propyl sulfinium sulfate

S-vinyl-S-ethyl sulfinium sulfate

### Example P.2

### 8-Bromo-6-chloro-1H-benzo[d][1,3]oxazine-2,4-dione

To a solution of 2-amino-3-bromo-5-chlorobenzoic acid (10.0 g, 39.9 mmol) in dioxane (170 mL) was added phosgene (20% in toluene, 42.0 mL, 79.9 mmol) over a period of 15 mins. The reaction was stirred at ambient temperature for 48 h and then concentrated in vacuo. The resulting solid was crushed and further dried in vacuo to yield the desired product (12.6 g, 114%) which was used in the subsequent step without further purification.

The following compounds were prepared by analogy to example P.2:

6,8-dichloro-1H-benzo[d][1,3]oxazine-2,4-dione,

6,8-dibromo-1H-benzo[d][1,3]oxazine-2,4-dione,

6-Bromo-8-chloro-1H-benzo[d][1,3]oxazine-2,4-dione,

8-Bromo-6-chloro-1H-benzo[d][1,3]oxazine-2,4-dione,

6-chloro-8-methyl-1H-benzo[d][1,3]oxazine-2,4-dione,

6-bromo-8-methyl-1H-benzo[d][1,3]oxazine-2,4-dione,

6-cyano-8-methyl-1H-benzo[d][1,3]oxazine-2,4-dione,

6-chloro-8-trifluoromethyl-1H-benzo[d][1,3]oxazine-2,4-dione,

8-chloro-6-trifluoromethyl-1H-benzo[d][1,3]oxazine-2,4-dione,

6-bromo-8-trifluoromethyl-1H-benzo[d][1,3]oxazine-2,4-dione,

8-bromo-6-trifluoromethyl-1H-benzo[d][1,3]oxazine-2,4-dione,

8-chloro-6-cyano-1H-benzo[d][1,3]oxazine-2,4-dione,

6-chloro-8-methoxy-1H-benzo[d][1,3]oxazine-2,4-dione,

6-chloro-8-cyclopropyl-1H-benzo[d][1,3]oxazine-2,4-dione,

6-chloro-8-ethyl-1H-benzo[d][1,3]oxazine-2,4-dione,

6-difluoromethoxy-8-methyl-1H-benzo[d][1,3]oxazine-2,4-dione,

6-cyano-8-methoxy-1H-benzo[d][1,3]oxazine-2,4-dione,

6-fluoro-8-methyl-1H-benzo[d][1,3]oxazine-2,4-dione,

6-iodo-8-methyl-1H-benzo[d][1,3]oxazine-2,4-dione,

6-nitro-8-methyl-1H-benzo[d][1,3]oxazine-2,4-dione,

6-(5-chloro-2-thienyl)-8-methyl-1H-benzo[d][1,3]oxazine-2,4-dione,

6-(3-pyrazol-1H-yl)-8-methyl-1H-benzo[d][1,3]oxazine-2,4-dione,

6-(3-isoxazolyl)-8-methyl-1H-benzo[d][1,3]oxazine-2,4-dione,

6-(hydroxyiminomethyl)-8-methyl-1H-benzo[d][1,3]oxazine-2,4-dione,

6-(methoxyiminomethyl)-8-methyl-1H-benzo[d][1,3]oxazine-2,4-dione,

6-(dimethylhydrazonomethyl)-8-methyl-1H-benzo[d][1,3]oxazine-2,4-dione and

6-(2,2,2-trifluoroethylhydrazonomethyl)-8-methyl-1H-benzo[d][1,3]oxazine-2,4-dione.

### Example P.3

### 1-(3-chloro-2-pyridyl)-3-trifluoromethyl-1H-pyrazol

a) 2.71 kg of 1,1,1-trifluoro-4-methoxy-but-3-en-2-one, 2,44 kg of ethanol and 3.10 kg of water were charged into a reaction vessel. 20 ml of concentrated hydrochloric acid and 0.80 kg of hydrazine hydrate were successively added and the mixture was heated to reflux for 4 h. The mixtures was allowed to cool and neutralized by addition of 10% aqueous NaOH to about pH 4-5. Then the mixture was evaporated. Toluene was added and the mixture was again evaporated to yield 2 kg of raw 3-trifluoromethylpyrazole with a purity of >85%.

b) 1.72 kg (10.75 mol) of the raw 3-trifluoromethylpyrazole obtained in step a), 1.75 kg (11.83 mol) of 2,3-dichloropyridine and 4.73 kg of dimethyl formamide were charged to a reaction vessel. 2.97 kg (21.50 mol) of potassium carbonate were added, the mixture was heated to 120° C. with stirring and kept at 120-125° C. for further 3 h. The reaction mixtures was cooled to 25° C. and poured into 20 l of water. The thus obtained mixture was extracted twice with 5 L of tert.-butylmethyl ether. The combined organic phases were washed with 4 l of water and then evaporated to dryness. Toluene was added and the mixture was again evaporated to dryness. Thereby, the 2.7 kg of the title compound was obtained (purity >75% as determined by GC; yield 81.5%). The product can be purified by distillation.

¹H-NMR (400 MHz, CDCl₃): δ [delta]=6.73 (d, 1H), 7.38 (d, 1H), 7.95 (m, 1H), 8.14 (m, 1H), 8.46 (m, 1H).

### Example P.4

### 2-(3-Chloropyridin-2-yl)-5-trifluoromethyl-2H-pyrazole-3-carbonyl chloride

In a reaction vessel equipped with a thermometer, septum, nitrogen inlet and stirring bar, 10.0 g (40.4 mmol) of 1-(3-chloro-2-pyridyl)-3-trifloromethyl-1H-pyrazole were dissolved in 50 ml of dry dimethoxyethane. By means of a syringe, 40.4 ml of a 2 M solution (80.8 mmol, 2.0 equiv.) of isopropyl magnesium chloride in tetrahydrofuran were added dropwise with stirring, while cooling the vessel with an ice bath and keeping the internal temperature at about 5° C. The mixture was stirred for further 2 hours at 5° C. Then the ice-bath was removed and carbon dioxide was bubbled through mixture causing an increase of the temperature up to 28° C. After 10 minutes, the exothermic reaction has ceased, and, the mixture was cooled and all volatiles were removed by evaporation. The residue containing the carboxylate compound I-A was taken up in 50 mL of dichloromethane and one drop of dry DMF was added. To this mixture, 14.41 g (121.2 mmol, 3.0 equiv.) of thionyl chloride were added and heated to reflux for 3 hours. After cooling, the resulting precipitate was removed by filtration and the mother liquid was concentrated in vacuum to obtain 13.0 g of the title compound (purity >85%, yield 100%) which was used in the next step without further purification.

¹H-NMR (400 MHz, CDCl₃): δ[delta]=7.43-7.54 (m, 2H), 7.93 (d, 1H), 8.52 (m, 1H).

### Example P.5

### 2-amino-5-chloro-N-(dimethyl-λ⁴-sulfanylidene)-3-methyl-benzamide

To a solution of 6-chloro-8-methyl-1H-3,1-benzoxazine-2,4-dione (3.00 g, 12.8 mmol) in dichloromethane (40 mL) was added dimethyl sulfinium sulfate (2.25 g, 8.93 mmol, 0.70 equiv.) and potassium tert-butylate (1.58 g, 14.0 mmol, 1.10 equiv.) at room temperature. The mixture was stirred for 1.5 h, upon which water was added and the layers were separated. The aqueous layer was extracted with dichloromethane, combined organic layers were dried over sodium sulphate and concentrated in vacuo. The residue was purified by flash-chromatography on silica gel to yield the title compound (2.63 g, 84%).

Characterization by HPLC-MS: 1.855 min, M=245.00.

### Example P.6

### 2-amino-5-chloro-N-(bis-2-methylpropyl-λ⁴-sulfanylidene)-3-methyl-benzamide

To a solution of 6-chloro-8-methyl-1H-3,1-benzoxazine-2,4-dione (3.00 g, 12.8 mmol) in dichloromethane (40 mL) was added bis-2-methylpropyl sulfinium sulfate (3.76 g, 8.93 mmol, 0.70 equiv.) and potassium tert-butylate (1.58 g, 14.0 mmol, 1.10 equiv.) at room temperature. The mixture was stirred for 1.5 h, upon which water was added and the layers were separated. The aqueous layer was extracted with dichloromethane, combined organic layers were dried over sodium sulphate and concentrated in vacuo. The residue was purified by flash-chromatography on silica gel to yield the title compound (2.89 g, 69%).

Characterization by ¹H-NMR (400 MHz, DMSO-d₆): δ[delta]=1.04 (m, 12H), 2.06 (s, 3H), 2.96 (m, 2H), 3.01 (m, 2H), 6.62 (br. s, 2H), 7.03 (s, 1H), 7.72 (s, 1H).

### Example P.7

### 2-amino-5-chloro-N-(diethyl-λ⁴-sulfanylidene)-3-methyl-benzamide

To a solution of 6-chloro-8-methyl-1H-3,1-benzoxazine-2,4-dione (2 g, 0.01 mol) in anhydrous propylene carbonate (30 mL) was added bis-2-ethyl sulfinium sulfate (2.04 g, 0.01 mol, 0.70 equiv.) and triethyl amine (1.38 mL, 1.0 g g, 0.01 mol, 1.05 equiv.) at room temperature. The mixture was stirred for 4.5 h, and then added dropwise to ice-water. The mixture was extracted with dichloromethane and the combined organic layers were dried over sodium sulphate and concentrated in vacuo. The residue was triturated with ether to yield the title compound (1.43 g, 55%).

Characterization by ¹H-NMR (400 MHz, CDCl₃): δ[delta]=1.39 (t, 6H), 2.13 (s, 3H), 3.02 (q, 4H), 5.95 (br. S, 2H), 7.01 (s, 1H), 7.98 (s, 1H).

### Example P.8

### 2-amino-3,5-dichloro-N-(bis-2-methylpropyl-λ⁴-sulfanylidene)-benzamide

The title compound was prepared by analogy to the method of example P.6

Yield: 60%

Characterization by ¹H-NMR (400 MHz, DMSO-d₆): δ[delta]=1.23 (d, 6H), 1.38 (d, 6H), 3.42 (m, 2H), 7.02 (br. s, 2H), 7.41 (s, 1H), 7.95 (s, 1H).

### Example P.9

### 2-amino-3,5-dibromo-N-(bis-2-methylpropyl-λ⁴-sulfanylidene)-benzamide

The title compound was prepared by analogy to the method of example P.6

Yield: 66%

Characterization by HPLC-MS: 3.409 min, m/z=410.90 (Method A)

Preparation of the compounds of formula IA-1 (Examples 1 to 4)

### Example 1

### 2-(3-chloro-2-pyridyl)-N-[2,4-dichloro-6-[(diethyl-λ⁴-sulfanylidene)carbamoyl]phenyl]-5-(trifluoromethyl)pyrazole-3-carboxamide (Compound I-16)

To a suspension of potassium carbonate (8.08 g, 58.5 mmol, 1.50 equiv) and 2-amino-3,5-dichloro-N-(diethyl-λ⁴-sulfanylidene)benzamide (11.43 g, 38.98 mmol) in acetonitrile (100 mL) was added a solution of 2-(3-chloro-2-pyridyl)-5-(trifluoromethyl)pyrazole-3-carbonyl chloride (15.8 g, 43.31 mmol, 1.10 equiv.) in acetonitrile (50 mL) at room temperature. After 6 h at this temperature, the solids were filtered off. The resulting filtrate was washed with water and dried over Na₂SO₄. After filtration, the filtrate was concentrated in vacuum and the resulting solids were crystallized from diisopropyl ether to yield the title compound (19.53 g, 88%).

Characterization by ¹H-NMR (400 MHz, DMSO-d₆):

δ[delta]=1.13 (t, 6H), 2.91 (m, 2H), 3.08 (m, 2H), 7.67 (dd, 1H), 7.77 (s, 2H), 7.89 (s, 1H), 8.22 (d, 1H), 8.51 (d, 1H), 10.73 (s, 1H).

### Example 2

### Synthesis of 2-(3-chloro-2-pyridyl)-N-[2,4-dichloro-6-[(bis-2-propyl-λ⁴-sulfanylidene)carbamoyl]phenyl]-5-(trifluoromethyl)pyrazole-3-carboxamide (Compound (I-26)

To a suspension of potassium carbonate (0.892 g, 6.46 mmol, 1.10 equiv) and 2-amino-3,5-dichloro-N-(bis-2-propyl-λ⁴-sulfanylidene)benzamide (2.05 g, 5.87 mmol) in toluene (30 mL) was added a solution of 2-(3-chloro-2-pyridyl)-5-(trifluoromethyl)pyrazole-3-carbonyl chloride (2.02 g, 5.87 mmol, 1.00 equiv.) in toluene (20 mL) at 60° C. After 45 min at this temperature, the mixture was cooled and water was added. The resulting precipitate was collected by filtration, washed with water and toluene and dried to obtain the title compound (3.07 g, 84%).

Characterization by HPLC-MS: 1.395 min, M=602.1 (Method B)

Characterization by ¹H-NMR (400 MHz, DMSO-d₆):

δ[delta]=1.18 (d, 6H), 1.22 (d, 6H), 3.30 (m, 2H), 7.68 (dd, 1H), 7.75 (m, 2H), 7.81 (s, 1H), 8.21 (d, 1H), 8.54 (d, 1H), 10.76 (s, 1H).

### Example 3

### Synthesis of 2-(3-chloro-2-pyridyl)-N-[2-methyl-4-chloro-6-[(bis-2-propyl-λ⁴-sulfanylidene)carbamoyl]phenyl]-5-(trifluoromethyl)pyrazole-3-carboxamide (Compound I-21)

To a suspension of potassium carbonate (126.01 g, 911.76 mmol, 1.30 equiv) and 2-amino-3-methyl-5-chloro-N-(bis-2-propyl-λ⁴-sulfanylidene)benzamide (211 g, 701 mmol) in dichloromethane (300 mL) was added a solution of 2-(3-chloro-2-pyridyI)-5-(trifluoromethyl)pyrazole-3-carbonyl chloride (256.78 g, 771.49 mmol, 1.10 equiv.) in dichloromethane (200 mL) at room temperature. After 2 h at this temperature, the solids were filtered off. The resulting filtrate was washed with water and dried over Na₂SO₄. After filtration, the filtrate was concentrated in vacuum and the resulting solids were crystallized from diisopropyl ether to yield the title compound (344.2 g, 85%).

Characterization by HPLC-MS: 1.303 min, M=574.3 (Method B)

Characterization by ¹H-NMR (400 MHz, DMSO-d₆): δ[delta]=1.20 (d, 6H), 1.30 (d, 6H), 2.15 (s, 3H), 3.30 (m, 2H), 7.41 (s, 1H), 7.62 (m, 2H), 7.80 (s, 1H), 8.22 (d, 1H), 8.52(d, 1H), 10.88 (s, 1H).

### Example 4a

### 2-(3-chloro-2-pyridyl)-N-[2-methyl-4-chloro-6-[(diethyl-λ⁴-sulfanylidene)carbamoyl]phenyl]-5-(trifluoromethyl)pyrazole-3-carboxamide (Compound I-11)

To a suspension of potassium carbonate (0.71 g, 10 mmol, 1.3 equiv) and 2-amino-3-methyl-5-chloro-N-(diethylλ⁴-sulfanylidene)benzamide (1.42 g, 3.96 mmol) in propylene carbonate (20 mL) was added a solution of 2-(3-chloro-2-pyridyl)-5-(trifluoromethyl)pyrazole-3-carbonyl chloride (1.35 g, 4.35 mmol, 1.10 equiv.) in propylene carbonate (10 mL) at room temperature. After 24 h at this temperature, the mixture was poured onto water and spiked with ethanol under vigorous stirring. The resulting solids were collected by filtration and contained pure title compound (1.57 g, 73%).

Characterization by HPLC-MS: 1.19 min, m/z 546.1 (M+H)+; (Method B)

Characterization by ¹H-NMR (500 MHz, DMSO) [delta]: 10.87 (s, 1H), 8.53 (d, 1H), 8.22 (d, 1H), 7.75 (s, 1H), 7.65 (m, 2H), 7.40 (s, 1H), 3.09 (m, 2H), 2.92 (m, 2H) 1.15 (m, 6H).

### Example 4b

### 2-(3-chloro-2-pyridyl)-N-[2-methyl-4-chloro-6-[(diethyl-λ⁴-sulfanylidene)carbamoyl]phenyl]-5-(trifluoromethyl)pyrazole-3-carboxamide (Compound I-11)

To a solution of 2-(3-chloro-2-pyridyl)-5-(trifluoromethyl)pyrazole-3-carbonyl chloride (150 g, 435 mmol) in acetonitrile (900 mL) at room temperature was added potassium carbonate (59 g, 427 mmol). A solution of 2-amino-5-chloro-N-(diethyl-sulfanylidene)-3-methyl-benzamide (117 g, 427 mmol) in acetonitrile (100 mL) was added dropwise within 1 hour while maintaining a reaction temperature of 25-28° C. with occasional cooling (slightly exothermic reaction). The mixture was stirred for 16 hours at room temperature. The reaction mixture was then poured on ice-water mixture (5 L) and the pH was adjusted to 7-8 with concentrated HCl. The mixture stirred for an additional 2 hours. The light brown solid was filtered, washed with water and dried under air to give the crude product (229 g).

3 combined batches of crude product (789 g) were suspended in acetonitrile (2.6 L) and dissolved upon heating at 60° C. After 1 hour of stirring at 60° C. the solution was cooled by means of an ice-bath and the thereby formed solid was filtered off. The mother-liquor was concentrated to 300 mL and cooled with ice-bath. Thereby additional solid formed was filtered. The combined solids were washed with cold acetonitrile and dried at 50° C. in a vacuum-oven over night to give the title product (703 g, 89%) as a crystalline white solid.

By the methods described in examples 1 to 4 or analogy therof, the compounds of formula (IA-1) summarized in table C were prepared:

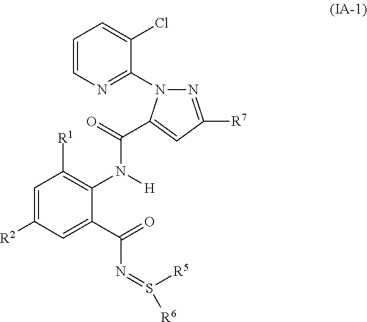

TABLE C

| | R¹ | R² | R⁷ | R⁵ | R⁶ | MS | RT[min] | m/z |
|---|---|---|---|---|---|---|---|---|
| I-1 | Me | Cl | CF₃ | CH₃ | CH₃ | logP: 2.9 [pH = 10.0]; m.p: 182° C. | | |
| I-2 | Me | Cl | CHF₂ | CH₃ | CH₃ | B | 1.06 | 500.2 |
| I-3 | Me | Cl | Br | CH₃ | CH₃ | A | 3.067 | 529.95 |
| I-4 | Me | Cl | Cl | CH₃ | CH₃ | | | |
| I-5 | Me | Cl | CN | CH₃ | CH₃ | | | |
| I-6 | Cl | Cl | CF₃ | CH₃ | CH₃ | A | 3.372 | 539.95 |
| I-7 | Cl | Cl | CHF₂ | CH₃ | CH₃ | B | 1.062 | 520.2 |
| I-8 | Cl | Cl | Br | CH₃ | CH₃ | A | 3.015 | 549.80 |
| I-9 | Cl | Cl | Cl | CH₃ | CH₃ | | | |
| I-10 | Cl | Cl | CN | CH₃ | CH₃ | | | |
| I-11 | Me | Cl | CF₃ | C₂H₅ | C₂H₅ | B | 1.207 | 546.1 |
| I-12 | Me | Cl | CHF₂ | C₂H₅ | C₂H₅ | B | 1.134 | 528.2 |
| I-13 | Me | Cl | Br | C₂H₅ | C₂H₅ | A | 3.309 | 557.95 |
| I-14 | Me | Cl | Cl | C₂H₅ | C₂H₅ | | | |
| I-15 | Me | Cl | CN | C₂H₅ | C₂H₅ | B | 1.098 | 503.3 |
| I-16 | Cl | Cl | CF₃ | C₂H₅ | C₂H₅ | A | 3.450 | 565.90 |
| I-17 | Cl | Cl | CHF₂ | C₂H₅ | C₂H₅ | B | 1.144 | 549.9 |
| I-18 | Cl | Cl | Br | C₂H₅ | C₂H₅ | | | |
| I-19 | Cl | Cl | Cl | C₂H₅ | C₂H₅ | | | |
| I-20 | Cl | Cl | CN | C₂H₅ | C₂H₅ | B | 1.119 | 524.9 |
| I-21 | Me | Cl | CF₃ | CH(CH₃)₂ | CH(CH₃)₂ | B | 1.303 | 574.3 |
| I-22 | Me | Cl | CHF₂ | CH(CH₃)₂ | CH(CH₃)₂ | B | 1.225 | 556.3 |
| I-23 | Me | Cl | Br | CH(CH₃)₂ | CH(CH₃)₂ | logP: 2.9 [pH = 10.0] | | |
| I-24 | Me | Cl | Cl | CH(CH₃)₂ | CH(CH₃)₂ | | | |
| I-25 | Me | Cl | CN | CH(CH₃)₂ | CH(CH₃)₂ | B | 1.19 | 531.3 |
| I-26 | Cl | Cl | CF₃ | CH(CH₃)₂ | CH(CH₃)₂ | A | 3.835 | 596.05 |
| I-27 | Cl | Cl | CHF₂ | CH(CH₃)₂ | CH(CH₃)₂ | B | 1.24 | 578 |
| I-28 | Cl | Cl | Br | CH(CH₃)₂ | CH(CH₃)₂ | A | 3.538 | 605.80 |
| I-29 | Cl | Cl | Cl | CH(CH₃)₂ | CH(CH₃)₂ | | | |
| I-30 | Cl | Cl | CN | CH(CH₃)₂ | CH(CH₃)₂ | B | 1.209 | 553.1 |
| I-31 | Br | Br | CF₃ | C₂H₅ | C₂H₅ | B | 1.218 | 655.9 |
| I-32 | Br | Br | CHF₂ | C₂H₅ | C₂H₅ | B | 1.171 | 638.1 |
| I-33 | Br | Br | Br | C₂H₅ | C₂H₅ | | | |
| I-34 | Br | Br | Cl | C₂H₅ | C₂H₅ | | | |
| I-35 | Br | Br | CN | C₂H₅ | C₂H₅ | | | |
| I-36 | Br | Br | CF₃ | CH(CH₃)₂ | CH(CH₃)₂ | A | 3.665 | 683.90 |
| I-37 | Br | Br | CHF₂ | CH(CH₃)₂ | CH(CH₃)₂ | B | 1.245 | 666.1 |
| I-38 | Br | Br | Br | CH(CH₃)₂ | CH(CH₃)₂ | | | |
| I-39 | Br | Br | Cl | CH(CH₃)₂ | CH(CH₃)₂ | | | |
| I-40 | Br | Br | CN | CH(CH₃)₂ | CH(CH₃)₂ | | | |

B. Biological Examples

B.1 Testing for Cross-Resistance to Diamide-Resistant Diamond Back Moth (Plutella Xylostella)

Compounds of formula I, clorantraniliprole, cyantraniliprole and flubendiamide were dissolved using pure analytical acetone and then diluted with 50:50 distilled water: pure analytical acetone. Kinetic HV was added as surfactant at 0.01% v/v.

Cabbage leaves were cut into discs and dipped into serially prepared test solutions (6-8 test concentrations). Treated leaves were air-dried in petri dishes (150×20 mm) lined with moistened filter paper. Each treatment concentration was replicated 3× and after air drying of about an hour, each replicate was inoculated with 10 third instar larvae of (Plutella xylostella). After inoculation, each dish was covered with soft tissue paper with the plate cover on its top and then transferred in a room maintained at 24° C. and 64% relative humidity. Assessment of larvae mortality was recorded 3 days after inoculation (3 DAI).

Collection of Diamond back moth pupae from Cebu region in the Philippines was done in May 2010 and in the last week of March 2012 with F1 and F2 generations used in the assay. The new LC₅₀/LC₉₀ values generated with the Cebu strain are shown in table B.2A and B.2B. The Diamond back moths collected from the Cebu region (Philippines) are reported to show diamide cross resistance, as for example reported by the Insecticide Resistance Action Committee (http://www.irac-online.org/).

B.1A: LC₅₀/LC₉₀ values for compounds of formula I on
Plutella xylostella (Cebu Strain). 3DAI. May 2011

| Compound | LC₅₀ (ppm) | LC₉₀ (ppm) |
|---|---|---|
| I-11 | 2.22 | 4.93 |
| I-26 | 0.82 | 1.55 |
| I-16 | 1.44 | 3.06 |
| Chlorantraniliprole | 16.1 | 37.3 |

| | |
|---|---|
| Flubendiamide | insufficient control at 1000 ppm |

B.1B: LC₅₀/LC₉₀ values for compounds of formula I on
Plutella xylostella (Cebu Strain). 3DAI. May 2012

| Compound | LC₅₀ (ppm) | LC₉₀ (ppm) |
|---|---|---|
| I-21 | 2.64 | 8.37 |
| Chlorantraniliprole | 40.7 | 98.6 |
| Cyantraniliprole | 25.7 | 97.0 |
| Flubendiamide | 902 | 2703 |

As can be seen from tables B.1A & B.1 B, commercial diamide chemistries like flubendiamide show only weak efficacies against the field collected Cebu strain of Plutella xylostella. Compounds of formula I show higher efficacies by at least one order of magnitude compared to flubendiamide, chlorantraniliprole and cyantraniliprole, respectively, when comparing LC₅₀s and LC₉₀s.

LC₅₀ defines the lethal concentration where 50% of a respective population is killed. LC₉₀ defines the lethal concentration where 90% of a respective population is killed.

1-19. (canceled)

20. A method of controlling insects, which are resistant to a ryanodine-modulator insecticide, which method comprises applying to said ryanodine-modulator insecticide resistant insects with at least one pesticidally active anthranilamide compound of formula (I): 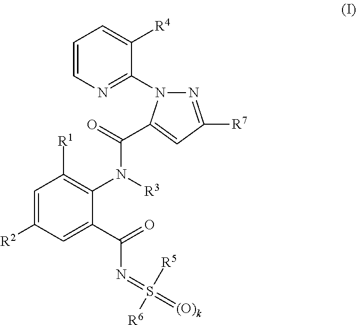
wherein
R¹ is selected from the group consisting of halogen, methyl and halomethyl;
R² is selected from the group consisting of hydrogen, halogen, halomethyl and cyano;
R³ is selected from hydrogen, C₁-C₆ alkyl, C₁-C₆ haloalkyl, C₂-C₆-alkenyl, C₂-C₆-haloalkenyl, C₂-C₆-alkinyl, C₂-C₆-haloalkinyl, C₃-C₈-cycloalkyl, C₃-C₈-halocycloalkyl, C₁-C₄-alkoxy-C₁-C₄-alkyl, C₁-C₄-haloalkoxy-C₁-C₄-alkyl, C(═O)R^{a}, C(═O)OR^{b} and C(═O)NR^{c}R^{d};
R⁴ is hydrogen or halogen;
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
R⁷ is selected from the group consisting of bromo, chloro, difluoromethyl, trifluoromethyl, nitro, cyano, OCH₃, OCHF₂, OCH₂F, OCH₂CF₃, S(═O)ₙCH₃, and S(═O)ₙCF₃;
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a CO═ group, and/or the aliphatic and cycloaliphatic moieties of the aforementioned radicals may be unsubstituted, partially or fully halogenated and/or may carry 1 or 2 substituents selected from C₁-C₄ alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; or
R^{e} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a CO═ group, and/or the aliphatic and cycloaliphatic moieties of the aforementioned radicals may be unsubstituted, partially or fully halogenated and/or may carry 1 or 2 radicals selected from C₁-C₄ alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};
k is 0 or 1;
n is 0, 1 or 2;
or a stereoisomer, salt, tautomer or N-oxide, or a polymorphic crystalline form, a co-crystal or a solvate of a compound or a stereoisomer, salt, tautomer or N-oxide thereof.

21. Method according to claim 20, in which the compound of formula I is a compound of formula IA: 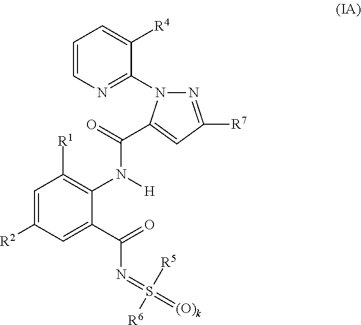
wherein
R⁴ is halogen.

22. Method according to claim 20, in which the compound of formula I is a compound of formula IB: 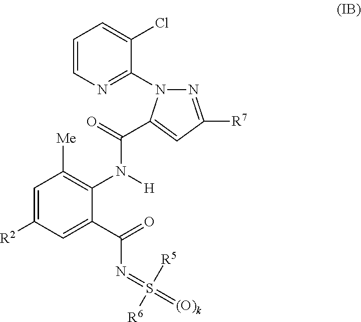
wherein
R² is selected from the group consisting of bromo, chloro, cyano;
R⁷ is selected from the group consisting of bromo, chloro, trifluoromethyl. OCHF₂.

23. Method according to claim 20, in which the compound of formula I is a compound of formula IC: 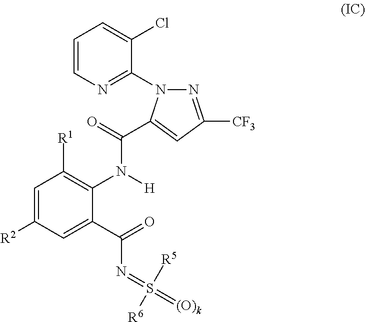
wherein
R¹ is selected from the group consisting of halogen and halomethyl;
R² is selected from the group consisting of bromo, chloro and cyano.

24. Method according to claim 20, in which the compound of formula I is a compound of formula ID: 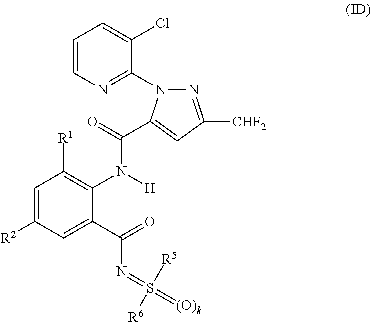
wherein
R¹ is selected from the group consisting of halogen, methyl and halomethyl;
R² is selected from the group consisting of bromo, chloro and cyano.

25. Method according to claim 20, in which in the compound of formula I
R⁵ and R⁶ are selected from the group consisting of methyl, ethyl, isopropyl, n-propyl, n-butyl, isobutyl, tert-butyl, cyclopropyl, and cyclopropylmethyl.

26. Method according to claim 20, in which in the compound of formula I
R⁵ and R⁶ are identical.

27. Method according to claim 20, wherein the ryanodine-modulator insecticide resistant insect is from the order Lepidoptera, Coleoptera or Diptera.

28. Method according to claim 20, wherein the ryanodine-modulator insecticide resistant insect is selected from thrips, hoppers and whitefly.

29. Method of protecting a crop of useful plants susceptible to and/or under attack by insects, which are resistant to a ryanodine-modulator insecticide, which method comprises applying to said crop, treating a plant propagation material of said crop with, and/or applying to said ryanodine-modulator insecticide resistant insects, a compound of formula I as defined in claim 20.

30. Method of controlling resistance to one or more ryanodine-modulator insecticides in insects, which comprises alternately applying a compound of formula I as defined in claim 20, and the ryanodine-modulator insecticide, towards which the insects are resistant, to said insects or to a crop of useful plants susceptible to and/or under attack from said insects.

31. Method according to claim 29, wherein the ryanodine-modulator insecticide resistant insect is from the order Lepidoptera, Coleoptera or Diptera or is selected from thrips, hoppers and whitefly.

32. Method according to claim 29 of protecting a crop of useful plants susceptible to and/or under attack by insects, which insects are resistant to a ryanodine-modulator insecticide, and enhancing the crop, which method comprises applying to the propagation material of said crop an insecticide followed by the foliar application of a compound of the formula (I), as defined in claim 20, beginning with the 3-to 5-leaf crop stage.

33. Method according to claim 20, wherein said insect is one or more of Tuta absoluta, Pieris rapae, Trichoplusia ni, Plutella xylostella, Spodoptera littoralis, Spodoptera frugiperda, Crocidolomia pavonana, Cnaphalocerus medinalis, Sesamia inferens, Chilo suppressalis, Pyrausta furnacalis, Thermesia gemmatalis, Liriomyza sp., Leptinotarsus decemlineata, Epitrix sp., Phyllotreta cruciferae, Meligethes aeneus, Hypera brunneipennis; Nilaparvata lugens, Nephotettix virens;
Acyrthosiphum pisum, Bemisia tabaci, Bemisia argentifolii, Euschisuts heros, Nezara viridula, Piezodurus guildingi, Lygus hesperus, thrips spp., Frankliniella occidentalis; Agriothes sp., Agrotis ypsilon, Phyllotreta spp.;
Cydia pomonella, Elasmopalpus lignosellus, Heliothis armigera, Heliothis virescens, Heliothis zea;
Agriotes lineatus, Agriotes obscurus, Anthonomus grandis, Diabrotica longicornis, Diabrotica semipunctata, Diabrotica 12-punctata, Diabrotica speciosa, Diabrotica virgifera, Epitrix hirtipennis, Phaedon cochlearieae, Phyllotreta striolata.

34. Method according to claim 33, wherein said insect is one or more of Tuta absoluta, Pieris rapae, Trichoplusia ni, Plutella xylostella, Chilo suppressalis, Liriomyza sp., Leptinotarsus decemlineata, Epitrix sp., Phyllotreta cruciferae, Franklinella occidentalis, Bemisia tabaci, Bemisia argentifolii, Agrotis ypsilon.

35. Method according to claim 33, wherein said insect is one or more of Agrotis ypsilon, Heliothis virescens, Plutella xylostella, Agriotes lineatus, Diabrotica virgifera, Hypera brunneipennis, Leptinotarsus decemlineata, Phyllotreta striolata.

36. Method according to claim 33, wherein the insecticide to which the insect is resistant is selected from the group consisting of flubendiamide, chlorantraniliprole (rynaxypyr®), cyantraniliprole (cyazypyr®), phthalamide compounds, M.26.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid,
M.26.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, anthranilamide compounds, M.26.3: 3-bromo-N-{2-bromo-4-chloro-6-[(1-cyclopropylethyl)carbamoyl]phenyl}-1-(3-chlorpyridin-2-yl)-1H-pyrazole-5-carboxamide, and M.26.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate.

37. Method according to claim 36, wherein the ryanodine-modulator insecticide to which the insect is resistant is selected from chlorantraniliprole or cyantraniliprole.

38. Method according to claim 29, wherein the compound of formula I is combined with one or more other pesticidally active compound(s) II selected from insecticides or fungicides.
